SEQUENCE LISTING (45 sequences)

SEQ ID NO: 1 - DNA, 846 bp, Mus musculus
atgaatcagattcaagtcttcatttcaggcctcatactgcttctcccaggcgctgtggattcttatgtggaagtaaagggggtggtgggtcaccctgtcacacttccatgtacttactcaacatatcgtggaatcacaacgacatgttggggccgagggcaatgcccatcttctgcttgtcaaaatacacttatttggaccaatggacatcgtgtcacctatcagaagagcagtcggtacaacttaaaggggcatatttcagaaggagatgtgtccttgacgatagagaactctgttgagagtgacagtggtctgtattgttgtcgagtggagattcctggatggtttaatgatcagaaagtgaccttttcattgcaagttaaaccagagattcccacacgtcctccaagaagacccacaactacaaggcccacagctacaggaagacccacgactatttcaacaagatccacacatgtaccaacatcaaccagagtctctacctccactcctccaacatctacacacacatggactcacaaaccagactggaatggcactgtgacatcctcaggagatacctggagtaatcacactgaagcaatccctccagggaagccgcagaaaaaccctactaagggcttctatgttggcatctgcatcgcagccctgctgctactgctccttgtgagcaccgtggctatcaccaggtacatacttatgaaaaggaagtcagcatctctaagcgtggttgccttccgtgtctctaagattgaagctttgcagaacgcagcggttgtgcattcccgagctgaagacaacatctacattgttgaagatagacct

SEQ ID NO: 2 - DNA, 915 bp, Mus musculus
atgaatcagattcaagtcttcatttcaggcctcatactgcttctcccaggcactgtggattcttatgtggaagtaaagggggtagtgggtcaccctgtcacacttccatgtacttactcaacatatcgtggaatcacaacgacatgttggggccgagggcaatgcccatcttctgcttgtcaaaatacacttatttggaccaatggacatcgtgtcacctatcagaagagcagtcggtacaacttaaaggggcatatttcagaaggagatgtgtccttgacgatagagaactctgttgagagtgacagtggtctgtattgttgtcgagtggagattcctggatggtttaatgatcagaaagtgaccttttcattgcaagttaaaccagagattcccacacgtcctccaacaagacccacaactacaaggcccacagctacaggaagacccacgactatttcaacaagatccacacatgtaccaacatcaatcagagtctctacctccactcctccaacatctacacacacatggactcacaaaccagaacccactacattttgtccccatgagacaacagctgaggtgacaggaatcccatcccatactcctacagactggaatggcactgtgacatcctcaggagatacctggagtaatcacactgaagcaatccctccagggaagccgcagaaaaaccctactaagggcttctatgttggcatctgcatcgcagccctgctgctactgctccttgtgagcaccgtggctatcaccaggtacatacttatgaaaaggaagtcagcatctctaagcgtggttgccttccgtgtctctaagattgaagctttgcagaacgcagcggttgtgcattcccgagctgaagacaacatctacattgttgaagatagacct

SEQ ID NO: 3 - protein, 281 aa, Mus musculus
MNQIQVFISGLILLLPGAVDSYVEVKGVVGHPVTLPCTYSTYRGITTTCWGRGQCPSSACQNTLIWTNGHRVTYQKSSRYNLKGHISEGDVSLTIENSVESDSGLYCCRVEIPGWFNDQVTFSLQVKPEIPTRPPRRPTTTRPTATGRPTTISTRSTHVPTSTRVSTSTPPTSTHTWTHKPDWNGTVTSSGDTWSNHTEAIPPGKPQKNPTKGFYVGICIAALLLLLLVSTVAITRYILMKRKSASLSVVAFRVSKIEALQNAAVVHSRAEDNIYIVEDRP

SEQ ID NO: 4 - protein, 304 aa, Mus musculus
MNQIQVFISGLILLLPGTVDSYVEVKGVVGHPVTLPCTYSTYRGITTTCWGRGQCPSSACQNTLIWTNGHRVTYQKSSRYNLKGHISEGDVSLTIENSVESDSGLYCCRVEIPGWFNDQVTFSLQVKPEIPTRPPTRPTTTRPTATGRPTTISTRSTHVPTSIRVSTSTPPTSTHTWTHKPEPTTFCPHETTAEVTGIPSHTPTDWNGTVTSSGDTWSNHTEAIPPGKPQKNPTKGFYVGICIAALLLLLLVSTVAITRYILMKRKSASLSVVAFRVSKIEALQNAAVVHSRAEDNIYIVEDRP

SEQ ID NO: 5 - protein, 365 aa, Mus musculus
MPMGSLQPLATLYLLGMLVASCLGYVEVKGVVGHPVTLPCTYSTYRGITTTCWGRGQCPSSACQNTLIWTNGHRVTYQKSSRYNLKGHISEGDVSLTIENSVESDSGLYCCRVEIPGWFNDQKVTFSLQVKPDPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 6 - DNA, 920 bp, Mus musculus
  misc_feature (1)...(920): n = A,T,C or G
taagttgaacatatacgatcaggcaaaaggatggtagaactagagagactcagttttcaaacaaaatattgaaggtgtggccaggagataaggatggaattcntgggaccaagattcctttttatctatcagcagnctccatcagcagcatgaatcagattcaagtcttcatttcaggcctcatactgcttctcccaggtgccgtggagtctcatacagcagtgcaggggctggcgggtcaccctgtcacacttccatgtatttattcgacacaccttggtggaatcgttcctatgtgttggggcctaggggaatgccgccattcttattgtatacggtcacttatctggaccaatggatatacggtcacacatcagaggaacagtcgataccagctaaaggggaatatttcagaaggaaatgtgtccttgaccatagagaacactgttgtgggtgatggtggtccctattgctgtgtagtggagatacctggagcgttccattttgtggactatatgttggaagttaaaccagaaatttccacgagtccaccaacaaggcccacagctacaggaagacccacaactatttcaacaagatccacacatgtaccaacatcaaccagagtctctacctctacttctccaacaccagcacacacagagacctacaaaccagaggccactacattttatccagatcagactacagctgaggtgacagaaaccttaccctctactcctgcagactggcataacactgtgacatcctcagatgacccttgggatgataacactgaagtaatccctccacagaagccacagaaaaacctgaataagggcttctatgttggcatctccattgcagccctgctgatattgatgcttctgagcaccatggttatcaccaggtacgtggtt

SEQ ID NO: 7 - protein, 305 aa, Mus musculus
  VARIANT (1)...(305): TIM-1 BALB/c allele
MNQIQVFISGLILLLPGTVDSYVEVKGVVGHPVTLPCTYSTYRGITTTCWGRGQCPSSACQNTLIWTNGHRVTYQKSSRYNLKGHISEGDVSLTIENSVESDSGLYCCRVEIPGWFNDQKVTFSLQVKPEIPTRPPTRPTTTRPTATGRPTTISTRSTHVPTSIRVSTSTPPTSTHTWTHKPEPTTFCPHETTAEVTGIPSHTPTDWNGTVTSSGDTWSNHTEAIPPGKPQKNPTKGFYVGICIAALLLLLLVSTVAITRYILMKRKSASLSVVAFRVSKIEALQNAAVVHSRAEDNIYIVEDRP

SEQ ID NO: 8 - DNA, 918 bp, Mus musculus
atgaatcagattcaagtcttcatttcaggcctcatactgcttctcccaggcactgtggattcttatgtggaagtaaagggggtagtgggtcaccctgtcacacttccatgtacttactcaacatatcgtggaatcacaacgacatgttggggccgagggcaatgcccatcttctgcttgtcaaaatacacttatttggaccaatggacatcgtgtcacctatcagaagagcagtcggtacaacttaaaggggcatatttcagaaggagatgtgtccttgacgatagagaactctgttgagagtgacagtggtctgtattgttgtcgagtggagattcctggatggtttaatgatcagaaagtgaccttttcattgcaagttaaaccagagattcccacacgtcctccaacaagacccacaactacaaggcccacagctacaggaagacccacgactatttcaacaagatccacacatgtaccaacatcaatcagagtctctacctccactcctccaacatctacacacacatggactcacaaaccagaacccactacattttgtccccatgagacaacagctgaggtgacaggaatcccatcccatactcctacagactggaatggcactgtgacatcctcaggagatacctggagtaatcacactgaagcaatccctccagggaagccgcagaaaaaccctactaagggcttctatgttggcatctgcatcgcagccctgctgctactgctccttgtgagcaccgtggctatcaccaggtacatacttatgaaaaggaagtcagcatctctaagcgtggttgccttccgtgtctctaagattgaagctttgcagaacgcagcggttgtgcattcccgagctgaagacaacatctacattgttgaagatcgaccttga

SEQ ID NO: 9 - protein, 282 aa, Mus musculus
  VARIANT (1)...(282): TIM-1,C.D2 ES-HBA AND DBA/2J allele
MNQIQVFISGLILLLPGAVDSYVEVKGVVGHPVTLPCTYSTYRGITTTCWGRGQCPSSACQNTLIWTNGHRVTYQKSSRYNLKGHISEGDVSLTIENSVESDSGLYCCRVEIPGWFNDQKVTFSLQVKPEIPTRPPRRPTTTRPTATGRPTTISTRSTHVPTSTRVSTSTPPTSTHTWTHKPDWNGTVTSSGDTWSNHTEAIPPGKPQKNPTKGFYVGICIAALLLLLLVSTVAITRYILMKRKSASLSVVAFRVSKIEALQNAAVVHSRAEDNIYIVEDRP

SEQ ID NO: 10 - DNA, 849 bp, Mus musculus
atgaatcagattcaagtcttcatttcaggcctcatactgcttctcccaggcgctgtggattcttatgtggaagtaaagggggtggtgggtcaccctgtcacacttccatgtacttactcaacatatcgtggaatcacaacgacatgttggggccgagggcaatgcccatcttctgcttgtcaaaatacacttatttggaccaatggacatcgtgtcacctatcagaagagcagtcggtacaacttaaaggggcatatttcagaaggagatgtgtccttgacgatagagaactctgttgagagtgacagtggtctgtattgttgtcgagtcgagattcctggatggtttaatgatcagaaagtgaccttttcattgcaagttaaaccagagattcccacacgtcctccaagaagacccacaactacaaggcccacagctacaggaagacccacgactatttcaacaagatccacacatgtaccaacatcaaccagagtctctacctccactcctccaacatctacacacacatggactcacaaaccagactggaatggcactgtgacatcctcaggagatacctggagtaatcacactgaagcaatccctccagggaagccgcagaaaaaccctactaagggcttctatgttggcatctgcatcgcagccctgctgctactgctccttgtgagcaccgtggctatcaccaggtacatacttatgaaaaggaagtcagcatctctaagcgtggttgccttccgtgtctctaagattgaagctttgcagaacgcagcggttgtgcattcccgagctgaagacaacatctacattgttgaagatagaccttga

SEQ ID NO: 11 - protein, 305 aa, Mus musculus
  VARIANT (1)...(305): TIM-2 BALB/c allele
MNQIQVFISGLILLLPGAVESHTAVQGLAGHPVTLPCIYSTHLGGIVPMCWGLGECRHSYCIRSLIWTNGYTVTHQRNSRYQLKGNISEGNVSLTIENTVVGDGGPYCCVVEIPGAFHFVDYMLEVKPEISTSPPTRPTATGRPTTISTRSTHVPTSTRVSTSTSPTPAHTETYKPEATTFYPDQTTAEVTETLPSTPADWHNTVTSSDDPWDDNTEVIPPQKPQKNLNKGFYVGISIAALLILMLLSTMVITRYVVMKRKSESLSFVAFPISKIGASPKKVVERTRCEDQVYIIEDTPYPEEES

SEQ ID NO: 12 - DNA, 958 bp, Mus musculus
aagctacggctctctcctaactggtcgtaccatgaatcagattcaagtcttcatttcaggcctcatactgcttctcccaggtgccgtggagtctcatacagcagtgcaggggctggcgggtcaccctgtcacacttccatgtatttattcgacacaccttggtggaatcgttcctatgtgttggggcctaggggaatgccgccattcttattgtatacggtcacttatctggaccaatggatatacggtcacacatcagaggaacagtcgataccagctaaaggggaatatttcagaaggaaatgtgtccttgaccatagagaacactgttgtgggtgatggtggtccctattgctgtgtagtggagatacctggagcgttccattttgtggactatatgttggaagttaaaccagaaatttccacgagtccaccaacaaggcccacagctacaggaagacccacaactatttcaacaagatccacacatgtaccaacatcaaccagagtctctacctctacttctccaacaccagcacacacagagacctacaaaccagaggccactacattttatccagatcagactacagctgaggtgacagaaaccttaccctctactcctgcagactggcataacactgtgacatcctcagatgacccttgggatgataacactgaagtaatccctccacagaagccacagaaaaacctgaataagggcttctatgttggcatctccattgcagccctgctgatattgatgcttctgagcaccatggttatcaccaggtacgtggttatgaaaaggaagtcagaatctctgagctttgttgccttccctatctctaagattggagcttcccccaaaaaagtggtcgaacggaccagatgtgaagaccaggtctacattattgaagacactccttaccctgaagaagagtcctagtgcctctac

SEQ ID NO: 13 - protein, 305 aa, Mus musculus; identical to SEQ ID NO: 11
  VARIANT (1)...(305): TIM-2, C.D2 ES-HBA AND DBA/2J allele

SEQ ID NO: 14 - DNA, 958 bp, Mus musculus
aagctacggctctctcctaactggtcgtaccatgaatcagattcaagtcttcatttcaggcctcatactgcttctcccaggtgccgtggagtctcatacagcagtgcaggggctggcgggtcaccctgtcacacttccatgtatttattcgacacaccttggtggaatcgttcctatgtgttggggcctaggggaatgccgccattcttattgtatacggtcacttatctggaccaatggatatacggtcacacatcagaggaacagtcgataccagctaaaggggaatatttcagaaggaaatgtgtccttgaccatagagaacactgttgtgggtgatggtggtccctattgctgtgtagtggagatacctggagcgttccattttgtggactatatgttggaagttaaaccagaaatttccacgagtccaccaacaaggcccacagctacaggaagacccacaactatttcaacaagatccacacatgtaccaacatcaaccagagtctctacctctacttctccaacaccagcacacacagagacctacaaaccagaggccactacattttatccagatcagactacagctgaggtgacagaaaccttaccctctactcctgcagactggcataacactgtgacatcctcagatgacccttgggatgataacactgaagtaatccctccacagaagccacagaaaaacctgaataagggcttctatgttggcatctccattgcagccctgctgatattgatgcttctgagcaccatggttatcaccaggtacgtggttatgaaaaggaagtcagaatctctgagcttcgttgccttccctatctctaagattggagcttcccccaaaaaagtggtcgaacggaccagatgtgaagaccaggtctacattattgaagacactccttaccccgaagaagagtcctagtgcctctac

SEQ ID NO: 15 - protein, 281 aa, Mus musculus
  VARIANT (1)...(281): TIM-3 BALB/c allele
MFSGLTLNCVLLLLQLLLARSLEDGYKVEVGKNAYLPCSYTLPTSGTLVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKDSGETIRTAIHIGVGVSAGLTLALIIGVLILKWYSCKKKKLSSLSLITLANLPPGGLANAGAVRIRSEENIYTIEENVYEVENSNEYYCYVNSQQPS

SEQ ID NO: 16 - DNA, 2725 bp, Mus musculus
ttttaaccgaggagctaaagctatccctacacagagctgtccttggatttcccctgccaagtactcatgttttcaggtcttaccctcaactgtgtcctgctgctgctgcaactactacttgcaaggtcattggaagatggttataaggttgaggttggtaaaaatgcctatctgccctgcagttacactctacctacatctgggacacttgtgcctatgtgctggggcaagggattctgtccttggtcacagtgtaccaatgagttgctcagaactgatgaaagaaatgtgacatatcagaaatccagcagataccagctaaagggcgatctcaacaaaggagatgtgtctctgatcataaagaatgtgactctggatgaccatgggacctactgctgcaggatacagttccctggtcttatgaatgataaaaaattagaactgaaattagacatcaaagcagccaaggtcactccagctcagactgcccatggggactctactacagcttctccaagaaccctaaccacggagagaaatggttcagagacacagacactggtgaccctccataataacaatggaacaaaaatttccacatgggctgatgaaattaaggactctggagaaacgatcagaactgctatccacattggagtgggagtctctgctgggttgaccctggcacttatcattggtgtcttaatccttaaatggtattcctgtaagaaaaagaagttatcgagtttgagccttattacactggccaacttgcctccaggagggttggcaaatgcaggagcagtcaggattcgctctgaggaaaatatctacaccatcgaggagaacgtatatgaagtggagaattcaaatgagtactactgctacgtcaacagccagcagccatcctgaccgcctctggactgccacttttaaaggctcgccttcatttctgactttggtatttccctttttgaaaactatgtgatctgtcacttggcaacctcattggaggttctgaccacagccactgagaaaagagttccagttttctggggataattaactcacaaggggattcgactgtaactcatgctacattgaaatgctccattttatccctgagtttcagggatcggatctcccactccagagacttcaatcatgcgtgttgaagctcactcgtgctttcatacattaggaatggttagtgtgatgtctttgagacatagaggtttgtggtatatccgcaaagctcctgaacaggtagggggaataaagggctaagataggaaggtgcggttctttgttgatgttgaaaatctaaagaagttggtagcttttctagagatttctgaccttgaaagattaagaaaaagccaggtggcatatgcttaacacgatataacttgggaaccttaggcaggagggtgataagttcaaggtcagccagggctatgctggtaagactgtctcaaaatccaaagacgaaaataaacatagagacagcaggaggctggagatgaggctcggacagtgaggtgcattttgtacaagcacgaggaatctatatttgatcgtagaccccacatgaaaaagctaggcctggtagagcatgcttgtagactcaagagatggagaggtaaaggcacaacagatccccggggcttgcgtgcagtcagcttagcctaggtgctgagttccaagtccacaagagtccctgtctcaaagtaagatggactgagtatctggcgaatgtccatgggggttgtcctctgctctcagaagagacatgcacatgaacctgcacacacacacacacacacacacacacacacacacacacacacacacatgaaatgaaggttctctctgtgcctgctacctctctataacatgtatctctacaggactctcctctgcctctgttaagacatgagtgggagcatggcagagcagtccagtaattaattccagcactcagaaggctggagcagaagcgtggagagttcaggagcactgtgcccaacactgccagactcttcttacacaagaaaaaggttacccgcaagcagcctgctgtctgtaaaaggaaaccctgcgaaaggcaaactttgactgttgtgtgctcaaggggaactgactcagacaacttctccattcctggaggaaactggagctgtttctgacagaagaacaaccggtgactgggacatacgaaggcagagctcttgcagcaatctatatagtcagcaaaatattctttgggaggacagtcgtcaccaaattgatttccaagccggtggacctcagtttcatctggcttacagctgcctgcccagtgcccttgatctgtgctggctcccatctataacagaatcaaattaaatagaccccgagtgaaaatattaagtgagcagaaaggtagctttgttcaaagatttttttgcattggggagcaactgtgtacatcagaggacatctgttagtgaggacaccaaaacctgtggtaccgttttttcatgtatgaattttgttgtttaggttgcttctagctagctgtggaggtcctggctttcttaggtgggtatggaagggagaccatctaacaaaatccattagagataacagctctcatgcagaagggaaaactaatctcaaatgttttaaagtaataaaactgtactggcaaagtactttgagcatatttaaaaaaaaaaaaaaa

SEQ ID NO: 17 - protein, 281 aa, Mus musculus
  VARIANT (1)...(281): TIM-3, C.D2 ES-HBA AND DBA/2J allele
MFSGLTLNCVLLLLQLLLARSLENAYVFEVGKNAYLPCSYTLSTPGALVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKDSGETIRTAIHIGVGVSAGLTLALIIGVLILKWYSCKKKKLSSLSLITLANLPPGGLANAGAVRIRSEENIYTIEENVYEVENSNEYYCYVNSQQPS

SEQ ID NO: 18 - DNA, 862 bp, Mus musculus
cccctcccaagtactcatgttttcaggtcttaccctcaactgtgtcctgctgctgctgcaactactacttgcaaggtcattggaaaatgcttatgtgtttgaggttggtaagaatgcctatctgccctgcagttacactctatctacacctggggcacttgtgcctatgtgctggggcaagggattctgtccttggtcacagtgtaccaacgagttgctcagaactgatgaaagaaatgtgacatatcagaaatccagcagataccagctaaagggcgatctcaacaaaggagacgtgtctctgatcataaagaatgtgactctggatgaccatgggacctactgctgcaggatacagttccctggtcttatgaatgataaaaaattagaactgaaattagacatcaaagcagccaaggtcactccagctcagactgcccatggggactctactacagcttctccaagaaccctaaccacggagagaaatggttcagagacacagacactggtgaccctccataataacaatggaacaaaaatttccacatgggctgatgaaattaaggactctggagaaacgatcagaactgctatccacattggagtgggagtctctgctgggttgaccctggcacttatcattggtgtcttaatccttaaatggtattcctgtaagaaaaagaagttatcgagtttgagccttattacactggccaacttgcctccaggagggttggcaaatgcaggagcagtcaggattcgctctgaggaaaatatctacaccatcgaggagaacgtatatgaagtggagaattcaaatgagtactactgctacgtcaacagccagcagccatcctga

SEQ ID NO: 19 - protein, 345 aa, Mus musculus
  VARIANT (1)...(345): TIM-4, BALB/c allele
MSKGLLLLWLVTELWWLYLSKSPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAITTESETLPASNHSQRSMMTISTDIAVLRPTGSNPGILPSTSQLTTQKTTLTTSESLQKTTKSHQINSRQTILIIACCVGFVLMVLLFLAFLLRGKVTGANCLQRHKRPDNTEVSDSFLNDISHGRDDEDGIFTL

SEQ ID NO: 20 - DNA, 1032 bp, Mus musculus
atgtccaaggggcttctcctcctctggctggtgacggagctctggtggctttatctgacaccagctgcctcagaggatacaataatagggtttttgggccagccggtgactttgccttgtcattacctctcgtggtcccagagccgcaacagtatgtgctggggcaaaggttcatgtcccaattccaagtgcaatgcagagcttctccgtacagatggaacaagaatcatctccaggaagtcaacaaaatatacacttttggggaaggtccagtttggtgaagtgtccttgaccatctcaaacaccaatcgaggtgacagtggggtgtactgctgccgtatagaggtgcctggctggttcaatgatgtcaagaagaatgtgcgcttggagctgaggagagccacaacaaccaaaaaaccaacaacaaccacccggccaaccaccaccccttatgtaaccaccaccaccccagagctgcttccaacaacagtcatgaccacatctgttcttccaaccaccacaccaccccagacactagccaccactgccttcagtacagcagtgaccacgtgcccctcaacaacacctggctccttctcacaagaaaccacaaaagggtccgccatcactacagaatcagaaactctgcctgcatccaatcactctcaaagaagcatgatgaccatatctacagacatagccgtactcaggcccacaggctctaaccctgggattctcccatccacttcacagctgacgacacagaaaacaacattaacaacaagtgagtctttgcagaagacaactaaatcacatcagatcaacagcagacagaccatcttgatcattgcctgctgtgtgggatttgtgctaatggtgttattgtttctggcgtttctccttcgagggaaagtcacaggagccaactgtttgcagagacacaagaggccagacaacactgaagatagtgacagcgtcctcaatgacatgtcacacgggagggatgatgaagacgggatcttcactctctga

SEQ ID NO: 21 - protein, 345 aa, Mus musculus
  VARIANT (1)...(345): C.D2 ES-HBA and DBA/2J allele
MSKGLLLLWLVMELWWLYLSKSPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLRGKVTGANCLQRHKRPDNTEVSDSFLNDISHGRDDEDGIFTL

SEQ ID NO: 22 - DNA, 1032 bp, Mus musculus
atgtccaaggggcttctcctcctctggctggtgatggagctctggtggctttatctgacaccagctgcctcagaggatacaataatagggtttttgggccagccggtgactttgccttgtcattacctctcgtggtcccagagccgcaacagtatgtgctggggcaaaggttcatgtcccaattccaagtgcaatgcagagcttctccgtacagatggaacaagaatcatctccaggaagtcaacaaaatatacacttttggggaaggtccagtttggtgaagtgtccttgaccatctcaaacaccaatcgaggtgacagtggggtgtactgctgccgtatagaggtgcctggctggttcaatgatgtcaagaagaatgtgcgcttggagctgaggagagccacaacaaccaaaaaaccaacaacaaccacccggccaaccaccaccccttatgtaaccaccaccaccccagagctgcttccaacaacagtcatgaccacatctgttcttccaaccaccacaccaccccagacactagccaccactgccttcagtacagcagtgaccacgtgcccctcaacaacacctggctccttctcacaagaaaccacaaaagggtccgccttcactacagaatcagaaactctgcctgcatccaatcactctcaaagaagcatgatgaccatatctacagacatagccgtactcaggcccacaggctctaaccctgggattctcccatccacttcacagctgacgacacagaaaacaacattaacaacaagtgagtctttgcagaagacaactaaatcacatcagatcaacagcagacagaccatcttgatcattgcctgctgtgtgggatttgtgctaatggtgttattgtttctggcgtttctccttcgagggaaagtcacaggagccaactgtttgcagagacacaagaggccagacaacactgaagatagtgacagcgtcctcaatgacatgtcacacgggagggatgatgaagacgggatcttcactctctga

SEQ ID NO: 23 - protein, 359 aa, Homo sapiens
  VARIANT (1)...(359): TIM-1 allele 1
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVPTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 24 - DNA, 1080 bp, Homo sapiens
atgcatcctcaagtggtcatcttaagcctcatcctacatctggcagattctgtagctggttctgtaaaggttggtggagaggcaggtccatctgtcacactaccctgccactacagtggagctgtcacatcaatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaacgacaactgttccaacaacaatgagcattccaacgacaacgactgttccgacgacaatgactgtttcaacgacaacgagcgttccaacgacaacgagcattccaacaacaacaagtgttccagtgacaacaacggtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaa

SEQ ID NO: 25 - protein, 359 aa, Homo sapiens
  VARIANT (1)...(359): TIM-1, allele 2
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVPTTMTVSTTTSVPTTTSIPTTTSVPVTTAVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 26 - DNA, 1080 bp, Homo sapiens
atgcatcctcaagtggtcatcttaagcctcatcctacatctggcagattctgtagctggttctgtaaaggttggtggagaggcaggtccatctgtcacactaccctgccactacagtggagctgtcacatcaatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaacgacaactgttccaacaacaatgagcattccaacgacaacgactgttccgacgacaatgactgtttcaacgacaacgagcgttccaacgacaacgagcattccaacaacaacaagtgttccagtgacaacagcggtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaa

SEQ ID NO: 27 - protein, 365 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTTVPTTTVPTTMSIPTTTTVPTTMTVSTTTSVPTTTSIPTTTSVPVTTAVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 28 - DNA, 1098 bp, Homo sapiens
atgcatcctcaagtggtcatcttaagcctcatcctacatctggcagattctgtagctggttctgtaaaggttggtggagaggcaggtccatctgtcacactaccctgccactacagtggagctgtcacatcaatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacaacgactgttccaacgacaactgttccaacaacaatgagcattccaacgacaacgactgttccgacgacaatgactgtttcaacgacaacgagcgttccaacgacaacgagcattccaacaacaacaagtgttccagtgacaacarcggtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaa

SEQ ID NO: 29 - protein, 359 aa, Homo sapiens
  VARIANT (1)...(359): TIM-1, allele 4
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVPTTMTVSTTTSVPTTTSIPTTTSVPVTTSVSTFVPPMPLPRQNHEPVATSPSSPQPAETHPTTLQGTIRREPTSSPLYSYTTDGNDTVTESSDGLWSNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 30 - DNA, 1079 bp, Homo sapiens
atgcatcctcaagtggtcatcttaagcctcatcctacatctggcagattctgtagctggttctgtaaaggttggtggagaggcaggtccatctgtcacactaccctgccactacagtggagctgtcacatcaatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaacgacaactgttccaacaacaatgagcattccaacgacaacggactgttccgacgacaatgactgtttcaacgacaacgagcgttccaacgacaacgagcattccaacaacaacaagtgttccagtgacaacatgtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggartaacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaa

SEQ ID NO: 31 - protein, 364 aa, Homo sapiens [sequence omitted]

SEQ ID NO: 32 - DNA, 1095 bp, Homo sapiens
atgcatcctcaagtggtcatcttaagcctcatcctacatctggcagattctgtagctggttctgtaaaggttggtggagaggcaggtccatctgtcacactaccctgccactacagtggagctgtcacatcaatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacaacgactgttccaacgacaactgttccaacaacaatgagcattccaacgacaacgactgttccgacgacaatgactgtttcaacgacaacgagcgttccaacgacaacgagcattccaacaacaagtgttccagtgacaacaacggtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaa

SEQ ID NO: 33 - protein, 364 aa, Homo sapiens
  VARIANT (1)...(364): TIM-1,allele 6
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLGIVPPKVTTTPIVTTVPTVTTVRTSTTVPTTTTVPMTTTVPTTTVPTTMSIPTTTTVPTTMTVSTTTSVPTTTSIPTTSVPVTTTVSTFVPPMPLPRQNHEPVATSPSSPQPAETHPTTLQGAIRREPTSSPLYSYTTDGDDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 34 - DNA, 1099 bp, Homo sapiens
atgcatcctcaagtggtcatcttaagcctcatcctacatctggcagattctgtagctggttctgtaaaggttggtggagaggcaggtccatctgtcacactaccctgccactacagtggagctgtcacatcaatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacaaccgactgttccaacgacaactgttccaacaacaatgagcattccaacgacaacgactgttccgacgacaatgactgtttcaacgacaacgagcgttccaacgacaacgagcattccaacaacaacaagtgttccagtgacaacaacggtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatggggatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaa

SEQ ID NO: 35 - protein, 301 aa, Homo sapiens
  VARIANT (1)...(301): TIM-3, allele 1
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 36 - DNA, 1116 bp, Homo sapiens
ggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatagatccaaccaccttatttttgagcttggtgttttgtctttttcagaaactatgagctgtgtcacctgactggttttggaggttctgtccactgctatggagcagagttttcccattttcagaagataatgactcacatgggaattgaactggga

SEQ ID NO: 37 - protein, 301 aa, Homo sapiens
  VARIANT (1)...(301): TIM-3, allele
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 38 - DNA, 1116 bp, Homo sapiens
ggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatagatccaaccaccttatttttgagcttggtgttttgtctttttcagaaactatgagctgtgtcacctgactggttttggaggttctgtccactgctatggagcagagttttcccattttcagaagataatgactcacatgggaattgaactggga

SEQ ID NO: 39 - protein, 378 aa, Homo sapiens
  VARIANT (1)...(378): TIM-4, ALLELE 1
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSAESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 40 - DNA, 1156 bp, Homo sapiens
atgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgctgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttag

SEQ ID NO: 41 - protein, 378 aa, Homo sapiens
  VARIANT (1)...(378): TIM-4, allele 2
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 42 - DNA, 1156 bp, Homo sapiens
atgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgttgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttag

SEQ ID NO: 43 - DNA, 64 bp, Artificial Sequence
  synthetic forward primer
tggcaccggtgccaccatgcccatggggtctctgcaaccgctggccaccttgtacctgctgggg

SEQ ID NO: 44 - DNA, 60 bp, Artificial Sequence
  synthetic reverse  primer
taggagatctcctaggcaggaagcgaccagcatccccagcaggtacaaggtggccagcgg

SEQ ID NO: 45 - DNA, 20 bp, Artificial Sequence
  synthetic CpG oligonucleotide
tccatgacgttcctgacgtt

(19) United States
(12) Patent Application Publication
(10) Pub. No.: US 2005/0276756 A1
(43) Pub. Date: Dec. 15, 2005

(54) Compositions as adjuvants to improve immune responses to vaccines and methods of use

(76) Inventors: William Soo Hoo, Carlsbad, CA (US); Eric R. Jensen, San Diego, CA (US); Thomas Moll, San Diego, CA (US); Dennis J. Carlo, Rancho Santa Fe, CA (US); Brian K. Helmich, La Jolla, CA (US); Soonpin Yei, Carlsbad, CA (US); Jayant Thatte, San Diego, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/087,177

(22) Filed: Mar. 22, 2005

Related U.S. Application Data
(60) Provisional application No. 60/555,827, filed on Mar. 24, 2004.
(60) Provisional application No. 60/582,479, filed on Jun. 23, 2004.

(51) Int. Cl. A61K 51/00; A61K 39/395; A61K 35/78; A61K 33/24; A61K 31/704; A61K 31/7048; A61K 31/7076

(52) U.S. Cl. 424/1.49; 424/143.1; 424/236.1; 514/34; 514/50; 514/27; 514/39; 424/649; 424/731; 514/251; 514/410

(57) ABSTRACT

The invention provides compositions containing an antigen and a TIM targeting molecule. The invention additionally provides a TIM targeting molecule conjugate, for example, a TIM targeting molecule targeted to a therapeutic or diagnostic moiety. The invention additionally provides methods of using such compositions. In one embodiment, the invention provides a method of stimulating an immune response in an individual by administering a composition comprising an antigen and a TIM targeting molecule in a pharmaceutically acceptable carrier. In another embodiment, the invention provides a method of stimulating an immune response in an individual by administering an antigen and a TIM targeting molecule, which can be administered together in a single composition or separately.

This application claims the benefit of priority of U.S. Provisional application Ser. No. 60/555,827, filed Mar. 24, 2004, and of U.S. Provisional application Ser. No. 60/582,479, filed Jun. 23, 2004, each of which the entire contents is incorporated herein by reference.

### BACKGROUND OF THE INVENTION

The body's defense against microbes is mediated by early reactions of the innate immune system and by later responses of the adaptive immune system. Innate immunity involves mechanisms that recognize structures which are, for example, characteristic of microbial pathogens and that are not present on mammalian cells. Examples of such structures include bacterial lipopolysaccharides (LPS), viral double stranded RNA and unmethylated CpG DNA nucleotides. The effector cells of the innate immune response comprise neutrophils, macrophages and natural killer cells (NK cells). In addition to innate immunity, vertebrates, including mammals, have evolved immunological defense mechanisms that are stimulated by exposure to infectious agents and that increase in magnitude and effectiveness with each successive exposure to a particular antigen. Due to its capacity to adapt to a specific infection or antigenic insult, this immune defense mechanism has been described as adaptive immunity. There are two types of adaptive immune responses, called humoral immunity, involving antibodies produced by B lymphocytes, and cell-mediated immunity, mediated by T lymphocytes.

Two major types of T lymphocytes have been described: CD8+ cytotoxic T lymphocytes (CTLs) and CD4+ T helper cells (Th cells). CD8+ T cells are effector cells that, via the T cell receptor (TCR), recognize foreign antigens presented by class I MHC molecules on, for instance, virally or bacterially infected cells. Upon recognition of foreign antigens, CD8+ T cells undergo an activation, maturation and proliferation process. This differentiation process results in CTL clones which have the capacity of destroying the target cells displaying foreign antigens. T helper cells on the other hand are involved in both humoral and cell-mediated forms of effector immune responses. With respect to the humoral, or antibody, immune response, antibodies are produced by B lymphocytes through interactions with Th cells. Specifically, extracellular antigens, such as circulating microbes, are taken up by specialized antigen-presenting cells (APCs), processed, and presented in association with class II major histocompatibility complex (MHC) molecules to CD4+ Th cells. These Th cells in turn activate B lymphocytes, resulting in antibody production. The cell-mediated, or cellular, immune response, in contrast, functions to neutralize microbes which inhabit intracellular locations, such as after successful infection of a target cell. Foreign antigens, such as, for example, microbial antigens, are synthesized within infected cells and presented on the surfaces of such cells in association with class I MHC molecules. Presentation of such epitopes leads to the above described stimulation of CD8+ CTLs, a process which in turn is also stimulated by CD4+ Th cells. Th cells are composed of at least two distinct subpopulations, termed Th1 and Th2 cells. The Th1 and Th2 subtypes represent polarized populations of Th cells which differentiate from common precursors after exposure to antigen.

Each T helper cell subtype secretes cytokines that promote distinct immunological effects that are opposed to one another and that cross-regulate each other's expansion and function. Th1 cells secrete high amounts of cytokines such as interferon-gamma (IFN-γ), tumor necrosis factor-alpha (TNF-α), interleukin-2 (IL-2) and IL-12, and low amounts of IL-4. Th1-associated cytokines promote CD8+ cytotoxic T lymphocyte (CTL) activity and are most frequently associated with cell-mediated immune responses against intracellular pathogens. In contrast, Th2 cells secrete high amounts of cytokines such as IL-4, IL-13 and IL-10, but low IFN-γ, and promote antibody responses. Th2 responses are particularly relevant for humoral responses, such as protection from anthrax and for the elimination of helminthic infections.

Whether a resulting immune response is Th1- or Th2-driven largely depends on the pathogen involved and on factors in the cellular environment, such as cytokines. Failure to activate a T helper response, or the correct T helper subset, can result not only in the inability to mount a sufficient response to combat a particular pathogen, but also in the generation of poor immunity against re-infection. Many infectious agents are intracellular pathogens in which cell-mediated responses, as exemplified by Th1 immunity, would be expected to play an important role in protection and/or therapy. Moreover, for many of these infections it was demonstrated that the induction of inappropriate Th2 responses negatively affects disease outcome. Examples include M. tuberculosis, S. mansoni, and also leishmania. Non-healing forms of human and murine leishmaniasis result from strong but counterproductive Th2-like-dominated immune responses. Lepromatous leprosy also appears to feature a prevalent, but inappropriate, Th2-like response. HIV infection represents another example. Here, it has been suggested that a drop in the ratio of Th1-like cells to other Th cell subpopulations can play a critical role in the progression toward disease symptoms.

As a protective measure against infectious agents, vaccination protocols for microbes have been developed. Vaccination protocols against infectious pathogens are often hampered by poor vaccine immunogenicity, an inappropriate type of response (antibody versus cell-mediated immunity), a lack of ability to elicit long-term immunological memory, and/or failure to generate immunity against different serotypes of a given pathogen. Current vaccination strategies target the elicitation of antibodies specific for a given serotype and for many common pathogens, for example, viral serotypes or pathogens. Efforts must be made on a recurring basis to monitor which serotypes are prevalent around the world. An example of this is the annual monitoring of emerging influenza A serotypes that are anticipated to be the major infectious strains.

To support vaccination protocols, adjuvants that would support the generation of immune responses against specific infectious diseases have been developed. For example, aluminum salts have been used as relatively safe and effective vaccine adjuvants to enhance antibody responses to certain pathogens. One of the disadvantages of such adjuvants is that they are relatively ineffective at stimulating a cell-mediated immune response and produce an immune response that is largely Th2 biased.

To increase the effectiveness of an adaptive immune response, such as in a vaccination protocol or during a microbial infection, it is therefore important to develop novel, more effective, vaccine adjuvants. The present invention satisfies this need and provides related advantages as well.

### SUMMARY OF THE INVENTION

The invention provides compositions containing an antigen and a TIM targeting molecule or agent. The invention additionally provides methods of using such compositions. In one embodiment, the invention provides a method of stimulating an immune response in an individual by administering a composition comprising an antigen and a TIM targeting molecule in a pharmaceutically acceptable carrier. In another embodiment, the invention provides a method of stimulating an immune response in an individual by administering an antigen and a TIM targeting molecule, which can be administered together in a single composition or separately.

### BRIEF DESCRIPTION OF THE DRAWINGS

FIG. 1 shows an 846 bp cDNA nucleotide sequence (SEQ ID NO:1) of the mouse C57BL/6 TIM-1 allele. The signal sequence is underlined, the sequences encoding for the mucin domain are italicized, the transmembrane domain is underlined and italicized.

FIG. 2 shows a 915 bp cDNA nucleotide sequence (SEQ ID NO2:) of the mouse BALB/c TIM-1 allele. The signal sequence is underlined, the sequences encoding for the mucin domain are italicized, the transmembrane domain is underlined and italicized.

FIG. 3 shows a protein sequence comparison of the mouse C57B1/6 (B6)(SEQ ID NO:3) and BALB/c (BALB) (SEQ ID NO:4) TIM-1 alleles using the single letter amino acid code. Single amino acid substitutions are marked by a triangle, potential N-glycosylation sites are marked by a star.

FIG. 4 shows an example of a TIM-1/Fc fusion protein, a 365 amino acid protein designated mouse TIM-1 Ig Fc.nl protein (SEQ ID NO:5). The example given is for a precursor polypeptide with a human CD5 leader (underlined), followed by the Ig domain of TIM-1 (plain text) and the Fc region of a point-mutated non-lytic mouse IgG2a Fc (hinge, CH2 and CH3 domains)(italics). The point-mutated amino acids in the IgG2a Fc domain are shaded.

FIG. 5 shows proliferation to antigen upon re-stimulation. BALB/c mice were injected with control (white) or were vaccinated with Engerix-B™ (10 micrograms (mcg)) alone (light gray shading) or with a single dose of anti-TIM antibody (50 mcg)(dark gray shading). At the indicated times, the spleens were analyzed for proliferation to Hepatitis B surface antigen (96 h assay).

FIG. 6 shows the production of cytokines after re-stimulation with antigen. BALB/c mice were injected with control (white) or were immunized with 10 mcg of Hepatitis B vaccine (light gray shading), or with 10 mcg vaccine with anti-TIM-1 antibodies (dark gray shading). At days 7, 14, and 21, spleen cells were stimulated in vitro with Hepatitis B antigen. After 96 hours, the supernatants were analyzed for IFN-γ and IL-4 production, respectively.

FIG. 7 shows the production of hepatitis B specific antibodies. Serum samples from mice injected with control (PBS+alum:white) or vaccinated with Hepatitis B vaccine with (light gray shading) or without (dark gray shading) anti-TIM antibodies (single dose; 50 mcg) were tested by ELISA for the presence of antibodies specific for Hepatitis B surface antigen on day 7 after immunization.

FIG. 8 shows the proliferation of hepatitis B surface antigen-specific splenocytes in a dose dependent relationship with antigen stimulation. Splenocytes from mice vaccinated once with 10 mcg of Engerix B™, with or without 100 mcg TIM-1 monoclonal antibodies (mAbs), were isolated and cultured in the presence or absence of increasing hepatitis B surface antigen concentrations. After 4 days of incubation, the wells were analyzed for proliferation using the Delfia Cell Proliferation Assay. Mice that received vaccine with TIM-1 mAbs produced a statistically significantly higher proliferative response (p<0.05) against specific antigen versus vaccination with the Engerix B™ vaccine alone or with the isotype control antibody.

FIG. 9 shows the production of IFN-γ upon stimulation with specific antigen (hepatitis B surface antigen). Supernatants from the proliferation assay wells described above were removed for cytokine analysis by ELISA. Mice that received vaccine with TIM-1 mAbs produced a significantly higher amount of IFN-γ (p<0.05) in response to antigen stimulation than the mice that received vaccine alone or vaccine with the isotype control antibody. No IL-4 was detectable.

FIG. 10 shows that mice immunized with HIVp24 antigen plus TIM-1 mAb yielded a significantly higher proliferative response (p<0.05 compared to CpG) to antigen compared to either the isotype control antibody or CpG oligonucleotides. Mice were vaccinated subcutaneously with a single dose of HIVp24 antigen (25 mcg) in PBS and intraperitoneally with either 50 mcg TIM-1 mAb, isotype control antibody, or 50 mcg CpG (1826) oligodeoxy-nucleotides on days 1 and 15. Mice were then sacrificed on day 21 and the spleen cells were harvested for proliferation to antigen.

FIG. 11 shows the proliferative response of splenocytes to influenza antigen. BALB/c mice were immunized with 30 mcg of the influenza vaccine Fluvirin™ or Fluvirin™+anti-TIM-1 antibodies (single dose; 50 mcg antibody). Ten days later, the response to stimulation by virus (H1N1) was measured in a 96 h proliferation assay. PBS, and the anti-TIM-1 antibody alone were treatment controls. (n=4)

FIG. 12 shows cytokine production from influenza-immunized mice. BALB/c mice were immunized with 30 mcg of the influenza vaccine Fluvirin™ or Fluvirin™+anti-TIM antibodies (single dose; 50 mcg antibody). After 10 days, splenocytes were prepared and the production of Th1 (IFN-γ) and Th2 (IL-4) cytokines upon re-stimulation with virus (H1N1) was determined after 96 h in culture. (n=4)(N.D.=not determined) Mice given the vaccine plus TIM-1 antibody produced significantly higher amounts of IFN-γ in response to stimulation with inactivated influenza. No IL-4 was detected.

FIG. 13 demonstrates the cross-strain response after TIM-adjuvant treatment. The proliferative response of Beijing-immunized mice against stimulation by Beijing virus (A) or Kiev virus (B) were determined by the Delfia proliferation assay after 96 hours in culture. BALB/c mice were immunized with 10 mcg inactivated Beijing influenza virus in the presence or absence of 100 mcg TIM-1 mAb or isotype control (rat IgG2b). After 21 days, the spleens were harvested for in vitro analyses. Proliferation is enhanced using TIM-1 mAbs and response to Kiev stimulation demonstrates cross-strain immunity (p<0.01).

FIG. 14 shows the cross-strain cytokine response of Beijing-immunized mice against stimulation by Beijing virus (A) or Kiev virus (B). BALB/c mice were immunized with 10 mcg inactivated Beijing influenza virus in the presence or absence of 100 mcg TIM-1 mAb or isotype control (rat IgG2b). After 21 days, the spleens were harvested for in vitro analyses. Supernatants from the proliferation assays were analyzed for the presence of IFN-γ. Panel A shows that addition of TIM-1 mAbs significantly (p<0.01) enhances the production of IFN-γ in response to Beijing virus (H1N1) stimulation. Panel B shows that the addition TIM-1 mAbs also significantly (p<0.01) enhances the production of IFN-γ in response to stimulation with the heterosubtypic Kiev strain (H3N2).

FIG. 15 shows the IL-4 cytokine production of Beijing-immunized mice against stimulation by Beijing virus (A) or Kiev virus (B). BALB/c mice were immunized with 10 mcg inactivated Beijing influenza virus in the presence or absence of 100 mcg TIM-1 mAb or isotype control (rat IgG2b). After 21 days, the spleens were harvested for in vitro analyses. Supernatants from the proliferation assays were analyzed for the presence of IL-4. Panel A shows that addition of TIM-1 mAbs significantly (p<0.01) enhances the production of IL-4 in response to Beijing virus (H1N1) stimulation. Panel B shows that the addition TIM-1 mAbs also significantly (p<0.01) enhances the production of IL-4 in response to stimulation with the heterosubtypic Kiev strain (H3N2).

FIG. 16 shows the anti-rPA antibody response after vaccination. C57BL/6 mice were immunized with the 0.2 ml of AVA (Anthrax Vaccine Absorbed) BioThrax™ or BioThrax™+anti-TIM-1 antibodies. Seven days later, total serum antibodies specific for rPA were measured in an ELISA. BioThrax™ alone and BioThrax™+isotype matched antibody were treatment controls.

FIG. 17 shows anti-TIM adjuvant effects for anthrax vaccination. C57BL/6 mice were immunized with recombinant Protective Antigen (rPA; 40 mcg) or rPA+anti-TIM-3 antibodies (single dose; 50 mcg). Ten days later, the response of splenocytes to re-stimulation by rPA was measured in a 96 h proliferation assay. PBS and rPA+isotype matched control antibody were treatment controls.

FIG. 18 shows an exemplary TIM expression vector.

FIG. 19 shows that TIM-3 signaling accelerates diabetes in mice, as described in Sanchez-Fueyo et al., Nat. Immunol. 4:1093-1101 (2003)(figure adapted from Sanchez-Fueyo et al.).

FIG. 20 shows that delivering anti-TIM-1 antibodies with vaccination elicits complete tumor rejection.

FIG. 21 shows that vaccines supplemented with anti-TIM-1 antibodies greatly inhibit tumor growth upon challenge with live tumor cells.

FIG. 22 shows that vaccines supplemented with anti-TIM-1 antibodies greatly inhibit tumor growth upon challenge with live tumor cells.

FIG. 23 shows that pre-treatment of animals with anti-TIM-1 antibody prior to live tumor cell challenge significantly restrains tumor growth.

FIG. 24 shows that pre-treatment of animals with anti-TIM-1 antibody prior to live tumor cell challenge significantly limits tumor growth.

FIG. 25 shows that anti-TIM-1 antibody is effective as a cancer vaccine adjuvant. In this study, C57BL/6 mice were vaccinated against EL4 thymoma tumors, using gamma-irradiated EL4 cells as a source of antigen, and either anti-TIM-1 antibody or rIgG2b isotype control. These animals were boosted twice after initial vaccination and were subsequently challenged with a subcutaneous injection of live EL4 tumor cells. Throughout the post-challenge observation period, the mean tumor size of mice receiving anti-TIM-1 antibody as a tumor vaccine adjuvant was less than that of mice receiving the isotype control antibody. In addition, nineteen days after live tumor challenge, four of the eight animals receiving anti-TIM-1 antibody had fully rejected tumor, while no tumor rejection was observed among the eight mice receiving isotype control antibody.

FIG. 26 shows that vaccination with anti-TIM-1 adjuvants drives the generation of protective immunity. Splenocytes were recovered from mice which were first vaccinated against EL4 thymoma using anti-TIM-1 as a tumor vaccine adjuvant, and had also completely rejected subsequent live tumor challenge. After red blood cell depletion in vitro, 10exp7 splenocytes were adoptively transferred into naive C57BL/6 mouse recipients. Other mice received adoptive transfer of splenocytes harvested from either naive mice or mice receiving rIgG2a during tumor vaccination and boosting. One day after transfer, all recipient mice were challenged with subcutaneous injection of 10exp6 live EL4 tumor cells. Splenocytes transferred from mice receiving anti-TIM-1 antibody as a tumor vaccine adjuvant were able to confer protection against subsequent tumor challenge in recipient mice. This protection was not achievable when splenocytes from either naive mice, nor mice vaccinated with gamma-irradiated EL4 plus rIgG2a were transferred. These results demonstrate establishment of a durable and transferable immunity against tumor when vaccination is accomplished using an anti-TIM-1 antibody adjuvant.

FIG. 27 shows that anti-TIM-1 therapy is effective in preventing tumor growth. Anti-TIM-1 antibody is effective as a stand-alone therapeutic agent capable of slowing growth of previously established EL4 thymoma tumors. In this study, naive C57BL/6 mice were challenged with subcutaneous injection of 10exp6 live EL4 tumor cells, then treated six days later by intraperitoneal injection of 100 mcg anti-TIM-1 antibody, or 100 mcg rIgG2a control antibody. Following tumor growth after the onset of treatment, a statistically significant restraint of tumor growth was observed 15 days after antibody delivery into anti-TIM-1 treated mice. The results demonstrate a capacity for anti-TIM-1 antibody to limit tumor growth as a therapeutic after establishment of the tumor.

FIG. 28 shows that TIM-3-specific antibody reduces tumor growth when used as a vaccine adjuvant. In order to evaluate the potential adjuvant effects of TIM-3-specific antibody, mice were vaccinated against EL4 thymoma tumors using gamma-irradiated EL4 cells as a source of antigen, and either anti-TIM-3 antibody or rIgG2a isotype control. These animals were boosted once after initial vaccination and were subsequently challenged with a subcutaneous injection of live EL4 tumor cells. Over time, the mean size of challenge tumors in mice that received anti-TIM-3 antibody as a tumor vaccine adjuvant was less than that of mice receiving the isotype control antibody.

FIG. 29 shows that anti-TIM-3 antibody is effective as a stand-alone therapeutic agent capable of slowing growth of previously established EL4 thymoma tumors. In this study, naive C57BL/6 mice were challenged by subcutaneous injection of 10exp6 live EL4 tumor cells, then treated nine days later with the first of three weekly intraperitoneal injections of 100 mcg anti-TIM-3 antibody, or 100 mcg rIgG2a isotype control antibody. Following tumor growth after the onset of treatment, restrained progression was identified in anti-TIM-3 treated mice within one week of initial dosing. This effect continued over time, developing into a statistically significant restraint of tumor growth through day 17. The results demonstrate a capacity for anti-TIM-3 antibody to limit tumor growth of pre-established tumors.

FIG. 30 shows exemplary diseases, the relationship to Th1/Th2 responses, and desired shifts in amounts of Th1 and Th2 using a composition of the invention containing a TIM targeting molecule.

FIG. 31 shows the cDNA sequence (SEQ ID NO:6) of mouse TIM-2 from BALB/c mouse. The cDNA sequence includes the signal sequence, Ig, mucin, transmembrane and intracellular domains.

FIG. 32 shows the nucleotide and amino acid sequences of various mouse and human TIM molecules, as described in WO 03/002722. The sequences shown are mouse TIM-1 BALB/c allele (amino acid and nucleotide sequences SEQ ID NOS:7 and 8, respectively); mouse TIM-1 C.D2 ES-HBA and DBA/2J allele (amino acid and nucleotide sequences SEQ ID NOS:9 and 10, respectively); mouse TIM-2 BALB/c allele (amino acid and nucleotide sequences SEQ ID NOS:11 and 12, respectively); mouse TIM-2 C.D2 ES-HBA and DBA/2J allele (amino acid and nucleotide sequences SEQ ID NOS:13 and 14, respectively); mouse TIM-3 BALB/c allele (amino acid and nucleotide sequences SEQ ID NOS:15 and 16, respectively); mouse TIM-3.C.D2 ES-HBA and DBA/2J allele (amino acid and nucleotide sequences SEQ ID NOS:17 and 18, respectively); TIM-4 BALB/c allele (amino acid and nucleotide sequences SEQ ID NOS:19 and 20, respectively); TIM-4 mouse C.D2 ES-HBA and DBA/2J (amino acid and nucleotide sequences SEQ ID NOS:21 and 22, respectively); human TIM-1 allele 1 (amino acid and nucleotide sequences SEQ ID NOS:23 and 24, respectively); human TIM-1, allele 2 (amino acid and nucleotide sequences SEQ ID NOS:25 and 26, respectively); human TIM-1 allele 3 (amino acid and nucleotide sequences SEQ ID NOS:27 and 28, respectively); human TIM-1 allele 4 (amino acid and nucleotide sequences SEQ ID NOS:29 and 30, respectively); human TIM-1 allele 5 (amino acid and nucleotide sequences SEQ ID NOS:31 and 32, respectively); human TIM-1 allele 6 (amino acid and nucleotide sequences SEQ ID NOS:33 and 34, respectively); human TIM-3 allele 1 (amino acid and nucleotide sequences SEQ ID NOS:35 and 36, respectively); human TIM-3 allele 2 (amino acid and nucleotide sequences SEQ ID NOS:37 and 38, respectively); human TIM-4 allele 1 (amino acid and nucleotide sequences SEQ ID NOS:39 and 40, respectively); human TIM-4 allele 2 (amino acid and nucleotide sequences SEQ ID NOS:41 and 42, respectively).

FIG. 33 shows that the mouse renal adenocarcinoma cell line RAG expresses TIM-1 on its cell surface. TIM-1 antibodies (filled) specifically bind to RAG cells, as compared to unstained controls or cells stained with control antibodies (open).

FIG. 34 shows that the human renal adenocarcinoma cell line 769-P expresses TIM-1 on its cell surface. TIM-1 antibodies (filled) specifically bind to 769-P cells, as compared to unstained controls or cells stained with control antibodies (open).

FIG. 35 shows that the mouse tumor cell lines EL4 (a thymoma) and 11PO-1 (a transformed mast cell) express TIM-3 on their cell surface. TIM-3 antibodies (filled) specifically bind to the respective tumor cells, as compared to unstained controls or cells stained with control antibodies (open).

FIG. 36 shows a summary of mouse tumor cell lines tested for expression of TIM-3 and TIM-3 ligand (TIM-3L). Both TIM-3 and TIM-3 ligand expressing tumor cell lines were identified. TIM-3 expression was monitored using TIM-3 monoclonal antibodies. TIM-3 ligand expression was demonstrated by measuring specific binding of TIM-3/Fc fusion protein to the respective cells.

### DETAILED DESCRIPTION OF THE INVENTION

The present invention provides compositions containing an antigen and a TIM targeting molecule and methods of using such compositions. In one embodiment, the invention provides a method of stimulating an immune response in an individual by administering a composition comprising an antigen and a TIM targeting molecule in a pharmaceutically acceptable carrier. In another embodiment, the invention provides a method of stimulating an immune response in an individual by administering an antigen and a TIM targeting molecule, which can be administered together in a single composition or separately. The compositions and methods of the invention can be used to target TIM signaling, thereby modulating levels of Th1 and Th2 helper cells. The compositions and methods of the invention can be used advantageously to modulate the levels of Th1 and Th2 to increase an appropriate and more effective immune response.

Vaccination protocols against infectious pathogens are often hampered by poor vaccine immunogenicity, an inappropriate type of response (antibody versus cell-mediated immunity), lack of long-term memory and/or failure to generate immunity against different serotypes of a given pathogen. Adjuvants, such as aluminum salts have been used in vaccine formulations for over 70 years and their safety and efficacy for certain indications is well established (Baylor et al., Vaccine 20 Suppl 3, S18-23 (2002)). One potential drawback to the use of aluminum salts as vaccine adjuvants for intracellular pathogens is the induction of IgG1 and IgE antibody responses. Furthermore, aluminum salts fail to stimulate Th1 immunity and do not promote the induction of CD8⁺ T cells (Newman et al. J. Immunol. 148:2357-2362 (1992); Sheikh et al. Vaccine 17:2974-2982 (1999)). To date there are no adjuvants or biologicals that can alter the Th1/Th2 balance at will. No vaccines containing adjuvants other than aluminum salts have been licensed in the U.S.

Recently, a new family of molecules, now called TIMs (T cell Immunoglobulin and Mucin), that play an important role in regulating the responses of activated Th1 or Th2 T helper cells has been characterized (Monney et al. Nature 415:536-541 (2002); McIntire et al. Nat. Immunol. 2:1109-1116 (2001)). Specifically, TIM-3 has been identified as a cell surface molecule that is expressed on terminally differentiated Th1 cells. In contrast, TIM-1 is expressed on differentiated Th2 cells (Kuchroo et al. Nat. Rev. Immunol. 3:454-462 (2003)). The invention provides the use of anti-TIM antibodies and TIM fusion proteins, for example, consisting of the extracellular TIM domains fused with an immunoglobulin Fc domains (TIM/Fc), as vaccine adjuvants and stimulators to enhance immune responses. The molecules of the invention can be used as vaccine adjuvants for the treatment of infectious diseases and for the treatment of malignancies, such as tumors.

Protection against infectious agents requires the induction of specific adaptive immune responses against the pathogenic organism. The effector phase of adaptive immune responses is critically influenced by the maturation of CD4⁺ T helper cells into either Th1 or Th2 subtypes. Each subtype secretes cytokines that promote distinct immunological effects that are opposed to one another and that cross-regulate each other's expansion and function. Th1 cells secrete high amounts of cytokines such as interferon-gamma (IFN-γ), tumor necrosis factor-alpha (TNF-α), interleukin-2 (IL-2) and IL-12, and low amounts of IL-4 (Mosmann et al., J. Immunol. 136:2348-2357 (1986)). Th1-associated cytokines promote CD8⁺ cytotoxic T lymphocyte (CTL) activity and, in mice, IgG2a antibodies that effectively lyse cells infected with intracellular pathogens (Allan et al., J. Immunol. 144:3980-3986 (1990). In contrast, Th2 cells secrete high amounts of cytokines such as IL-4, IL-13 and IL-10, but low IFN-γ and promote antibody responses, in mice, generally of the IgG1 non-lytic isotype. Th2 responses are particularly relevant for humoral responses, such as in protection from anthrax (Leppla et al., J. Clin. Invest. 110:141-144 (2002)) and for the elimination of helminthic infections (Yoshida et al., Parasitol. Int. 48:73-79 (1999)).

Whether a resulting immune response is Th1- or Th2-driven largely depends on the pathogen involved and on factors in the cellular environment, such as cytokines. Failure to activate a T helper response, or the correct T helper subset, can result not only in the inability to mount a sufficient response to combat a particular pathogen, but also in the generation of poor immunity against re-infection. Many infectious agents are intracellular pathogens in which cell-mediated responses, as exemplified by Th1 immunity, would be expected to play an important role in protection and/or therapy. Moreover, induction of inappropriate Th2 responses negatively affects disease outcome against intracellular pathogens such as M. tuberculosis (Lindblad et al., Infect. Immun. 65:623-629 (1997)) or Leishmania, or S. mansoni (Scott et al., Immunol. Rev. 112:161-182 (1989)). Nonhealing forms of human and murine leishmaniasis result from strong but counterproductive Th2-like-dominated immune responses. Lepromatous leprosy also appears to feature a prevalent, but inappropriate, Th2-like response. HIV infection represents another example. Here, it has been suggested that a drop in the ratio of Th1-like cells to other Th cell subpopulations can play a critical role in the progression toward disease symptoms.

The clearance of many viral infections relies on the function of CD8⁺ T cells, which in turn are enhanced by a Th1-priming cytokine environment. Furthermore, a Th1 response against one virus serotype is required in order to be able to induce protective immunity against a virus of a different serotype, a phenomenon known as heterosubtypic immunity. Current vaccination strategies target the elicitation of antibodies specific for a given viral serotype. A disadvantage to this strategy, however, is that antibodies are very specific and give no protection to viruses of different serotypes which arise from changes in surface protein amino acid sequences of, in the example of influenza, hemagglutinin and neuraminidase. These mutations may be minor (antigenic drift) or major (antigenic shift). For many common viral pathogens, efforts must be made on a recurring basis to monitor which serotypes are prevalent around the world. An example of this is the annual monitoring of emerging influenza serotypes, which are anticipated to be the major infectious strains. The failure to induce heterosubtypic immunity has also been observed in a mouse model of influenza. In this model, use of an inactivated viral vaccine does not promote a Th1 profile. This renders the mice incapable of efficient viral clearance and susceptible to re-infection with a serologically distinct virus (Moran et al., J. Infect. Dis. 180:579-585 (1999)). In contrast, mice treated with IL-12 and anti-IL-4 antibodies in conjunction with inactivated virus during the vaccination generated an immune response characterized by the production of Th1 cytokines. These mice are able to mount a heterosubtypic cellular immune response to a subsequent challenge with a serologically different virus. Taken together with what is known about Th1/Th2 priming environments, the data suggest that T helper stimulation and/or deviation toward a Th1 cytokine response may generate broad immunity against various serotypes resulting from either antigenic drift or antigenic shift. Thus, TIM-mediated induction of a Th1 response can be a viable strategy for improving current vaccines and TIM targeting reagents, such as TIM proteins or TIM antibodies, can be used to stimulate cross strain or heterosubtypic immunity.

Aluminum salts have been used as relatively safe and effective vaccine adjuvants to enhance antibody responses to certain pathogens. One of the disadvantages of such adjuvants is that they are relatively ineffective at stimulating a cell-mediated immune response (Grun and Maurer, Cell Immunol. 121:134-145 (1989)). The development of other adjuvants with low toxicity and/or the ability to precisely control and stimulate cellular immunity has remained a challenge. To increase the effectiveness of an adaptive immune response, such as in a vaccination protocol or during a microbial infection, the invention provides the use of agents that target the TIM-1, -2, -3, or -4 signaling pathway as adjuvants that are effective in protecting the host.

Vaccination protocols to stimulate responses of the immune system can be used for the prevention and treatment of infectious diseases, such as infections caused by, for example, viral, parasitic, bacterial, archaebacterial, mycoplasma, and prion agents. Vaccination protocols can also be used for the prevention and treatment of hyperplasias and malignancies, such as tumors, and for any other disease in which stimulation of the immune system is beneficial as a preventative or therapeutic measure. Examples of such other diseases include autoimmune diseases, for example, multiple sclerosis, rheumatoid arthritis, type 1 diabetes, psoriasis, and other autoimmune diseases. One of the properties of autoimmune diseases is the generation of autoreactive antibodies against self-epitopes. Such autoreactive antibodies play a very important role in the development, progression and chronic nature of autoimmune diseases. Vaccines can be used that, for example, lead to the generation of anti-idiotypic antibodies that neutralize such autoreactive antibodies.

As disclosed herein, reagents targeting the TIM-1 signaling pathways serve as effective vaccine adjuvants (see Examples). Such reagents include antibodies against TIM-1, antibodies against TIM-1 ligands, recombinant TIM-1 proteins including TIM-1 fusion proteins, and TIM-1 ligand proteins including TIM-1 ligand fusion proteins. Thus, the invention provides TIM-1 targeting molecules that function as effective vaccine adjuvants. The invention additionally provides similar types of molecules that target other TIMs, including but not limited to TIM-3, as well as TIM-2 and TIM-4.

The invention provides agents which target the TIM signaling pathways and serve as effective vaccine adjuvants. As used herein, the term “agent,” when used in reference to the TIM signaling pathway, refers to a molecule that modulates a signaling pathway mediated by a TIM. A TIM targeting agent is also referred to herein as a TIM targeting molecule or reagent. Such agents include, as exemplified for TIM-1, antibodies against TIM-1, antibodies against TIM-1 ligands, recombinant TIM-1 proteins including TIM-1 fusion proteins, and TIM-1 ligand proteins including TIM-1 ligand fusion proteins. Similar types of agents can be used to modulate other respective TIM signaling pathways, including TIM-2, -3 or -4. Fusion proteins include, for example, fusions of TIM-1 or TIM-1 ligands with proteins or protein fragments, such as with the Fc region of immunoglobulins, with albumin, with transferrin, with a Myc tag, with a polyhistidine tag or other desired proteins or protein fragments. Agents of the invention also include chemically modified agents, such as pegylated TIM or TIM ligands or other desired chemical modifications. It is understood that, when referring to a particular TIM, polymorphic and splice variants of that TIM are included. An agent of the invention can also be a small molecule, a peptide, a polypeptide, a polynucleotide, including antisense and siRNAs, a carbohydrate including a polysaccharide, a lipid, a drug, as well as mimetics, derivatives and combinations thereof that stimulate or inhibit interaction of a specific TIM, for example, TIM-1, -2, -3, or -4, with its ligands, or stimulate or inhibit TIM or TIM ligand signaling. It is understood that any description herein for the use of agents that target the TIM-1 signaling pathway are exemplary and can similarly be applied to agents that target other TIM signaling pathways, including TIM-2, TIM-3 and TIM-4. The agents of the invention can be used as adjuvants to stimulate the body's immune response, such as in a vaccination. The use of these agents as adjuvants is not limited to any specific type of immunostimulatory treatment or vaccination and can include, but is not limited to, any of the above examples of vaccination protocols.

The invention provides a composition comprising an antigen and a TIM targeting molecule or agent in a pharmaceutically acceptable carrier. As used herein, a “TIM targeting molecule” refers to a molecule that binds to a TIM or TIM ligand. Exemplary TIM targeting molecules include, but are not limited to, antibodies against a TIM, antibodies against a TIM ligand, a recombinant TIM protein, a TIM fusion polypeptide, a TIM ligand, including a TIM ligand fusion polypeptide. As disclosed herein, an antigen and TIM targeting molecule or agent can be administered in a single composition or as separate compositions.

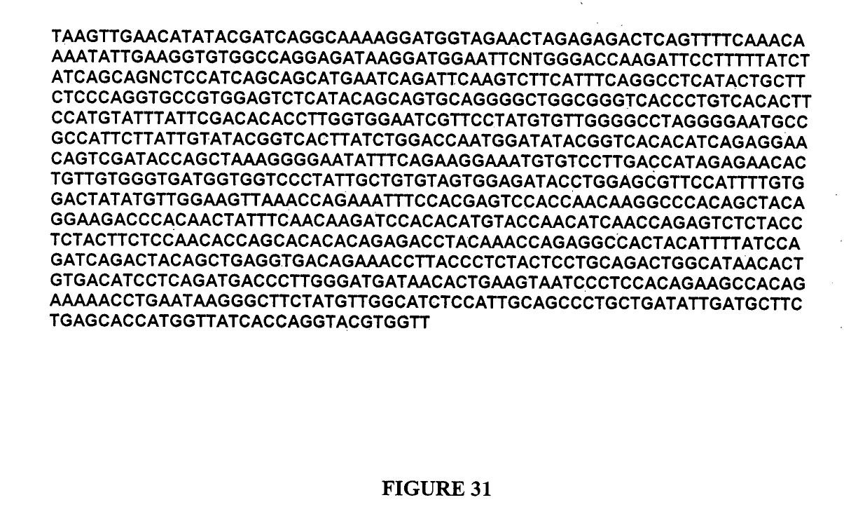

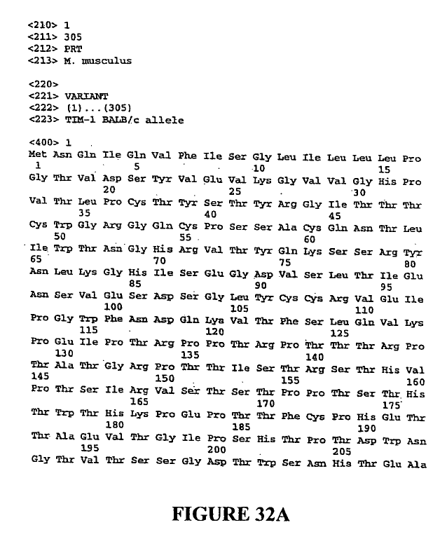

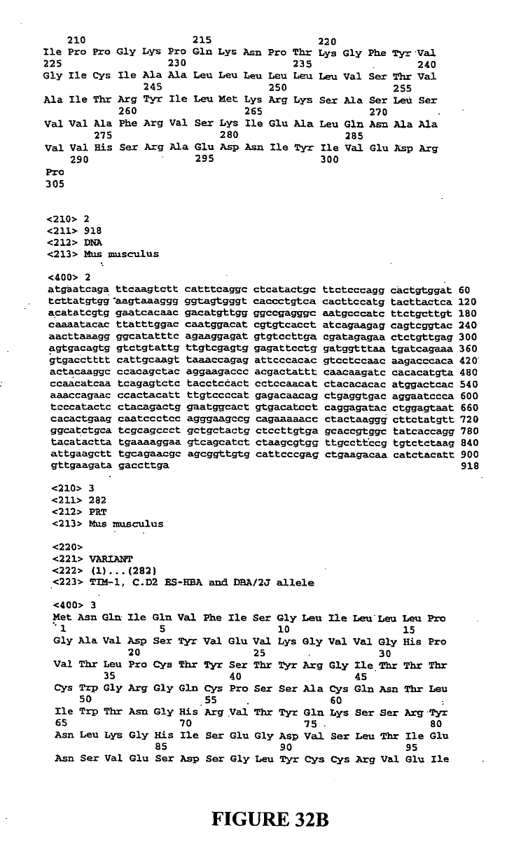

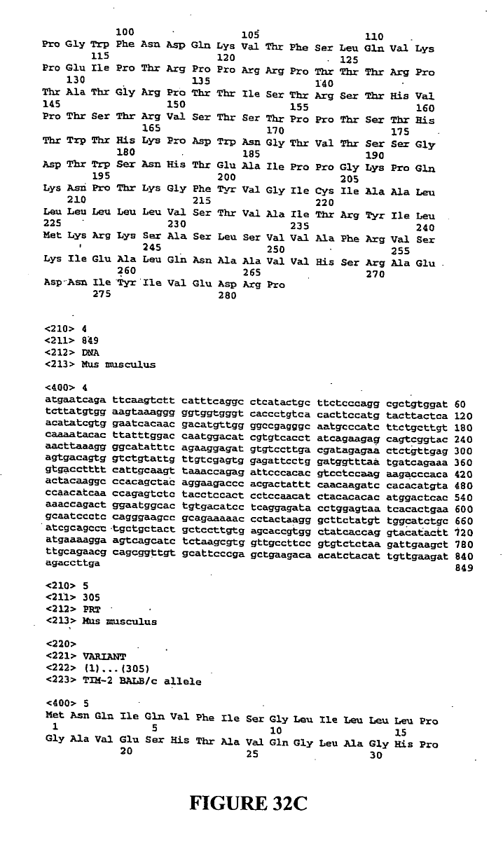

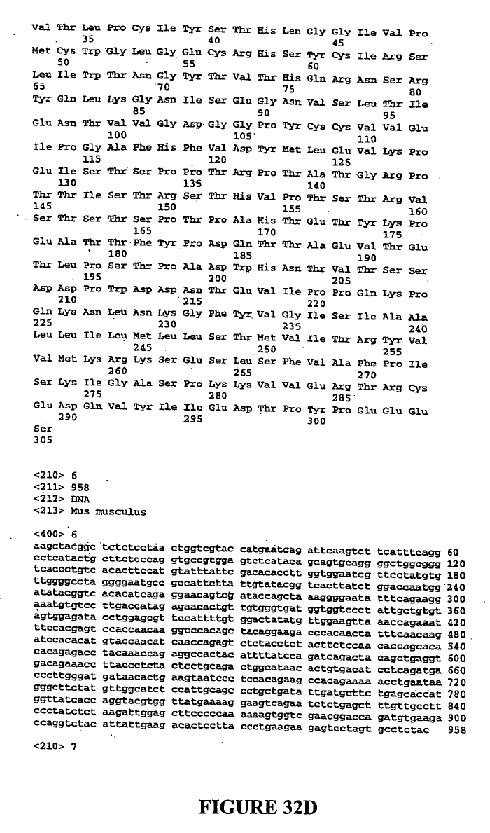

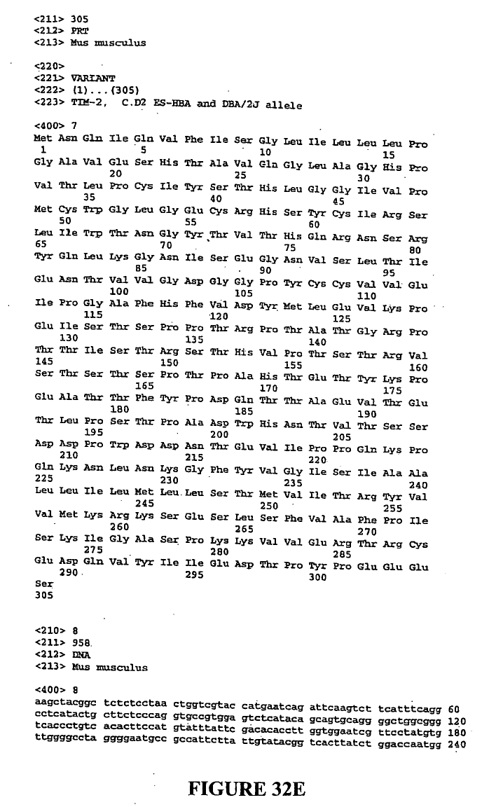

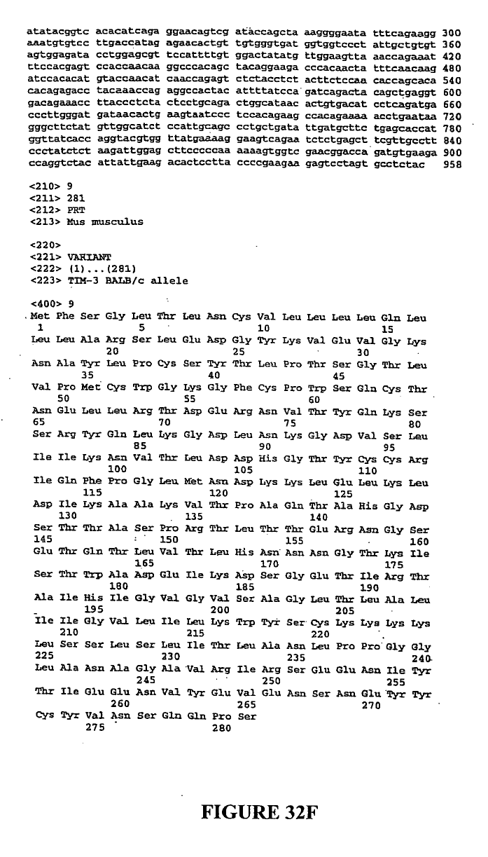

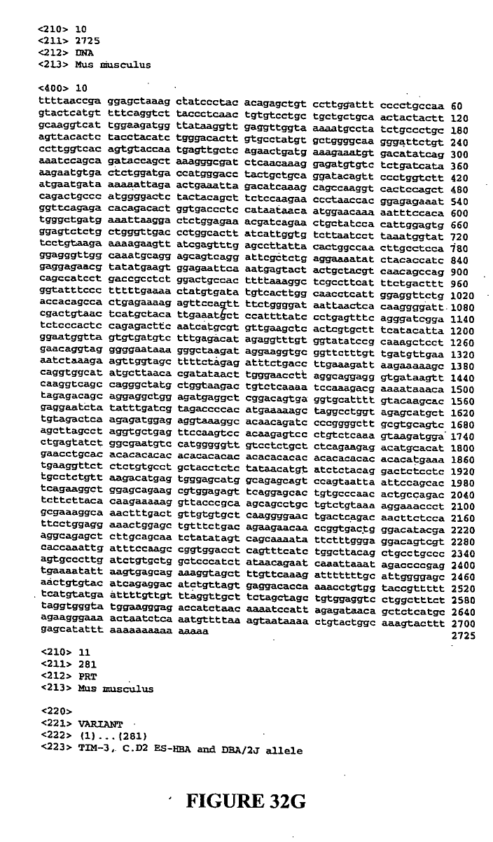

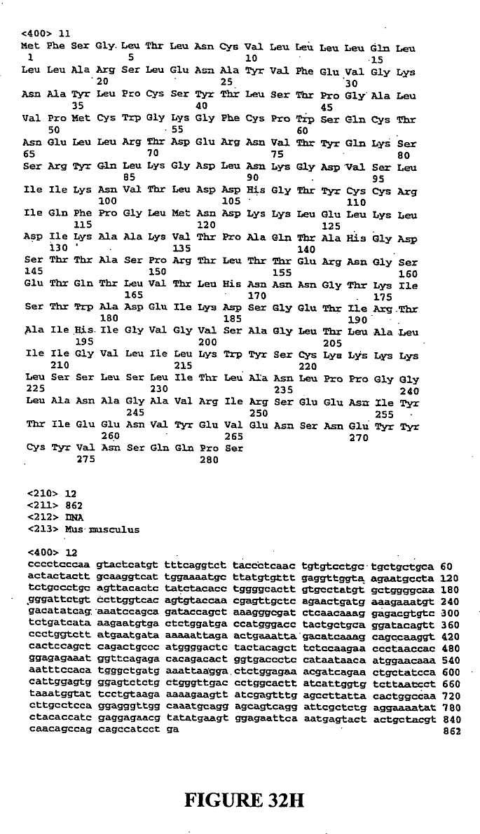

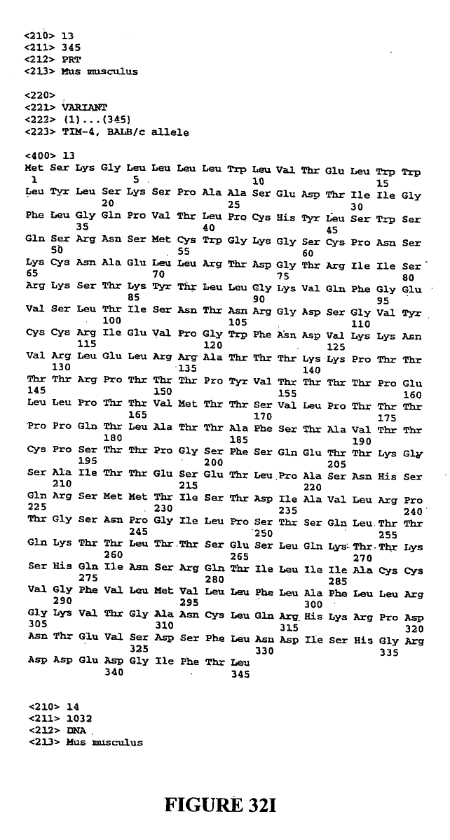

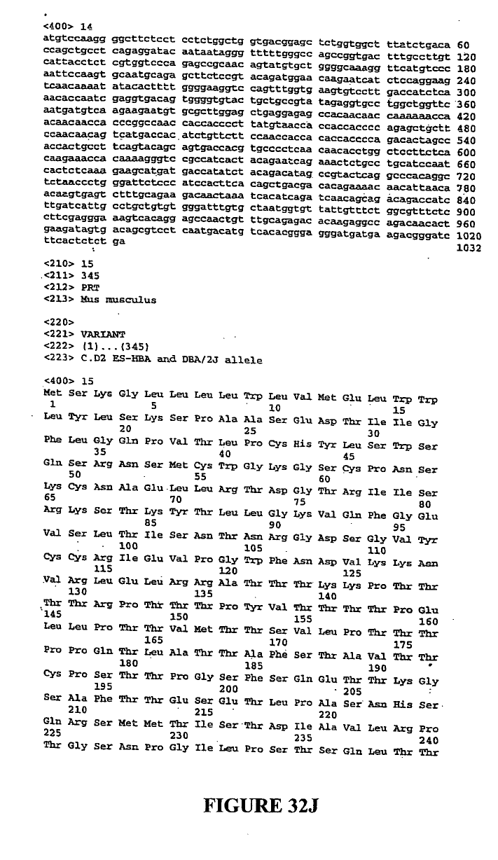

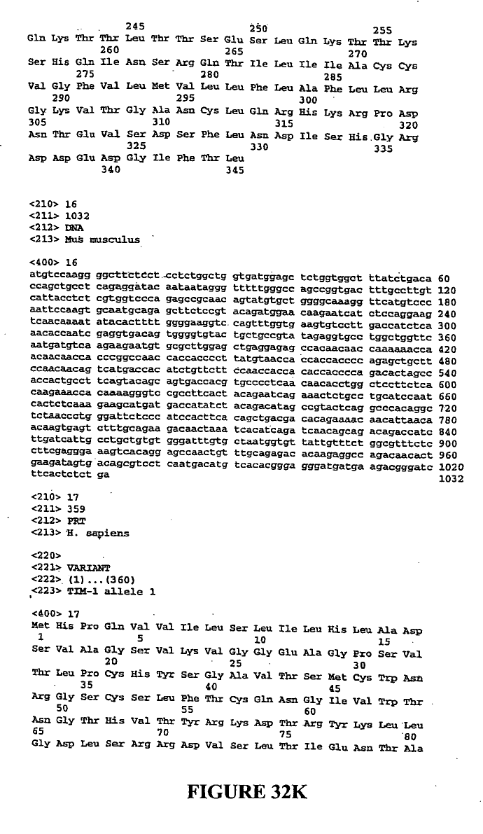

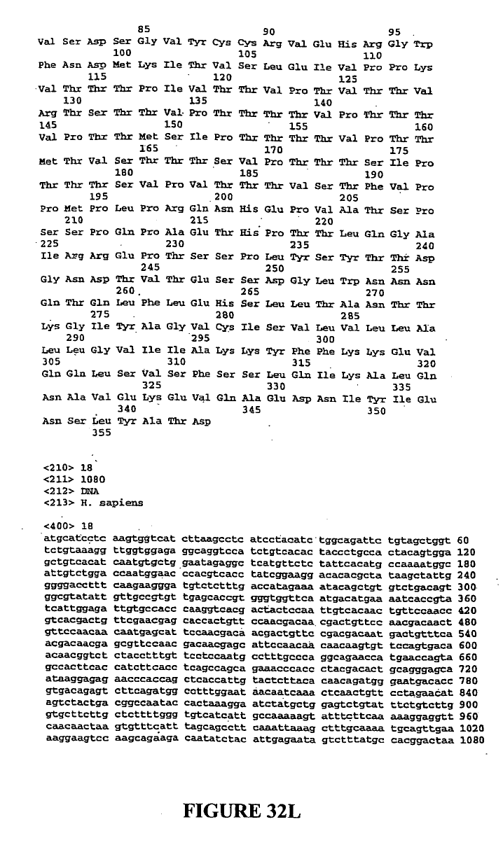

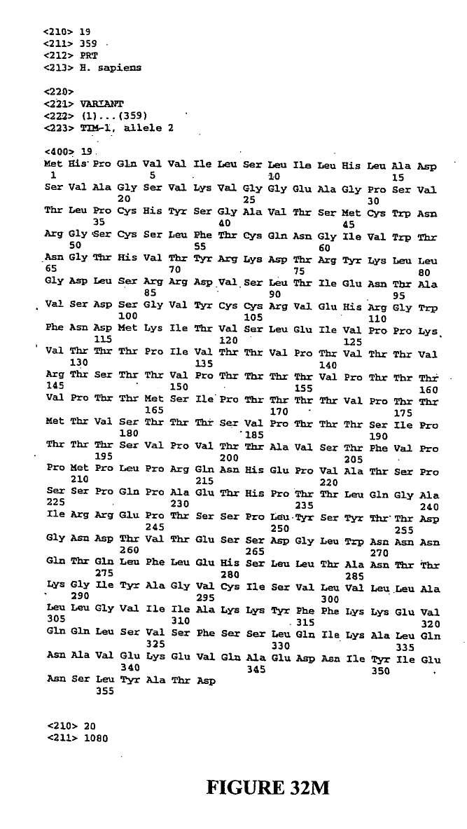

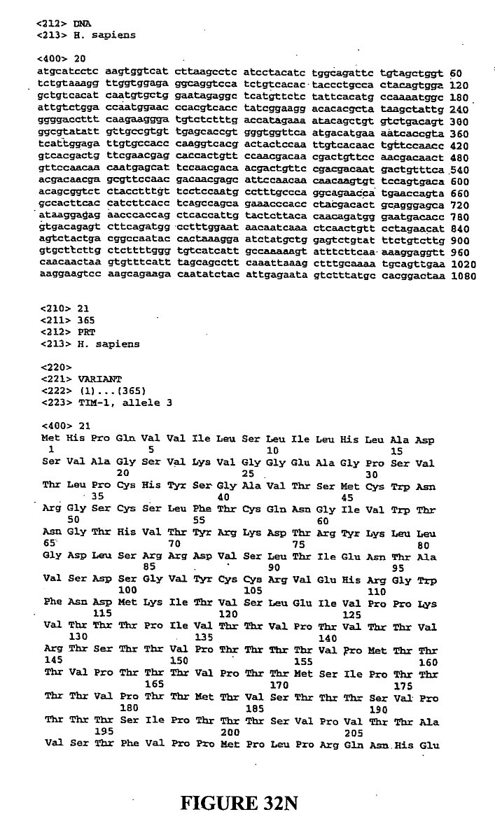

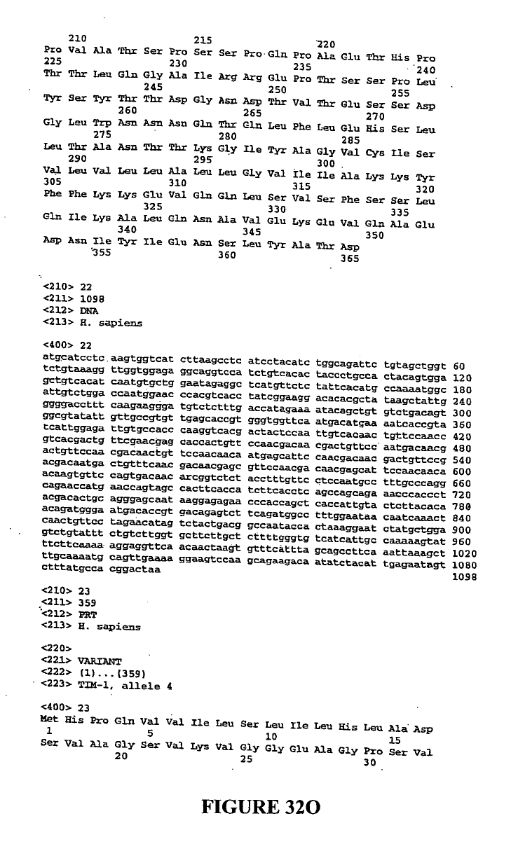

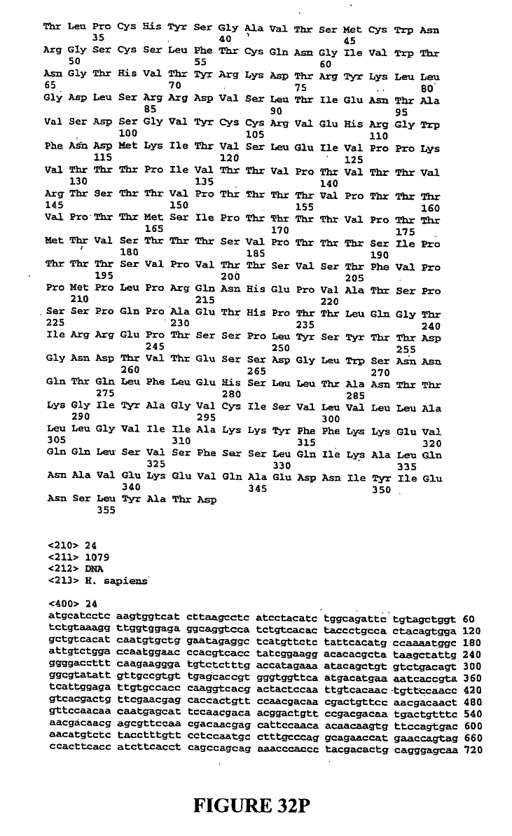

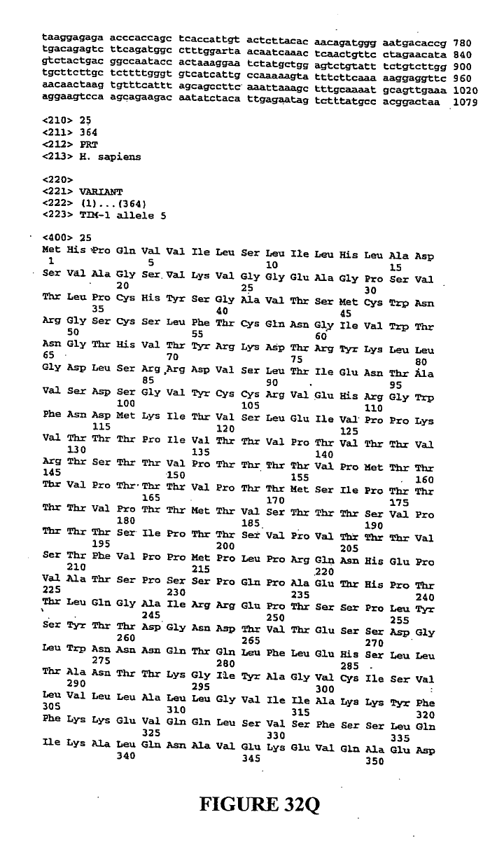

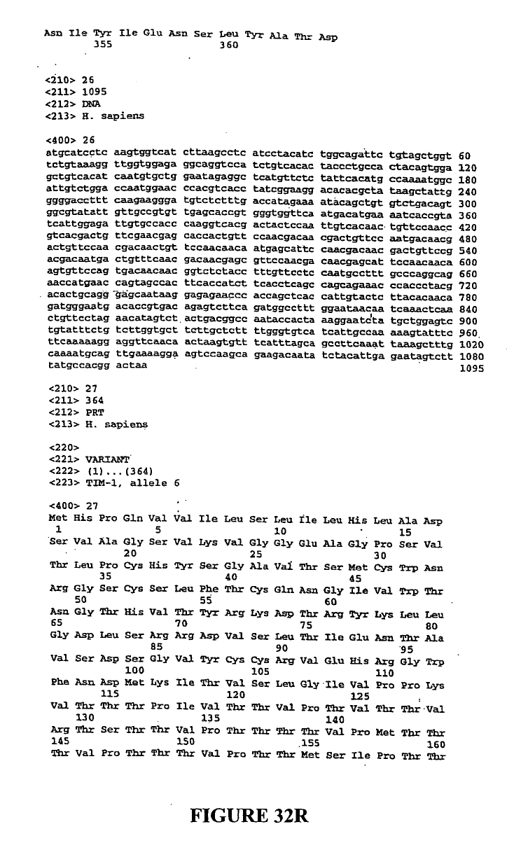

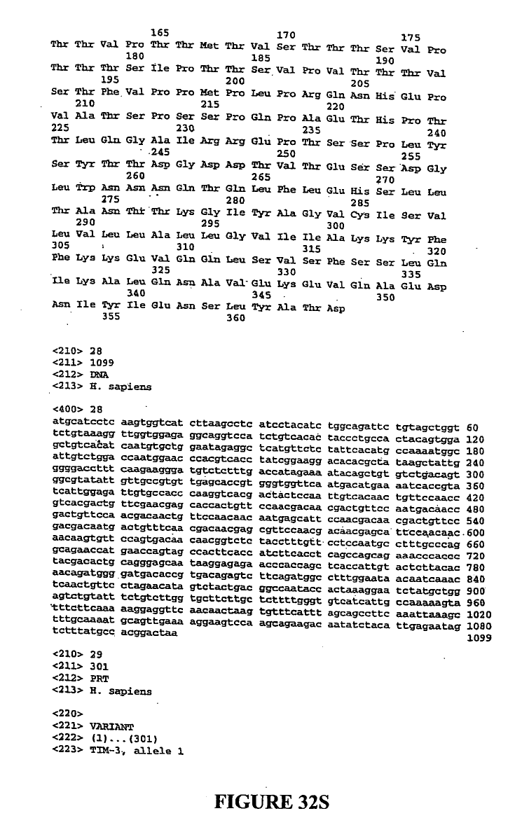

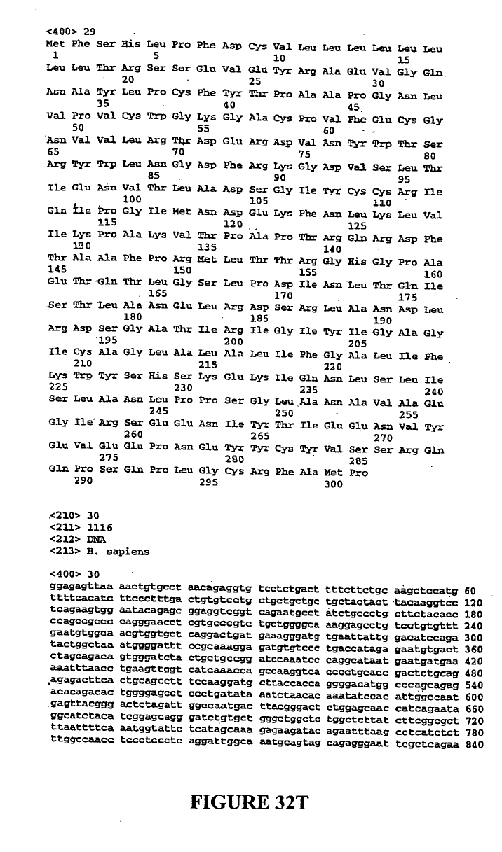

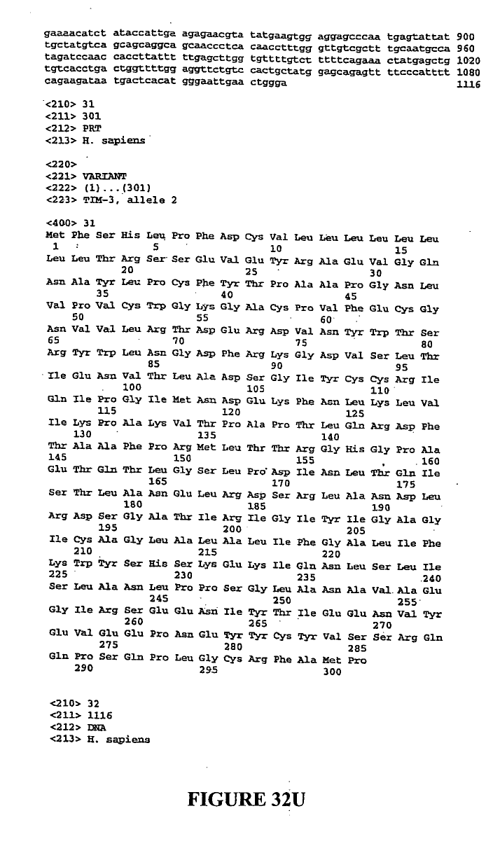

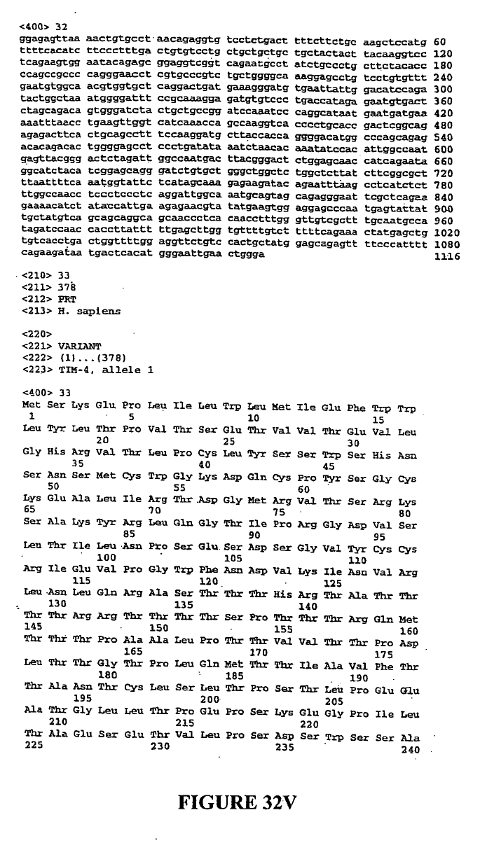

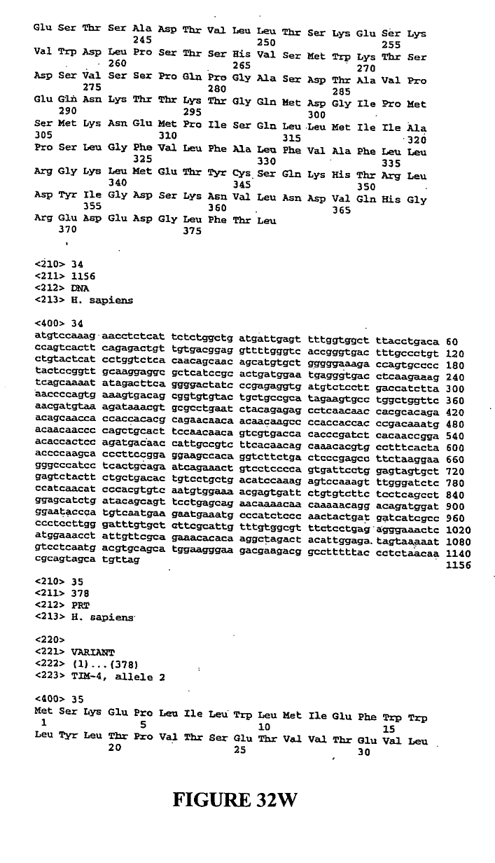

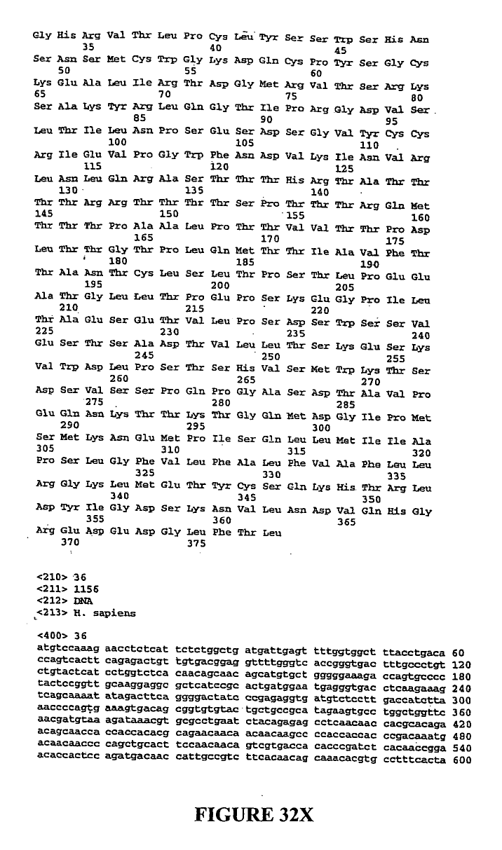

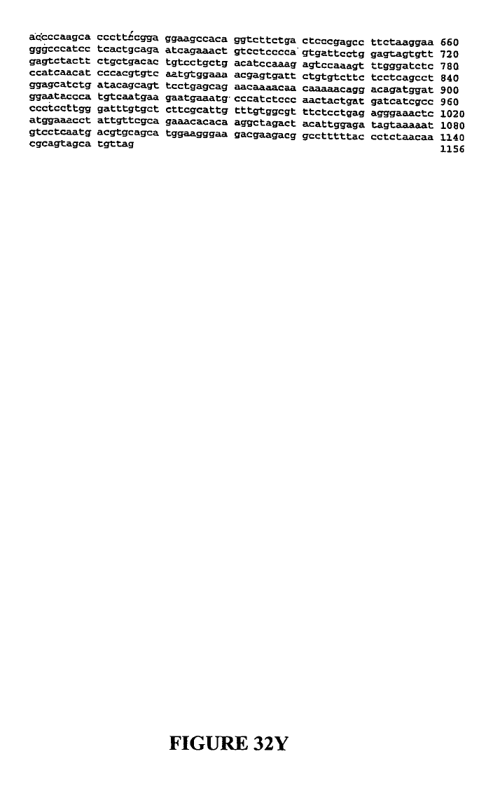

Various TIMs are well known to those skilled in the art, including TIM-1, TIM-2, TIM-3 and TIM-4. Various TIMs are taught, for example, in WO 03/002722; WO 97/44460; U.S. Pat. No. 5,622,861, issued Apr. 22, 1997; and U.S. publication 2003/0124114, each of which is incorporated herein by reference. Exemplary TIM sequences are shown in FIGS. 31 and 32. A variety of TIMs from different species can be used in compositions and methods of the invention, depending on the desired use. A TIM from a particular species can be used for a particular use, for example, a human TIM can be used in a human, if desired. TIMs from other species can also be used, as desired.

In one embodiment, a TIM targeting molecule can be, for example a fusion protein with a TIM, for example, TIM-1, TIM-2, TIM-3 or TIM-4, and can include at least one domain or portion thereof of an extracellular region of the TIM and a constant heavy chain or portion thereof of an immunoglobulin. In a particular embodiment, a soluble TIM fusion protein refers to a fusion protein that includes at least one domain of an extracellular domain of a TIM and another polypeptide. In one embodiment, the soluble TIM can be a fusion protein including the extracellular region of a TIM covalently linked, for example, via a peptide bond, to an Fc fragment of an immunoglobulin such as IgG; such a fusion protein typically is a homodimer. In another embodiment, the soluble TIM fusion can be a fusion protein including just the Ig domain of the extracellular region of a TIM covalently linked, for example, via a peptide bond, to an Fc fragment of an immunoglobulin such as IgG; such a fusion protein typically is a homodimer. As is well known in the art, an Fc fragment is a homodimer of two partial constant heavy chains. Each constant heavy chain includes at least a CH1 domain, the hinge, and CH2 and CH3 domains. Each monomer of such an Fc fusion protein includes an extracellular region of a TIM linked to a constant heavy chain or portion thereof (for example, hinge, CH2, CH3 domains) of an immunoglobulin. The constant heavy chain in certain embodiments can include part or all of the CH1 domain that is N-terminal to the hinge region of immunoglobulin. In other embodiments, the constant heavy chain can include the hinge but not the CH1 domain. In yet another embodiment, the constant heavy chain will exclude the hinge and the CH1 domain, for example, it will include only the CH2 and CH3 domains of IgG.

In one embodiment, the TIM targeting molecule can be a TIM antibody, for example, an antibody specific for TIM-1, TIM-2, TIM-3, or TIM-4. Antibodies to other TIMs can also be used. In another embodiment, the TIM targeting molecule is a TIM-Fc fusion polypeptide, for example, a TIM-1, TIM-2, TIM-3 or TIM-4 fused to an Fc. One skilled in the art can readily make a variety of TIM fusion polypeptides to an Fc or other desired polypeptide, including TIM polypeptide fragments containing desired domains. In yet another embodiment, the TIM targeting molecule or agent of the invention can be a small molecule, a peptide, a polypeptide, a polynucleotide, including antisense and siRNAs, a carbohydrate including a polysaccharide, a lipid, a drug, as well as mimetics, derivatives and combinations thereof that stimulates or inhibits TIM interaction with its ligands or TIM or TIM ligand signaling.

Targeting occurs when an agent or TIM targeting molecule directly or indirectly binds to, or otherwise interacts with, a TIM or TIM ligand or a component of a TIM or TIM ligand signaling pathway in a way that affects the activity of the TIM or TIM ligand. Activity can be assessed by those of ordinary skill in the art and with routine laboratory methods (see, for example, Reith, Protein Kinase Protocols Humana Press, Totowa N.J. (2001); Hardie, Protein Phosphorylation: A Practical Approach second ed., Oxford University Press, Oxford, United Kingdom (1999); Kendall and Hill, Signal Transduction Protocols: Methods in Molecular Biology Vol. 41, Humana Press, Totowa N.J. (1995)). For example, one can assess the strength of signal transduction or another downstream biological event that occurs, or would normally occur, following receptor binding. The activity generated by an agent that targets a TIM or TIM ligand can be, but is not necessarily, different from the activity generated when a naturally occurring TIM or TIM ligand binds a naturally occurring TIM or TIM ligand. For example, an agent or TIM targeting molecule that targets TIM-1 falls within the scope of the invention if that agent generates substantially the same activity that would occur had the receptor been bound by naturally occurring TIM-1 ligand. In addition, an agent or TIM targeting molecule can be an antagonist that inhibits signaling by a naturally occurring TIM ligand.

As described above, agents of the invention can contain two functional moieties: a targeting moiety that targets the agent to a TIM or TIM ligand-bearing cell (such as TIM-1, TIM-2, TIM-3 or TIM-4) and, for example, a dimerizing and/or target-cell depleting moiety that, for example, lyses or otherwise leads to the elimination of the TIM or TIM ligand-bearing cell, as discussed herein. Thus, the agent can be a chimeric polypeptide that includes a TIM polypeptide and a heterologous polypeptide such as the Fc region of the IgG and IgM subclasses of antibodies. The Fc region may include a mutation that inhibits complement fixation and Fc receptor binding, or it may be lytic or target-cell depleting, that is, able to destroy cells by binding complement or by another mechanism, such as antibody-dependent complement lysis. Accordingly, the Fc can be lytic and can activate complement and Fc receptor-mediated activities, leading to target cell lysis, allowing depletion of desired cells that express a TIM or TIM ligand.

The Fc region can be isolated from a naturally occurring source, recombinantly produced, or chemically synthesized using well known methods of peptide synthesis. For example, an Fc region that is homologous to the IgG C terminal domain can be produced by digestion of IgG with papain. IgG Fc has a molecular weight of approximately 50 kDa. The polypeptides of the invention can include the entire Fc region, or a smaller portion that retains the ability to lyse cells. In addition, full-length or fragmented Fc regions can be variants of the wild type molecule. That is, they can contain mutations that may or may not affect the function of the polypeptide. The Fc region can be derived from an IgG, such as human IgG1, IgG2, IgG3, IgG4, or analogous mammalian IgGs or from an IgM, such as human IgM or analogous mammalian IgMs. In a particular embodiment, the Fc region includes the hinge, CH2 and CH3 domains of human IgG1 or murine IgG2a.

The Fc region that can be part of the TIM targeting molecules or agents of the invention can be “target-cell depleting,” also referred to herein as lytic, or “non target-cell depleting,” also referred to herein as non-lytic. A non target-cell depleting Fc region typically lacks a high affinity Fc receptor binding site and a C′1q binding site. The high affinity Fc receptor binding site of murine IgG Fc includes the Leu residue at position 235 of IgG Fc. Thus, the murine Fc receptor binding site can be destroyed by mutating or deleting Leu 235. For example, substitution of Glu for Leu 235 inhibits the ability of the Fc region to bind the high affinity Fc receptor. The murine C′1q binding site can be functionally destroyed by mutating or deleting the Glu 318, Lys 320, and Lys 322 residues of IgG. For example, substitution of Ala residues for Glu 318, Lys 320, and Lys 322 renders IgG1 Fc unable to direct antibody-dependent complement lysis. In contrast, a target-cell depleting IgG Fc region has a high affinity Fc receptor binding site and a C′1q binding site and can reduce the amount of target cell, for example, by Fc lytic activity or other mechanisms, as disclosed herein. The high affinity Fc receptor binding site includes the Leu residue at position 235 of IgG Fc, and the C′1q binding site includes the Glu 318, Lys 320, and Lys 322 residues of IgG1. Target-cell depleting IgG Fc has wild type residues or conservative amino acid substitutions at these sites. Target-cell depleting IgG Fc can target cells for antibody dependent cellular cytotoxicity or complement directed cytolysis (CDC). Appropriate mutations for human IgG are also known (see, for example, Morrison et al., The Immunologist 2:119-124 (1994); and Brekke et al., The Immunologist 2:125, 1994). One skilled in the art can readily determine analogous residues for the Fc region of other species to generate target-cell depleting or non target-cell depleting fusions with a TIM targeting molecule or agent.

A variety of antigens can be used in a composition of the invention. Exemplary antigens include, but are not limited to, viral, bacterial, parasitic, and tumor associated antigens. The antigens can be in various forms, including but not limited to, whole inactivated organisms, protein antigens or peptide antigens derived therefrom, or other antigenic molecules suitable for eliciting an immune response against an organism or cell type. The antigen can also be in the form of a nucleic acid encoding an antigen, such as used in nucleic acid vaccines. As disclosed herein, a composition of the invention can be used to enhance an immune response in the presence of a TIM targeting molecule or agent relative to a composition lacking a TIM targeting molecule or agent (see Examples). An enhanced immune response was observed for hepatitis B virus, anthrax, influenza virus and HIV (see Examples VI-X). An enhanced immune response was also observed in a cancer model (see Example XII).

Exemplary antigens that can be used in composition of the invention include, but are not limited to, hepatitis B virus, influenza virus, anthrax, Listeria, Clostridium botulinum, tuberculosis, in particular multi-drug resistant strains, tularemia, Variola major (smallpox), viral hemorrhagic fevers, Yersinia pestis (plague), HIV, and other antigens associated with an infectious agent. Additional exemplary antigens include antigens associated with a tumor cell, antigens or antibodies against an antigen associated with an auto-immune disease, or antigens associated with allergy and asthma. Such an antigen can be included in a composition of the invention containing a TIM targeting molecule or agent for use as a vaccine against the respective disease.

In one embodiment, the methods and compositions of the invention can be used to treat an individual who has an infection or is at risk of having an infection by including an antigen from the infectious agent. An infection refers to a disease or condition attributable to the presence in a host of a foreign organism or agent that reproduces within the host. Infections typically involve breach of a mucosal or other tissue barrier by an infectious organism or agent. A subject that has an infection is a subject having objectively measurable infectious organisms or agents present in the subject's body. A subject at risk of having an infection is a subject that is predisposed to develop an infection. Such a subject can include, for example, a subject with a known or suspected exposure to an infectious organism or agent. A subject at risk of having an infection also can include a subject with a condition associated with impaired ability to mount an immune response to an infectious organism or agent, for example, a subject with a congenital or acquired immunodeficiency, a subject undergoing radiation therapy or chemotherapy, a subject with a burn injury, a subject with a traumatic injury, a subject undergoing surgery or other invasive medical or dental procedure, or a similarly immunocompromised individual.

Infections are broadly classified as bacterial, viral, fungal, or parasitic based on the category of infectious organism or agent involved. Other less common types of infection are also known in the art, including, for example, infections involving rickettsiae, mycoplasmas, and agents causing scrapie, bovine spongiform encephalopathy (BSE), and prion diseases (for example, kuru and Creutzfeldt-Jacob disease). Examples of bacteria, viruses, fungi, and parasites which cause infection are well known in the art. An infection can be acute, subacute, chronic, or latent, and it can be localized or systemic. Furthermore, an infection can be predominantly intracellular or extracellular during at least one phase of the infectious organism's or agent's life cycle in the host.

Bacteria include both Gram negative and Gram positive bacteria. Examples of Gram positive bacteria include, but are not limited to Pasteurella species, Staphylococci species, and Streptococcus species. Examples of Gram negative bacteria include, but are not limited to, Escherichia coli, Pseudomonas species, and Salmonella species. Specific examples of infectious bacteria include but are not limited to: Helicobacter pyloris, Borrelia burgdorferi, Legionella pneumophilia, Mycobacteria spp. (for example, M. tuberculosis, M. avium, M. intracellilare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, pathogenic Campylobacter spp., Enterococcus spp., Haemophilus influenzae, Bacillus anthracis, Corynebacterium diphtheriae, Corynebacterium spp., Erysipelothrix rhusiopathiae, Clostridium perfringens, Clostridium tetani, Enterobacter aerogenes, Klebsiella pneumoniae, Pasturella multocida, Bacteroides spp., Fusobacterium nucleatum, Streptobacillus moniliformis, Treponema pallidum, Treponema pertenue, Leptospira, Rickettsia, and Actinomyces israelii.

Examples of virus that have been found to cause infections in humans include but are not limited to: Retroviridae (for example, human immunodeficiency viruses, such as HIV-1 (also referred to as HTLV-III), HIV-2, LA V or IDLV-III/LA V, or HIV-III, and other isolates, such as HIV-LP; Picomaviridae (for example, polio viruses, hepatitis A virus; enteroviruses, human Coxsackie viruses, rhinoviruses, echoviruses); Calciviridae (for example, strains that cause gastroenteritis); Togaviridae (for example, equine encephalitis viruses, rubella viruses); Flaviviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (for example, reoviruses, orbiviurses and rotaviruses); Bimaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxviridae (variola viruses, vaccinia viruses, pox viruses); and Iridoviridae (for example, African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=enterally transmitted; class 2=parenterally transmitted (that is, Hepatitis C); Norwalk and related viruses, and astroviruses).

Examples of fungi include: Aspergillus spp., Blastomyces dermatitidis, Candida albicans, other Candida spp., Coccidioides immitis, Cryptococcus neoformans, Histoplasma capsulatum, Chiamydia trachomatis, Nocardia spp., Pneumocystis carinii.

Parasites include but are not limited to blood-borne and/or tissues parasites such as Babesia microti, Babesia divergens, Entamoeba histolytica, Giardia lamblia, Leishmania tropica, Leishmania spp., Leishmania braziliensis, Leishmania donovdni, Plasmodium falciparum, Plasmodium malariae, Plasmodium ovale, Plasmodium vivax, and Toxoplasma gondii, Trypanosoma gambiense and Trypanosoma rhodesiense (African sleeping sickness), Trypanosoma cruzi (Chagas' disease), and Toxoplasma gondii, flat worms, round worms.

The invention additionally provides methods of using a composition of the invention. In one embodiment, the invention provides a method of stimulating an immune response in an individual by administering a composition comprising an antigen and a TIM targeting molecule or agent in a pharmaceutically acceptable carrier. Such a TIM targeting molecule can be a TIM antibody such as an antibody to TIM-1, -2, -3, or -4.

As disclosed herein, the compositions of the invention can be used in methods of stimulating or enhancing an immune response to an antigen. The invention provides methods of stimulating an immune response by administering a composition of the invention containing a TIM targeting molecule or agent and an antigen. The inclusion of a TIM targeting molecule or agent can function as an adjuvant that enhances the immune response relative to a composition lacking the TIM targeting molecule or agent (see Examples).

The compositions and methods of the invention can be used to stimulate an immune response for preventing and/or treating a variety of diseases. Such diseases include infectious diseases including, but not limited to, diseases caused by viral, bacterial or parasitic organisms such as hepatitis B virus, influenza virus, anthrax, Listeria, Clostridium botulinum, tuberculosis, in particular multi-drug resistant strains, tularemia, Variola major (smallpox), viral hemorrhagic fevers, Yersinia pestis (plague), HIV, and other infectious agents, as disclosed herein.

The compositions and methods of the invention can additionally be used to treat a subject who has cancer or is at risk of having cancer. Cancer is a condition of uncontrolled growth of cells which interferes with the normal functioning of bodily organs and systems. A subject that has a cancer is a subject having objectively measurable cancer cells present in the subject's body. A subject at risk of having a cancer is a subject that is predisposed to develop a cancer. Such a subject can include, for example, a subject with a family history of or a genetic predisposition toward developing a cancer. A subject at risk of having a cancer also can include a subject with a known or suspected exposure to a cancer-causing agent.

Cancers which migrate from their original location and seed vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia), ultimately causing death.

A metastasis is a region of cancer cells, distinct from the primary tumor location, resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Compositions and methods of the invention can also be used to treat a variety of cancers or a subject at risk of developing a cancer by including a tumor associated antigen in the composition. As used herein, a “tumor associated antigen” is a tumor antigen that is expressed in a tumor cell. A number of tumor associated antigens are well known in the art to be associated with particular tumor cells and can be included in a composition of the invention to treat a variety of cancers, including but not limited to, breast, prostate, colon, and blood cancers, including leukemia, chronic lymphocytic leukemia (CLL), and the like. Methods of the invention can be used to stimulate an immune response to treat a tumor by inhibiting or slowing the growth of the tumor or decreasing the size of the tumor (see Example XII). A tumor associated antigen can also be a tumor specific antigen in that the antigen is expressed predominantly, although not necessarily exclusively, on a cancer cell. In such a case, it is understood that the tumor specific antigen can be advantageously targeted, allowing selective targeting to tumor cells.

Additional cancers include, but are not limited to, basal cell carcinoma, biliary tract cancer; bladder cancer; bone cancer; brain and central nervous system (CNS) cancer; cervical cancer; choriocarcinoma; colorectal cancers; connective tissue cancer; cancer of the digestive system; endometrial cancer; esophageal cancer; eye cancer; head and neck cancer; gastric cancer; intra-epithelial neoplasm; kidney cancer; larynx cancer; liver cancer; lung cancer (for example, small cell and non-small cell); lymphoma including Hodgkin's and non-Hodgkin's lymphoma; melanoma; myeloma; neuroblastoma; oral cavity cancer (for example, lip, tongue, mouth, and pharynx); ovarian cancer; pancreatic cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system, as well as other carcinomas and sarcomas.

Examples of cancer immunotherapies which are currently being used or which are in development include but are not limited to Rituxan™, IDEC-C2B8, anti-CD20 Mab, Panorex™, 3622W94, anti-EGP40 (17-1A), pancarcinoma antigen on adenocarcinomas, Herceptin™, anti-Her2, Anti-EGFr, BEC2, anti-idiotypic-GD3 epitope, Ovarex™, B43.13, anti-idiotypic CA125, 4B5, Anti-VEGF, RhuMAb, MDX-210, anti-HER-2, MDX-22, MDX-220, MDX-447, MDX-260, anti-GD-2, Quadramet™, CYT-424, IDEC-Y2B8, Oncolym™, Lym-1, SMART M195, ATRAGEN™, LDP-03, anti-CAMPATH, ior t6, anti CD6, MDX-11, OV1IO3, Zenapax™, Anti-Tac, anti-IL-2 receptor, MELIMMUNE-1 and -2, CEACIDE™, Pretarget™, NovoMAb-G2, TNT, anti-histone, Gliomab-H, GNI-250, EMD-72000, LymphoCide, CMA 676, Monopharm-C, ior egf/r3, ior c5, anti-FLK-2, SMART 1D1O, SMART ABL 364, and ImmuRAIT -CEA.

Cancer vaccines are medicaments used to stimulate an endogenous immune response against cancer cells. Currently produced vaccines predominantly activate the humoral immune system, that is, the antibody dependent immune response. Other vaccines currently in development are focused on activating the cell-mediated immune system, including cytotoxic T lymphocytes, which are capable of killing tumor cells. Cancer vaccines generally enhance the presentation of cancer antigens to both antigen presenting cells (APCs), for example, macrophages and dendritic cells, and/or to other immune cells such as T cells, B cells, and NK cells. Although cancer vaccines can take one of several forms, as discussed herein, their purpose is to deliver cancer antigens and/or cancer associated antigens to APCs in order to facilitate the endogenous processing of such antigens by APC and the ultimate presentation of antigen on the cell surface in the context of MHC class I molecules. One form of cancer vaccine is a whole cell vaccine, which is a preparation of cancer cells which have been removed from a subject, treated ex vivo, generally to kill the cancer cells or prevent them from proliferating, and then reintroduced as whole cells in the subject. Lysates of tumor cells can also be used as cancer vaccines to elicit an immune response. Another form of cancer vaccine is a peptide vaccine which uses cancer-specific or cancer-associated small proteins to activate T cells. Cancer-associated proteins are proteins which are not exclusively expressed by cancer cells, that is, other normal cells can still express these antigens. However, the expression of cancer-associated antigens is generally consistently up-regulated with cancers of a particular type. Yet another form of cancer vaccine is a dendritic cell vaccine, which includes whole dendritic cells which have been exposed to a cancer antigen or a cancer-associated antigen in vitro. Lysates or membrane fractions of dendritic cells can also be used as cancer vaccines. Dendritic cell vaccines are able to activate APCs directly. Other cancer vaccines include ganglioside vaccines, heat-shock protein vaccines, viral and bacterial vaccines, and nucleic acid vaccines.

The compositions and methods of the invention can additionally be used to treat autoimmune diseases, for example, multiple sclerosis, rheumatoid arthritis, type 1 diabetes, psoriasis or other autoimmune disorders. Autoimmune diseases are a class of diseases in which a subject's own antibodies react with host tissue or in which immune effector T cells are autoreactive to endogenous self-peptides and cause destruction of tissue. Thus, an immune response is mounted against a subject's own antigens, referred to as self-antigens. Autoimmune diseases include the examples described above and also Crohn's disease and other inflammatory bowel diseases such as ulcerative colitis, systemic lupus erythematosus (SLE), autoimmune encephalomyelitis, myasthenia gravis (MG), Hashimoto's thyroiditis, Goodpasture's syndrome, pemphigus (for example, pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (for example, crescentic glomerulonephritis, proliferative glomerulonephritis), bullous pemphigoid, Sjogren's syndrome, psoriatic arthritis, insulin resistance, autoimmune diabetes mellitus (type 1 diabetes mellitus; insulin-dependent diabetes mellitus), autoimmune hepatitis, autoimmune hemophilia, autoimmune lymphoproliferative syndrome (ALPS), autoimmune uveoretinitis, and Guillain-Barre syndrome. Recently, autoimmune disease has been recognized also to encompass atherosclerosis and Alzheimer's disease. A self-antigen refers to an antigen of a normal host tissue. Normal host tissue does not include cancer cells. Thus, an immune response mounted against a self-antigen, in the context of an autoimmune disease, is an undesirable immune response and contributes to destruction and damage of normal tissue, whereas an immune response mounted against a cancer antigen is a desirable immune response and contributes to destruction of the tumor or cancer.

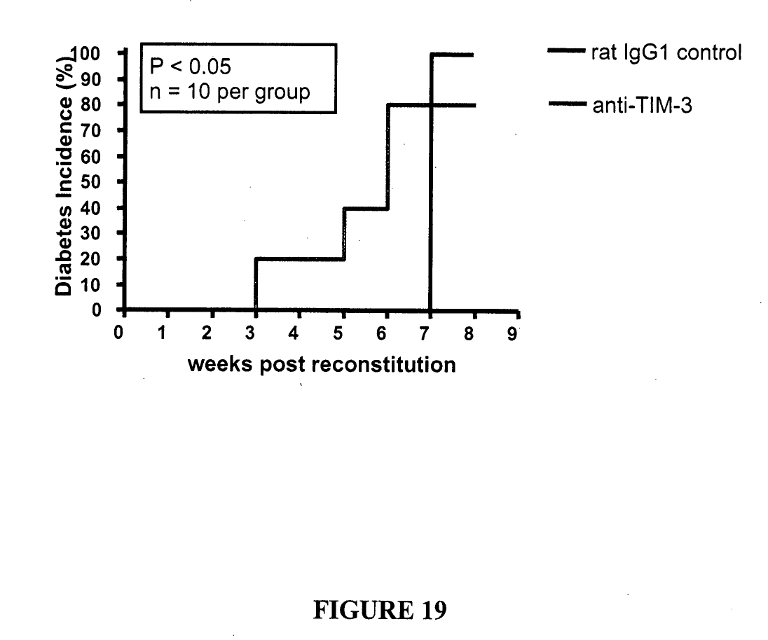

As exemplified in FIG. 19, TIM-3 signaling accelerates diabetes in mice (see Sanchez-Fueyo et al., Nat. Immunol. 4:1093-1101 (2003)). NOD-SCID mice received T cells from diabetic mice and were treated with control Ig or anti-TIM-3 (100 [ig twice a week for the duration of the experiment). Administration of anti-TIM-3 accelerated diabetes development, a Th1-mediated disease, demonstrating that TIM-3 functions in regulating Th1 function. Therefore, interference with one or more TIM-3 signaling pathways using a TIM-3 targeting molecules can be used to treat diabetes.

The compositions and methods of the invention can also be used to treat asthma and allergic reactions. Asthma is a disorder of the respiratory system characterized by inflammation and narrowing of the airways and increased reactivity of the airways to inhaled agents. Asthma is frequently, although not exclusively, associated with atopic or allergic symptoms. Allergy is an acquired hypersensitivity to a substance (allergen). Allergic conditions include eczema, allergic rhinitis or coryza, hay fever, bronchial asthma, urticaria (hives) and food allergies, and other atopic conditions. A “subject having an allergy” is a subject that has or is at risk of developing an allergic reaction in response to an allergen. An “allergen” refers to a substance that can induce an allergic or asthmatic response in a susceptible subject. There are numerous allergens, including pollens, insect venoms, animal dander, dust, fungal spores and drugs (for example, penicillin).

Examples of natural animal and plant allergens include proteins specific to the following genuses: Canine (Canisfamiliaris); Dermatophagoides (e.g., Dermatophagoides farinae); Felis (Felis domesticus); Ambrosia (Ambrosia artemuisfolia; Lotium (for example, Lotium perenne or Lotium multiflorum); Cryptomeria (Cryptomeriajaponica); Alternaria (Alternaria alternata); Alder; Alinus (Alnus gultinosa); Betula (Betula verrucosa); Quercus (Quercus alba); Olea (Olea europa); Artemisia (Artemisia vulgaris); Plantago (for example, Plantago lanceolata); Parietaria (for example, Parietaria officinalis or Parietaria judaica); Blattella (for example, Blattella gennanica); Apis (for example, Apis multiflornm); Cupressus (for example, Cupressus sempervirens, Cupressus arizonica and Cupressus macrocarpa); Juniperus (for example, Juniperus sabinoides, Juniperus virginiana, Juniperus communis and Juniperns ashei); Thuya (for example, Thuya orientalis); Chamaecyparis (for example, Chamaecyparis obtusa); Periplaneta (for example, Periplaneta americana); Agropyron (for example, Agropyron repens); Secale (for example, Secale cereale); Triticum (for example, Triticum aestivum); Dactylis (for example, Dactylis glomerata); Festuca (for example, Festuca elatior); Poa (for example, Poa pratensis or Poa compressa); Avena (for example, Avena sativa); Holcus (for example, Holcus lanatus); Anthoxanthum (for example, Anthoxanthum odoratum); Arrhenatherum (for example, Arrhenatherum elatius); Agrostis (for example, Agrostis alba); Phleum (for example, Phleum pratense); Phalaris (e.g., Phalaris arundinacea); Paspalum (for example, Paspalum notatum); Sorghum (for example, Sorghum halepensis); and Bromus (for example, Bromus inermis).

Furthermore, the compositions and methods of the invention can be used for transplantation to inhibit organ rejection and in heart disease by affecting inflammatory cytokines. Effects of various TIM targeting molecules in various disease models are illustrated in FIG. 19 and in Examples VI-XII. Treatment with TIMs or anti-TIM antibodies promoted a stronger immune response induced by vaccination.

The methods of the invention can be used to increase Th1 or Th2 as advantageous for a particular indication. For example, Th1 cytokines are appropriate for intracellular pathogens such as bacteria or viruses, cancer and delayed-type hypersensitivity. Th2 cytokines are appropriate for extracellular helminthic parasites such as tapeworms and nematodes and for the development of antibody responses to neutralize circulating viruses and bacteria. In contrast, inappropriate Th1 responses result in autoimmune disorders, for example, multiple sclerosis, psoriasis, rheumatoid arthritis, and type 1 diabetes, and transplant rejection; lack of Th1 cytokines results in the inability to fight intracellular pathogens such as viruses and bacteria. Inappropriate Th2 responses result in asthma, allergic disorders, inability to clear intracellular infections, and susceptibility to HIV; lack of Th2 cytokines results in the inability to neutralize invading viruses and bacteria.

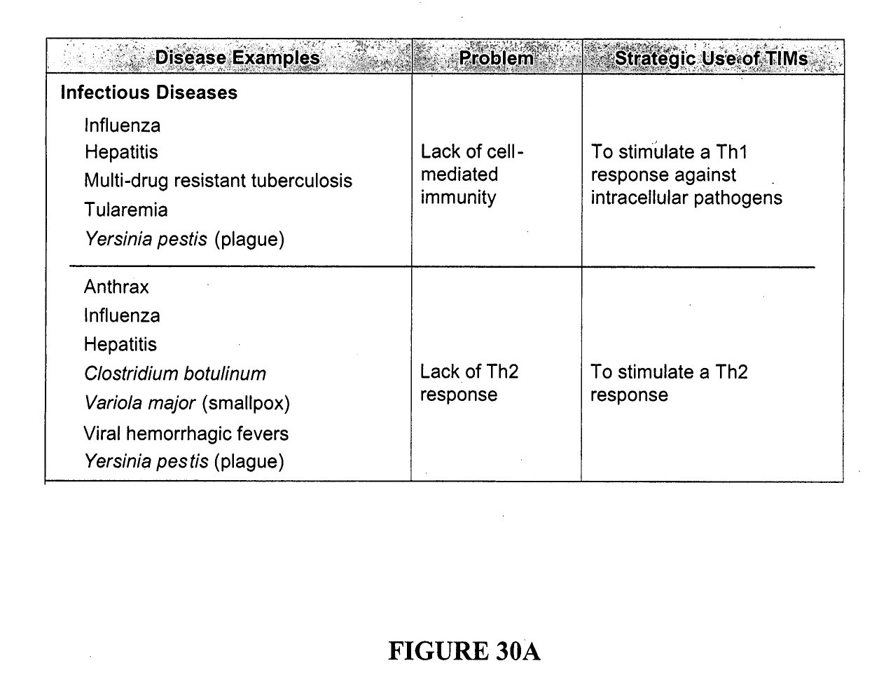

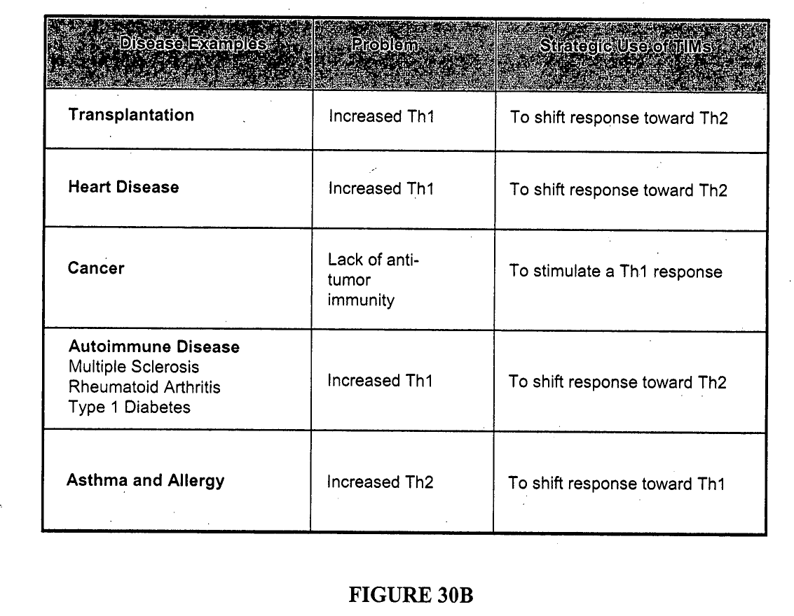

The methods of the invention are advantageous because they can be used to increase a Th1 or Th2 response, as desired. As the immune response progresses, TIM molecules are expressed and help direct the secretion of appropriate cytokine messengers. TIM-1 functions in stimulating Th2, whereas TIM-3 functions in stimulating Th1. Thus, the use of a particular TIM targeting molecule can be used to modulate the relative amount of Th1 or Th2, as useful for a particular desired immune response. Exemplary diseases and how a desired effect of a TIM targeting molecules can be used to enhance an immune response for treatment of various diseases are described in FIG. 30.

It is understood that the compositions and methods of the invention can be combined with other therapies for treating a particular condition. For example, the use of a composition of the invention as a cancer vaccine can be optionally used in combination with other cancer therapies such as well known chemotherapies or radiotherapies. Similarly, the use of a composition of the invention for treating autoimmune diseases can be optionally combined with therapies used to treat a particular autoimmune disease. Likewise, a composition of the invention for treating asthma or an allergic condition can optionally be combined with therapies for the respective conditions.

The compositions and methods of the invention can be used for therapeutic and/or diagnostic purposes, which can be for human or veterinary applications. For example, the compositions of the invention can be used to target a therapeutic or diagnostic moiety. In the case of a therapeutic moiety, the moiety can be a drug such as a chemotherapeutic agent, cytotoxic agent, toxin, and the like. For example, a cytotoxic agent can be a radionuclide or chemical compound. Exemplary radionuclides useful as therapeutic agents include, for example, X-ray or γ-ray emitters. In addition, a moiety can be a drug delivery vehicle such as a chambered microdevice, a cell, a liposome or a virus, which can contain an agent such as a drug or a nucleic acid.

Exemplary therapeutic agents include, for example, the anthracyclin doxorubicin, which has been linked to antibodies and the antibody/doxorubicin conjugates have been therapeutically effective in treating tumors (Sivam et al., Cancer Res. 55:2352-2356 (1995); Lau et al., Bioorg. Med. Chem. 3:1299-1304 (1995); Shih et al., Cancer Immunol. Immunother. 38:92-98 (1994)). Similarly, other anthracyclins, including idarubicin and daunorubicin, have been chemically conjugated to antibodies, which have delivered effective doses of the agents to tumors (Rowland et al., Cancer Immunol. Immunother. 37:195-202 (1993); Aboud-Pirak et al., Biochem. Pharmacol. 38:641-648 (1989)).

In addition to the anthracyclins, alkylating agents such as melphalan and chlorambucil have been linked to antibodies to produce therapeutically effective conjugates (Rowland et al., Cancer Immunol. Immunother. 37:195-202 (1993); Smyth et al., Immunol. Cell Biol. 65:315-321 (1987)), as have vinca alkaloids such as vindesine and vinblastine (Aboud-Pirak et al., supra, 1989; Starling et al., Bioconi. Chem. 3:315-322 (1992)). Similarly, conjugates of antibodies and antimetabolites such as 5-fluorouracil, 5-fluorouridine and derivatives thereof have been effective in treating tumors (Krauer et al., Cancer Res. 52:132-137 (1992); Henn et al., J. Med. Chem. 36:1570-1579 (1993)). Other chemotherapeutic agents, including cis-platinum (Schechter et al., Int. J. Cancer 48:167-172 (1991)), methotrexate (Shawler et al., J. Biol. Resp. Mod. 7:608-618 (1988); Fitzpatrick and Garnett, Anticancer Drug Des. 10:11-24 (1995)) and mitomycin-C (Dillman et al., Mol. Biother. 1:250-255 (1989)) also are therapeutically effective when administered as conjugates with various different antibodies. A therapeutic agent can also be a toxin such as ricin.

A therapeutic agent can also be a physical, chemical or biological material such as a liposome, microcapsule, micropump or other chambered microdevice, which can be used, for example, as a drug delivery system. Generally, such microdevice, should be nontoxic and, if desired, biodegradable. Various moieties, including microcapsules, which can contain an agent, and methods for linking a moiety, including a chambered microdevice, to a TIM targeting molecule or agent of the invention are well known in the art and commercially available (see, for example, “Remington's Pharmaceutical Sciences” 18th ed. (Mack Publishing Co. 1990), chapters 89-91; Harlow and Lane, Antibodies: A laboratory manual (Cold Spring Harbor Laboratory Press 1988; Hermanson, Bioconjugate Techniques, Academic Press, San Diego (1996)).

For diagnostic purposes, a TIM targeting molecule or agent can further comprise a detectable moiety. A detectable moiety can be, for example, a radionuclide, fluorescent, magnetic, colorimetric moiety, and the like. For in vivo diagnostic purposes, a moiety such as a gamma ray emitting radionuclide, for example, indium-111 or technitium-99, can be linked to an antibody of the invention and, following administration to a subject, can be detected using a solid scintillation detector. Similarly, a positron emitting radionuclide such as carbon-11 or a paramagnetic spin label such as carbon-13 can be linked to the molecule and, following administration to a subject, the localization of the moiety can be detected using positron emission transaxial tomography or magnetic resonance imaging, respectively. Such methods can identify a primary tumor as well as a metastatic lesion.

For diagnostic purposes, the TIM targeting molecule or agent can be used for in vivo diagnosis or in vitro in a tissue sample obtained from an individual, for example, by tissue biopsy. Exemplary bodily fluids include, but are not limited to, serum, plasma, urine, synovial fluid, and the like.

A therapeutic or detectable moiety can be coupled to a TIM targeting molecule or agent by any of a number of well known methods for coupling or conjugating moieties. It is understood that such coupling methods allow the attachment of a therapeutic or detectable moiety without interfering or inhibiting the binding activity of the TIM targeting molecule or agent. Methods for conjugating moieties to a TIM targeting molecule or agent of the invention are well known to those skilled in the art (see, for example, Hermanson, Bioconjugate Techniques, Academic Press, San Diego (1996)). It is further understood that a therapeutic or detectable moiety can be non-covalently conjugated to a TIM targeting molecule or agent so long as the non-covalently bound conjugate has sufficient binding affinity for a desired purpose. For example, the therapeutic or detectable moiety can be conjugated to a TIM targeting molecule by conjugating biotin or avidin to the respective moiety and TIM targeting molecule and using biotin-avidin to non-covalently conjugate the moiety and TIM targeting molecule. Other types of well known binding molecule pairs can similarly be used including, for example, maltose binding protein/maltose, glutathione-S transferase/glutathione, and the like.

Thus, in an embodiment of the invention, a TIM targeting molecule or agent, for example, an anti-TIM antibody or TIM protein, can be used as a delivery system for the specific targeting of toxic radioactive isotopes or toxins to cancer cells or to autoreactive B and T cells expressing the appropriate TIM molecule (targeted by an anti-TIM antibody) or TIM ligand molecule (targeted by a TIM protein) on the cell surface. Antibodies or recombinant proteins, such as TIM proteins, for example, TIM proteins with a Fc tail, can be conjugated to plant toxins like Ricin, abrin, pokeweed antiviral protein, saporin, gelonin and the like or bacterial toxin like Pseudomonas exotoxin, diphtheria toxin, or chemical toxin such as calicheamicin and esperamicin, duocarmycin, doxorubicin, melphalan, methotrexate, chlorambucil, cytarabine or cytosine arabinoside (ARA-C), vindesine, cis-platinum, etoposide, bleomycin, mitomycin C and 5-fluorouracil; or radioisotopes like Iodine-131 or Yttrium-90.

In one embodiment, a composition of the invention can be conjugated covalently or non-covalently to toxic molecules including chemical, bacterial or plant toxins and radioactive isotopes. In another embodiment, the invention provides a method for treatment of cancer or autoimmune diseases wherein the TIM targeting molecule or agent, for example, an anti-TIM antibody or TIM protein, is conjugated covalently or non-covalently to a therapeutic moiety such as a toxic molecule, including chemical, bacterial or plant toxins and radioactive isotopes for use as a therapeutic modality. Combinations of the various toxins could also be coupled to one antibody molecule. Other chemotherapeutic agents are known to those skilled in the art, as disclosed herein.

In an additional embodiment, the invention provides the use of a composition comprising a TIM targeting molecule or agent conjugated to a therapeutic moiety such as an immunotoxin for the manufacture of a medicament for treating an autoimmune disorder in a subject. In yet a further embodiment, the invention provides the use of a TIM targeting molecule or agent conjugated to a therapeutic moiety where the autoimmune disorder is a disorder selected from rheumatoid arthritis, multiple sclerosis, autoimmune diabetes mellitus, systemic lupus erythematosus, autoimmune lymphoproliferative syndrome (ALPS), and the like.

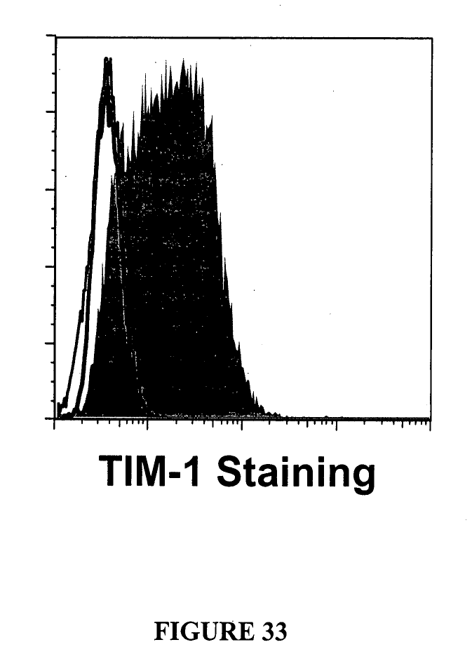

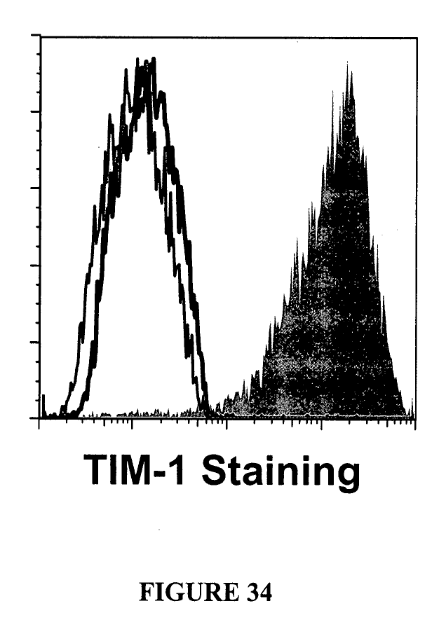

In still another embodiment, the invention provides the use of a TIM targeting molecule or agent for the treatment of cancer in a subject. For example, the cancer can be a carcinoma, sarcoma or lymphoma, or other cancer types. A TIM targeting molecule or agent can be used for the treatment of tumors that express the appropriate TIM or TIM ligand. A TIM or TIM ligand can be identified in tumor biopsy samples. As disclosed herein, various cell lines have been shown to express TIM or TIM ligands, including renal adenocarcinoma, thymomas and lymphomas (see Example XV and FIGS. 33-36). If a tumor biopsy sample is positive for TIM expression, then a TIM targeting molecule such as an anti-TIM antibody conjugated with a cytotoxic agent can be used to target tumor cells. On the other hand, if the tumor expresses an appropriate ligand for TIM molecules, then the appropriate TIM molecule by itself or as a fusion protein conjugated to a cytotoxic agent can be used for targeting the TIM ligand-expressing tumor. Similarly, a TIM targeting molecule or agent, or a conjugate thereof with a therapeutic or diagnostic moiety, can be used to target various cell types or tissues that express a TIM or TIM ligand.

The invention provides a composition comprising a TIM targeting molecule conjugated to a therapeutic or diagnostic moiety. The therapeutic moiety can be a chemotherapeutic agent, cytotoxic agent or toxin. The cytotoxic agent can be, for example, a radionuclide or chemical compound, including but not limited to the chemical compound calicheamicin, esperamicin, duocarmycin, doxorubicin, melphalan, methotrexate, chlorambucil, cytarabine, vindesine, cis-platinum, etoposide, bleomycin, mitomycin C and 5-fluorouracil or the radionuclide Iodine-131 or Yttrium-90. In a particular embodiment, the toxin can be a plant or bacterial toxin, including but not limited to the plant toxin ricin, abrin, pokeweed antiviral protein, saporin or gelonin or the bacterial from Pseudomonas exotoxin or diphtheria toxin.

Methods of making and administering compositions as vaccines are well known to those skilled in the art. The immunologically effective amounts of the components are determined empirically, but can be based, for example, on immunologically effective amounts in animal models. Factors to be considered include the antigenicity, the formulation, the route of administration, the number of immunizing doses to be administered, the physical condition, weight and age of the individual, and the like. Such factors are well known in the art and can be readily determined by those skilled in the art (see, for example, Paoletti and McInnes, eds., Vaccines, from Concept to Clinic: A Guide to the Development and Clinical Testing of Vaccines for Human Use CRC Press (1999). As disclosed herein, the TIM targeting molecules or agents can be used as an adjuvant (see Examples). It is understood that the TIM targeting molecules or agents of the invention can be used as an adjuvant alone or, if desired, in combination with other well known adjuvants.

Compositions of the invention can be administered locally or systemically by any method known in the art, including, but not limited to, intramuscular, intradermal, intravenous, subcutaneous, intraperitoneal, intranasal, oral or other mucosal routes. Additional routes include intracranial (for example, intracisternal or intraventricular), intraorbital, opthalmic, intracapsular, intraspinal, and topical administration. The compositions of the invention can be administered in a suitable, nontoxic pharmaceutical carrier, or can be formulated in microcapsules or as a sustained release implant. The immunogenic compositions of the invention can be administered multiple times, if desired, in order to sustain the desired immune response. The appropriate route, formulation and immunization schedule can be determined by those skilled in the art.

In a method of the invention, a composition of the invention can be administered so that the antigen and TIM targeting molecule are in a single composition that is administered so that the antigen and TIM targeting molecule are co-administered. Alternatively, a method of the invention can be performed so that the antigen and TIM targeting molecule are administered as separate compositions, for example, separate pharmaceutical compositions. Such separate compositions containing an antigen and TIM targeting molecule can be administered simultaneously, either by mixing the compositions together or injecting them at the same site, or the compositions can be administered separately at the same or a different location. The TIM targeting molecule can be administered at the same site as the antigen or a different site, and can be administered at the same time or sequentially over a period of a few minutes or a few days. One skilled in the art can readily determine a desired regimen for administration of the antigen and TIM targeting molecule for a desired effect. In the case where an antigen is already present, for example, with an ongoing infection or disease in which a disease-associated antigen is being exposed to the immune system, a TIM targeting molecule can be administered to stimulate an immune response against an antigen already being expressed in an individual.

A TIM targeting molecule can be administered in one or more different forms. If the TIM targeting molecule is a peptide or polypeptide, such as an anti-TIM antibody or a TIM fusion protein, modes of administration include, but are not limited to, administration of the purified peptide or polypeptide, administration of cells expressing the peptide or polypeptide, or administration of nucleic acids encoding the peptide or polypeptide.

The methods of the present invention and the therapeutic compositions used to carry them out contain “substantially pure” agents. For example, in the event the TIM targeting molecule or agent is a polypeptide, the polypeptide can be at least about 60% pure relative to other polypeptides or undesirable components in the original source of the polypeptide. For example, if a polypeptide is purified from a natural source, from recombinant expression, or chemical synthesis, the purity is relative to other components in the original natural source, recombinant source, or synthetic reaction. One skilled in the art can readily determine appropriate well known purification methods for a polypeptide agent or other agents of the invention. In particular, the agent can be at least about 75%, at least about 80%, at least about 85%, at least about 90%, at least about 95%, at least about 98% or at least about 99% purity. One skilled in the art can readily determine a suitable purity for a particular desired application. Purity can be measured by any appropriate standard method, for example, column chromatography, polyacrylamide gel electrophoresis, HPLC analysis, and can be based on desired quantification criteria such as ultraviolet absorbance, staining, or similar methods of measuring quantities depending on the chemical nature of the agent. It is understood that when an agent of the invention is combined with other components as an adjuvant, for example, in a vaccine, that the TIM targeting molecule or agent can be administered at a particular purity, for example 95% purity, but is not required to be 95% of the components in the vaccine such as antigen, buffer, and the like. One skilled in the art can readily determine a suitable purity and a suitable amount of the TIM targeting molecule or agent relative to other desirable components in a composition of the invention.

Although agents useful in the methods of the present invention can be obtained from naturally occurring sources, they can also be synthesized or otherwise manufactured, for example, by expression of a recombinant nucleic acid molecule encoding a TIM targeting molecule or agent. Methods for recombinantly expressing polypeptides are well known to those skilled in the art (Ausubel et al., Current Protocols in Molecular Biology (Supplement 56), John Wiley & Sons, New York (2001); Sambrook and Russel, Molecular Cloning: A Laboratory Manual, 3rd ed., Cold Spring Harbor Press, Cold Spring Harbor (2001)). Methods of peptide synthesis are also well known to those skilled in the art (Merrifield, J. Am. Chem. Soc. 85:2149 (1964); Bodanszky, Principles of Peptide Synthesis Springer-Verlag (1 984)). Polypeptides that are purified from a natural source, for example, from eukaryotic organisms, can be purified to be substantially free from their naturally associated components. Similarly, polypeptides that are expressed recombinantly in eukaryotic or prokaryotic cells, for example, E. coli or other prokaryotes, or that are chemically synthesized can be purified to a desired level of purity. In the event the polypeptide is a chimera, it can be encoded by a hybrid nucleic acid molecule containing one sequence that encodes all or part of the agent, for example, a sequence encoding a TIM polypeptide and sequence encoding an Fc region of IgG.

Agents of the invention, in particular, polypeptides expressed recombinantly, can be fused to an affinity tag to facilitate purification of the polypeptide. In one embodiment, the affinity tag can be a relatively small molecule that does not interfere with the function of the polypeptide, for example, binding of a TIM targeting molecule or agent. Alternatively, the affinity tag can be fused to a polypeptide with a protease cleavage site that allows the affinity tag to be removed from the recombinantly expressed polypeptide. The inclusion of a protease cleavage site is particularly useful if the affinity tag is relatively large and could potentially interfere with a function of the polypeptide. Exemplary affinity tags include a poly-histidine tag, generally containing about 5 to about 10 histidines, or hemagglutinin tag, which can be used to facilitate purification of recombinantly expressed polypeptides from prokaryotic or eukaryotic cells. Other exemplary affinity tags include maltose binding protein or lectins, both of which bind sugars, glutathione-S transferase, avidin, and the like. Other suitable affinity tags include an epitope for which a specific antibody is available. An epitope can be, for example, a short peptide of about 3-5 amino acids or more, a carbohydrate, a small organic molecule, and the like. Epitope tags have been used to affinity purify recombinant proteins and are commercially available. For example, antibodies to epitope tags, including myc, FLAG, hemagglutinin (HA), green fluorescent protein (GFP), polyHis, and the like, are commercially available (see, for example, Sigma, St. Louis Mo.; PerkinElmer Life Sciences, Boston Mass.).

In therapeutic applications, agents of the invention can be administered with a physiologically acceptable carrier, such as physiological saline. The therapeutic compositions of the invention can also contain a carrier or excipient, many of which are known to one of ordinary skill in the art. Excipients that can be used include buffers, for example, citrate buffer, phosphate buffer, acetate buffer, and bicarbonate buffer; amino acids; urea; alcohols; ascorbic acid; phospholipids; proteins, for example, serum albumin; ethylenediamine tetraacetic acid (EDTA); sodium chloride or other salts; liposomes; mannitol, sorbitol, glycerol, and the like. The agents of the invention can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences,” 18th ed., Mack Publishing Company, Easton Pa. (1990).

As discussed above, polypeptide agents of the invention, including those that are fusion proteins, can be obtained by expression of one or more nucleic acid molecules in a suitable eukaryotic or prokaryotic expression system and subsequent purification of the polypeptide agents. In addition, a polypeptide agent of the invention can also be administered to a patient by way of a suitable therapeutic expression vector encoding one or more nucleic acid molecules, either in vivo or ex vivo. Furthermore, a nucleic acid can be introduced into a cell of a graft prior to transplantation of the graft. Thus, nucleic acid molecules encoding the agents described above are within the scope of the invention.

Just as polypeptides of the invention can be described in terms of their identity with wild type polypeptides, the nucleic acid molecules encoding them will have a certain identity with those that encode the corresponding wild type polypeptides. For example, the nucleic acid molecule encoding TIM-1, TIM-2, TIM-3 or TIM-4 can be at least about 50%, at least about 65%, at least about 75%, at least 85%, at least about 90%, at least about 95%, at least about 98%, or at least about 99% identical to the nucleic acid encoding natural or wild-type TIM-1, TIM-2, TIM-3 or TIM-4. Similarly, the TIM polypeptides can have at least about 50%, at least about 65%, at least about 75%, at least 85%, at least about 90%, at least about 95%, at least about 98%, or at least about 99% identical to the natural or wild-type TIM-1, TIM-2, TIM-3 or TIM-4 polypeptides. It is understood that a polypeptide or encoding nucleic acid that has less than 100% identity with a corresponding wild type molecule still retains a desired function of the TIM polypeptide.

The nucleic acid molecules that encode agents of the invention can contain naturally occurring sequences, or sequences that differ from those that occur naturally, but, due to the degeneracy of the genetic code, encode the same polypeptide. These nucleic acid molecules can consist of RNA or DNA, for example, genomic DNA, cDNA, or synthetic DNA, such as that produced by phosphoramidite-based synthesis, or combinations or modifications of the nucleotides within these types of nucleic acids. In addition, the nucleic acid molecules can be double stranded or single stranded, either a sense or an antisense strand. It is understood by those skilled in the art that a suitable form of nucleic acid can be selected based on the desired use, for example, expression using viral vectors that are single or double stranded and are sense or antisense.

In the case of a naturally occurring nucleic acid molecule of the invention, the nucleic acid molecule can be “isolated” from the naturally occurring genome of an organism because they are separated from either the 5′ or the 3′ coding sequence with which they are immediately contiguous in the genome. Thus, a nucleic acid molecule includes a sequence that encodes a polypeptide and can include non-coding sequences that lie upstream or downstream from a coding sequence. Those of ordinary skill in the art are familiar with routine procedures for isolating nucleic acid molecules (see, for example, Sambrook et al., Molecular Cloning: A Laboratory Manual, 2nd ed., Cold Spring Harbor Press, Plainview, N.Y. (1989); Ausubel et al., Current Protocols in Molecular Biology (Supplement 56), John Wiley & Sons, New York (2001); and Sambrook and Russel, Molecular Cloning: A Laboratory Manual, 3rd ed., Cold Spring Harbor Press, Cold Spring Harbor (2001)). The nucleic acid can, for example, be generated by treatment of genomic DNA with restriction endonucleases, or by performance of the polymerase chain reaction (PCR) to amplify a desired region of genomic DNA or cDNA using well known methods (see, for example, Dieffenbach and Dveksler, PCR Primer: A Laboratory Manual, Cold Spring Harbor Press (1995)). In the event the nucleic acid molecule is a ribonucleic acid (RNA), molecules can be produced by in vitro transcription.

The isolated nucleic acid molecules of the invention can include fragments not found in the natural state. Thus, the invention encompasses recombinant molecules, such as those in which a nucleic acid sequence, for example, a sequence encoding TIM-1, TIM-2 TIM-3 or TIM-4, is incorporated into a vector, for example, a plasmid or viral vector, or into the genome of a heterologous cell or the genome of a homologous cell, at a position other than the natural chromosomal location.

As described above, agents of the invention can be fusion proteins. In addition to, or in place of, the heterologous polypeptides described above, a nucleic acid molecule encoding an agent of the invention can contain sequences encoding a “marker” or “reporter.” Examples of marker or reporter genes include β lactamase, chloramphenicol acetyltransferase (CAT), adenosine deaminase (ADA), aminoglycoside phosphotransferase (neo^{r}, G418^{r}), dihydrofolate reductase (DHFR), hygromycin B-phosphotransferase (HPH), thymidine kinase (TK), lacZ (encoding β galactosidase), and xanthine guanine phosphoribosyltransferase (XGPRT). As with many of the standard procedures associated with the practice of the invention, one of ordinary skill in the art will be aware of additional useful reagents, for example, of additional sequences that can serve the function of a marker or reporter.

The nucleic acid molecules of the invention can be obtained by introducing a mutation into an agent of the invention, for example, a TIM-1, TIM-2, TIM-3 or TIM-4 molecule, obtained from any biological cell, such as the cell of a mammal, or produced by routine cloning methods. Thus, the nucleic acids of the invention can be those of a mouse, rat, guinea pig, cow, sheep, horse, pig, rabbit, monkey, baboon, dog, or cat. In a particular embodiment, the nucleic acid molecules can encode a human TIM.

A nucleic acid molecule of the invention described herein can be contained within a vector that is capable of directing its expression in, for example, a cell that has been transduced with the vector. Accordingly, in addition to polypeptide agents, expression vectors containing a nucleic acid molecule encoding those agents and cells transfected with those vectors are provided.

Vectors suitable for use in the present invention include T7 based vectors for use in bacteria (see, for example, Rosenberg et al., Gene 56:125-135 (1987), the pMSXND expression vector for use in mammalian cells (Lee and Nathans, J. Biol. Chem. 263:3521-3527 (1988), yeast expression systems, such as Pichia pastoris, for example the PICZ family of expression vectors (Invitrogen, Carlsbad, Calif.) and baculovirus derived vectors, for example the expression vector pBacPAK9 (Clontech, Palo Alto, Calif.) for use in insect cells. The nucleic acid inserts, which encode the polypeptide of interest in such vectors, can be operably linked to a promoter, which is selected based on, for example, the cell type in which the nucleic acid is to be expressed. For example, a T7 promoter can be used in bacteria, a polyhedrin promoter can be used in insect cells, and a cytomegalovirus or metallothionein promoter can be used in mammalian cells. Also, in the case of higher eukaryotes, tissue specific and cell type specific promoters are widely available. These promoters are so named for their ability to direct expression of a nucleic acid molecule in a given tissue or cell type within the body. One of ordinary skill in the art can readily determine a suitable promoter and/or other regulatory elements that can be used to direct expression of nucleic acids in a desired cell or organism.

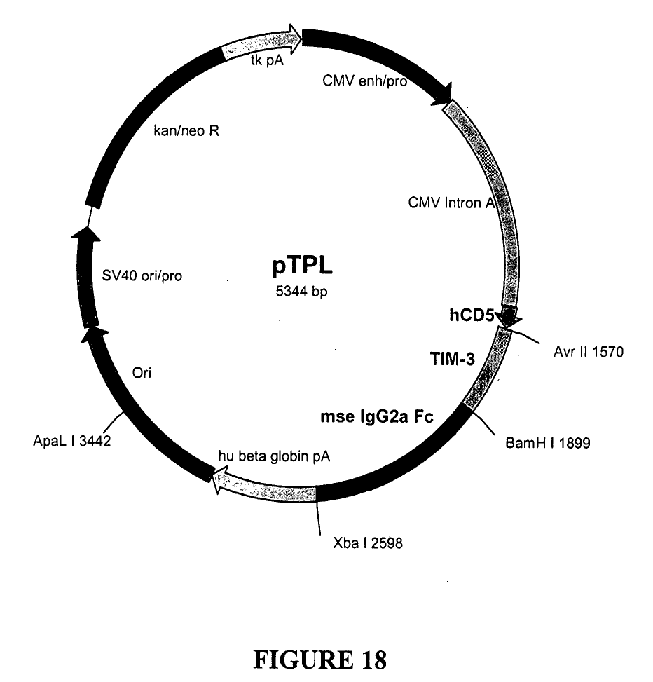

In addition to sequences that facilitate transcription of the inserted nucleic acid molecule, vectors can contain origins of replication, and other genes that encode a selectable marker. For example, the neomycin-resistance (neo^{r}) gene imparts G418 resistance to cells in which it is expressed, and thus permits phenotypic selection of the transfected cells. Other feasible selectable marker genes allowing for phenotypic selection of cells include various fluorescent proteins, for example, green fluorescent protein (GFP) and variants thereof. Those of skill in the art can readily determine whether a given regulatory element or selectable marker is suitable for a particular use. An exemplary vector is shown in FIG. 18.

Viral vectors that can be used in the invention include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain a nucleic acid molecule that encodes an agent of the invention and that express the protein encoded in the nucleic acid molecule are also provided. A cell of the invention is a transfected cell, that is, a cell into which one or more nucleic acid molecules, for example a nucleic acid molecule encoding a TIM-1, TIM-2, TIM-3 or TIM-4 polypeptide, or for example nucleic acids encoding for the heavy and light chains of an anti-TIM antibody, has been introduced by means of recombinant DNA techniques. The progeny of such a cell are also considered within the scope of the invention. A variety of expression systems can be utilized. For example, a TIM-1, TIM-2, TIM-3 or TIM-4 or anti-TIM polypeptide can be produced in a prokaryotic host, such as the bacterium E. coli, or in a eukaryotic host, such as an insect cell, for example, Sf21 cells, or mammalian cells, for example, COS cells, CHO cells, 293 cells, PER.C6 cells, NIH 3T3 cells, HeLa cells, and the like. These cells are available from many sources, including the American Type Culture Collection (Manassas, Va.). One skilled in the art can readily select appropriate components for a particular expression system, including expression vector, promoters, selectable markers, and the like, as discussed above, suitable for a desired cell or organism. The selection of use of various expression systems can be found, for example, in Ausubel et al., Current Protocols in Molecular Biology, John Wiley and Sons, New York, N.Y. (1993); and Pouwels et al., Cloning Vectors: A Laboratory Manual, 1985 Suppl. 1987). Also provided are eukaryotic cells that contain a nucleic acid molecule encoding an agent of the invention and express the protein encoded by such a nucleic acid molecule.

Furthermore, eukaryotic cells of the invention can be cells that are part of a cellular transplant, a tissue or organ transplant. Such transplants can comprise either primary cells taken from a donor organism or cells that were cultured, modified and/or selected in vitro before transplantation to a recipient organism, for example, eurkaryotic cells lines, including stem cells or progenitor cells. If, after transplantation into a recipient organism, cellular proliferation occurs, the progeny of such a cell are also considered within the scope of the invention. A cell, being part of a cellular, tissue or organ transplant, can be transfected with a nucleic acid encoding a TIM or anti-TIM polypeptide and subsequently be transplanted into the recipient organism, where expression of the polypeptide occurs. Furthermore, such a cell can contain one or more additional nucleic acid constructs allowing for application of selection procedures, for example, of specific cell lineages or cell types prior to transplantation into a recipient organism. Such transplanted cells can be used in therapeutic applications. For example, if the TIM targeting molecule or agent is a polypeptide, cells expressing the TIM targeting molecule can be transplanted to provide a source of the TIM targeting molecule using well known methods of gene delivery and suitable vectors (see, for example, Kaplitt and Loewy, Viral Vectors: Gene Therapy and Neuroscience Applications Academic Press, San Diego (1995)).

In the case of cell transplants, the cells can be administered either by an implantation procedure or with a catheter-mediated injection procedure through the blood vessel wall. In some cases, the cells may be administered by release into the vasculature, from which the cells subsequently are distributed by the blood stream and/or migrate into the surrounding tissue.

In another embodiment, a TIM targeting molecule that functions as an immunosuppressive agent can be introduced by gene delivery methods to cells of the organ. In such a case, the donor organ itself provides an immunosuppressive agent to facilitate organ transplant and inhibit transplant rejection.

The invention additionally provides a kit containing a composition comprising an antigen and a TIM targeting molecule or agent. The invention further provides a kit containing a composition comprising an antigen and a composition comprising a TIM targeting molecule or agent. As discussed above in regard to administering a composition of the invention, a kit containing separate compositions of antigen and TIM targeting molecule can be co-administered or can be administered separately, either in the same location or different locations. A kit containing separate antigen and TIM targeting molecule compositions can be administered contemporaneously or at different times, as disclosed herein.

As used herein, the term “antibody” is used in its broadest sense to include polyclonal and monoclonal antibodies, as well as antigen binding fragments of such antibodies. An antibody specific for an antigen, or an antigen binding fragment of such an antibody, is characterized by having specific binding activity for an antigen or an epitope thereof of at least about 1×10⁵M⁻¹. Thus, Fab, F(ab′)₂, Fd and Fv fragments of an antibody specific for an antigen, which retain specific binding activity for an antigen, are included within the definition of an antibody. Specific binding activity to an antigen such as a TIM can be readily determined by one skilled in the art, for example, by comparing the binding activity of an antibody to its respective antigen versus a non-antigen control molecule. One skilled in the art will readily understand the meaning of an antibody having specific binding activity for a particular antigen, for example, a TIM. The antibody can be a polyclonal or a monoclonal antibody. Methods of preparing polyclonal or monoclonal antibodies are well known to those skilled in the art (see, for example, Harlow and Lane, Antibodies: A Laboratory Manual, Cold Spring Harbor Laboratory Press (1988)). When using polyclonal antibodies, the polyclonal sera can be affinity purified using the antigen to generate mono-specific antibodies having reduced background binding and a higher proportion of antigen-specific antibodies.

In addition, the term “antibody” as used herein includes naturally occurring antibodies as well as non-naturally occurring antibodies, including, for example, single chain antibodies, chimeric, bifunctional and humanized antibodies, as well as antigen-binding fragments thereof. Humanized antibodies are meant to include recombinant antibodies generated by combining human immunoglobulin sequences, for example, human framework sequences, with non-human immunoglobulin sequences derived from complementarity determining regions (CDRs) providing antigenic specificity. Non-human immunoglobulin sequences can be obtained from various non-human organisms suitable for antibody production, including but not limited to rat, mouse, rabbit goat, and the like. Humanized antibodies are also meant to include fully human antibodies. Methods for obtaining fully human antibodies, such as using for example phage display library systems or human MHC locus transgenic mice, are well known in the art (see, for example, U.S. Pat. Nos. 5,585,089; 5,530,101; 5,693,762; 6,180,370; 6,300,064; 6,696,248; 6,706,484; 6,828,422; 5,565,332; 5,837,243; 6,500,931; 6,075,181; 6,150,584; 6,657,103; 6,162,963). Such non-naturally occurring antibodies can be constructed using solid phase peptide synthesis, can be produced recombinantly or can be obtained, for example, by screening combinatorial libraries consisting of variable heavy chains and variable light chains as described by Huse et al. (Science 246:1275-1281 (1989)). These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); Borrabeck, Antibody Engineering, 2d ed. (Oxford University Press 1995)).

Antibodies specific for an antigen can be raised using an immunogen such as an isolated TIM polypeptide, or a fragment thereof, which can be prepared from natural sources or produced recombinantly, or an antigenic portion of the antigen that can function as an epitope. Such epitopes are functional antigenic fragments if the epitopes can be used to generate an antibody specific for the antigen. A non-immunogenic or weakly immunogenic antigen or portion thereof can be made immunogenic by coupling the hapten to a carrier molecule such as bovine serum albumin (BSA) or keyhole limpet hemocyanin (KLH). Various other carrier molecules and methods for coupling a hapten to a carrier molecule are well known in the art (see, for example, Harlow and Lane, supra, 1988). An immunogenic peptide fragment of an antigen can also be generated by expressing the peptide portion as a fusion protein, for example, to glutathione S transferase (GST), polyHis, or the like. Methods for expressing peptide fusions are well known to those skilled in the art (Ausubel et al., Current Protocols in Molecular Biology (Supplement 47), John Wiley & Sons, New York (1999)).

A TIM targeting molecule can be expressed recombinantly, as disclosed herein, as a polypeptide, a functional fragment of a polypeptide having a desired activity, or as a fusion polypeptide. Methods of making and expressing recombinant forrns of a TIM targeting molecule are well known to those skilled in the art, as taught, for example, in Sambrook et al., Molecular Cloning: A Laboratorv Manual, 2nd ed., Cold Spring Harbor Press, Plainview, N.Y. (1989); Ausubel et al., Current Protocols in Molecular Biology (Supplement 56), John Wiley & Sons, New York (2001); and Sambrook and Russel, Molecular Cloning: A Laboratorv Manual, 3rd ed., Cold Spring Harbor Press, Cold Spring Harbor (2001). Such methods are exemplified in the Examples, and FIG. 18 shows an exemplary expression vector for a TIM targeting molecule construct. One skilled in the art can readily determine a desired fragment, for example, a functional fragment of a TIM having a desired function, for example, the extracellular domain or a fragment thereof such as the Ig domain and/or mucin domain, for use as a TIM targeting molecule.

As discussed above, a TIM targeting molecule or agent can be a small molecule, a peptide, a polypeptide, a polynucleotide, including antisense and siRNAs, a carbohydrate including a polysaccharide, a lipid, a drug, as well as mimetics, and the like. Methods for generating such molecules are well known to those skilled in the art (Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); Gordon et al., J. Med. Chem. 37: 1233-1251 (1994); Gordon et al., J. Med. Chem. 37: 1385-1401 (1994); Gordon et al., Ace. Chem. Res. 29:144-154 (1996); Wilson and Czamik, eds., Combinatorial Chemistry: Synthesis and Application, John Wiley & Sons, New York (1997)). Methods for selecting and preparing antisense nucleic acid molecules are well known in the art and include in silico approaches (Patzel et al., Nucl. Acids Res. 27:4328-4334 (1999); Cheng et al., Proc. Natl. Acad. Sci. USA 93:8502-8507 (1996); Lebedeva and Stein, Ann. Rev. Pharmacol. Toxicol. 41:403-419 (2001); Juliano and Yoo, Curr. Opin. Mol. Ther. 2:297-303 (2000); and Cho-Chung, Pharmacol. Ther. 82:437-449 (1999)). Methods for producing si RNAs and using RNA interference have been described previously (Fire et al., Nature 391:806-811 (1998); Hammond et al. Nature Rev. Gen. 2: 110-119 (2001); Sharp, Genes Dev. 15: 485-490 (2001); and Hutvagner and Zamore, Curr. Opin. Genetics & Development 12:225-232( 2002); Hutvagner and Zamore, Curr. Opin. Genetics & Development 12:225-232 (2002); Bernstein et al., Nature 409:363-366 (2001); (Nykanen et al., Cell 107:309-321 (2001)).

The invention also provides a method of prophylactic treatment of a disease by administering to an individual a composition comprising an antigen and a TIM targeting molecule or agent in a pharmaceutically acceptable carrier. Thus, a composition of the invention can be used as a vaccine to prevent the onset of a disease or to decrease the severity of a disease. The method can be used for a variety of diseases, including but not limited to an infectious disease or cancer.

The invention additionally provides a method of ameliorating a sign or symptom associated with a disease by administering to an individual a composition comprising an antigen and a TIM targeting molecule or agent in a pharmaceutically acceptable carrier. The method can be used to decrease the severity of a disease. Thus, the compositions of the invention can be used therapeutically to treat a disease. One skilled in the art can readily determine a sign or symptom associated with a particular disease and the amelioration of an associated sign or symptom. The method can be used for a variety of diseases, including but not limited to an infectious disease or cancer. In the case of an infectious disease, the method can be used to decrease the amount of infectious agent in an individual having an infection.

The invention additionally provides a method of targeting a tumor. The method can include the steps of administering a TIM targeting molecule to a subject, wherein the tumor expresses a TIM or TIM ligand. The tumor can be, for example, a carcinoma, sarcoma and lymphoma. In another embodiment, the invention provides a method of inhibiting tumor growth by administering a TIM targeting molecule to a subject, wherein the tumor expresses a TIM or TIM ligand. In yet another embodiment, the invention provides a method of detecting a tumor by administering a TIM targeting molecule conjugated to a diagnostic moiety to a subject, wherein the tumor expresses a TIM or TIM ligand.

In still another embodiment, the invention provides a method of ameliorating a sign or symptom associated with an autoimmune disease by administering a TIM targeting molecule to a subject, as disclosed herein. The autoimmune disease can be, for example, rheumatoid arthritis, multiple sclerosis, autoimmune diabetes mellitus, systemic lupus erythematosus, psoriasis, psoriatic arthritis, an inflammatory bowel disease, such as Crohn's disease or ulcerative colitis, myasthenia gravis and autoimmune lymphoproliferative syndrome (ALPS), as well as atherosclerosis and Alzheimer's disease, or other autoimmune diseases, as disclosed herein. Autoimmune disorders are mediated by cellular effectors, for example, T cells, macrophages, B cells and the antibodies they produce, and others cells. These cells express one or more TIM or TIM ligands, as disclosed herein. By seliminating the cells involved in an autoimmune response, for example, using a lytic Fc in an antibody or fusion protein, or by using a toxic conjugate, a therapeutic benefit is achieved in such an autoimmune disorder.

In methods of the invention, a TIM targeting molecule can be administered alone or optionally administered with an antigen. In a method of the invention in which an immune response is stimulated, the TIM targeting molecule can enhance an immune response against an endogenous antigen or antigens or against an exogenous antigen or antigens administered with the TIM targeting molecule, as disclosed herein. For example, the antigen can be a tumor antigen in a method targeting a tumor. Similarly, an antigen associated with a cell mediating an autoimmune disease can be administered with a TIM targeting molecule or conjugate thereof, if desired. The TIM targeting molecule can also be conjugated with a therapeutic moiety. In addition, the TIM targeting molecule or TIM targeting molecule conjugate can be a TIM-Fc fusion polypeptide. Such a TIM-Fc fusion polypeptide can be target-cell depleting (lytic) or non target-cell depleting (non-lytic).

It is understood that modifications which do not substantially affect the activity of the various embodiments of this invention are also provided within the definition of the invention provided herein. Accordingly, the following examples are intended to illustrate but not limit the present invention.

### EXAMPLE I

### Purification of Anti-TIM-1 Antibodies

Hybridomas secreting mouse anti-human TIM-1 antibodies or rat anti-mouse TIM-1 antibodies were initially cultured in cell culture flasks and subsequently transferred to Bioperm cell culture reactors. Culture supernatants containing secreted antibodies were harvested every 48 hours, clarified, and stored at 4° C. The collected supernatants were pooled, and anti-TIM-1 antibodies were purified from the supernatants by Protein G Sepharose affinity chromatography and eluted from the column using glycine, pH 2.5-3.5. The eluates were pH neutralized and dialyzed against phosphate buffered saline (PBS). Purified antibodies were stored at −80° C. until further use.

### EXAMPLE II

### Construction of DNA Vectors for Murine and Human TIM-1/Fc Fusion Protein Expression

A shuttle plasmid vector (pTPL-1) for the cloning of the TIM-1/Fc fusion protein gene segments was designed and constructed. The basic vector, pTPL-1, carries bacterial and eukaryotic resistance genes as well as a multiple cloning site flanked by a CMV enhancer and a β-globin poly A site (see also FIG. 18 with TIM-3 fusion). The mouse non-lytic IgG2a/Fc fragment (hinge, CH2 and CH3 domains) was generated by oligonucleotide site-directed mutagenesis to replace the C1q binding motif and inactivate the FcγR1 binding sites (Zheng et al., J. Immunol. 154:5590-5600 (1995)).

The Fc region that can be part of the agents of the invention can be “lytic” or “non-lytic.” A non-lytic Fc region typically lacks a high affinity Fc receptor binding site and a C′1q binding site. The high affinity Fc receptor binding site of murine IgG Fc includes the Leu residue at position 235 of the IgG Fe. Thus, the murine Fc receptor binding site can be destroyed by mutating or deleting Leu 235. For example, substitution of Glu for Leu 235 inhibits the ability of the Fc region to bind the high affinity Fc receptor. The murine C′1q binding site can be functionally destroyed by mutating or deleting the Glu 318, Lys 320, and Lys 322 residues of the IgG. For example, substitution of Ala residues for Glu 318, Lys 320, and Lys 322 renders IgG Fc unable to direct antibody-dependent complement lysis. In contrast, a lytic IgG Fc region has a high affinity Fc receptor binding site and a C′1q binding site. The high affinity Fc receptor binding site includes the Leu residue at position 235 of IgG Fc, and the C′1q binding site includes the Glu 318, Lys 320, and Lys 322 residues of the IgG. Lytic IgG Fc has wild-type residues or conservative amino acid substitutions at these sites. Lytic IgG Fc can target cells for antibody dependent cellular cytotoxicity or complement directed cytolysis (CDC). Appropriate mutations for human IgG are also known (see, for example, Morrison et al., The Immunologist 2:119-124 (1994); and Brekke et al., The Immunologist 2:125 (1994)).

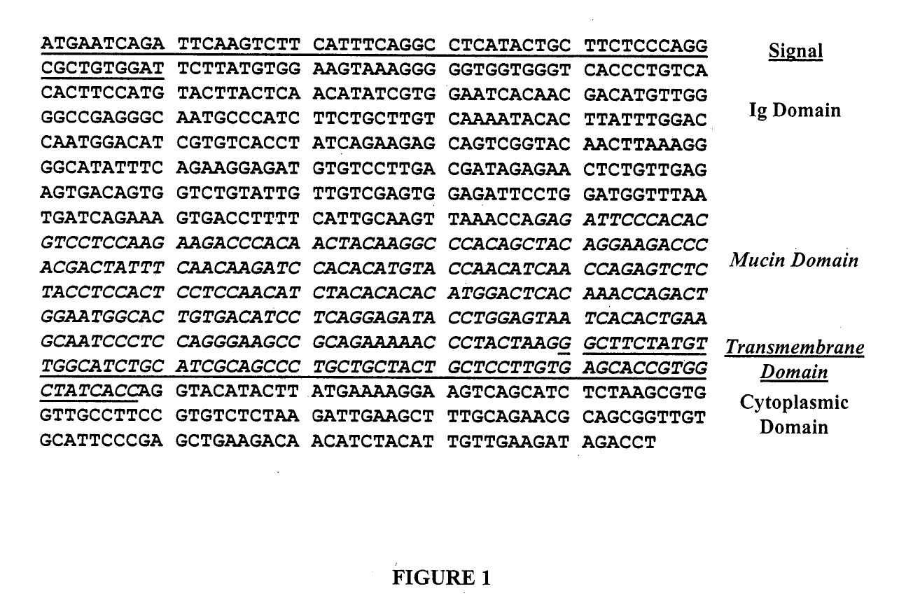

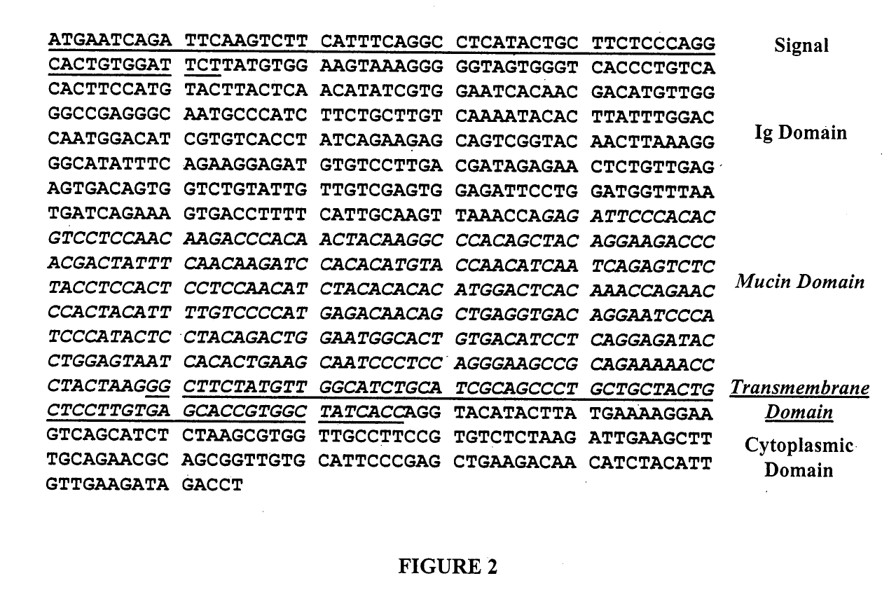

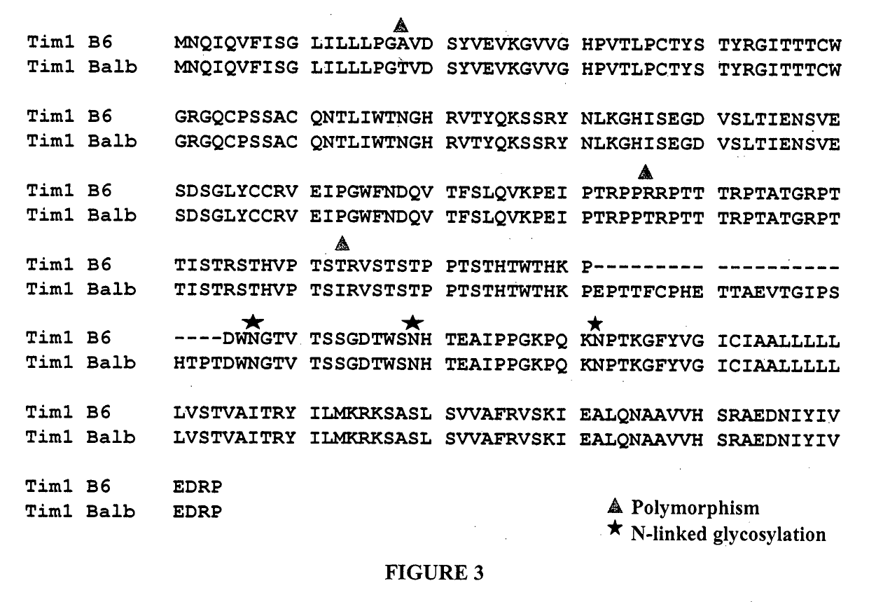

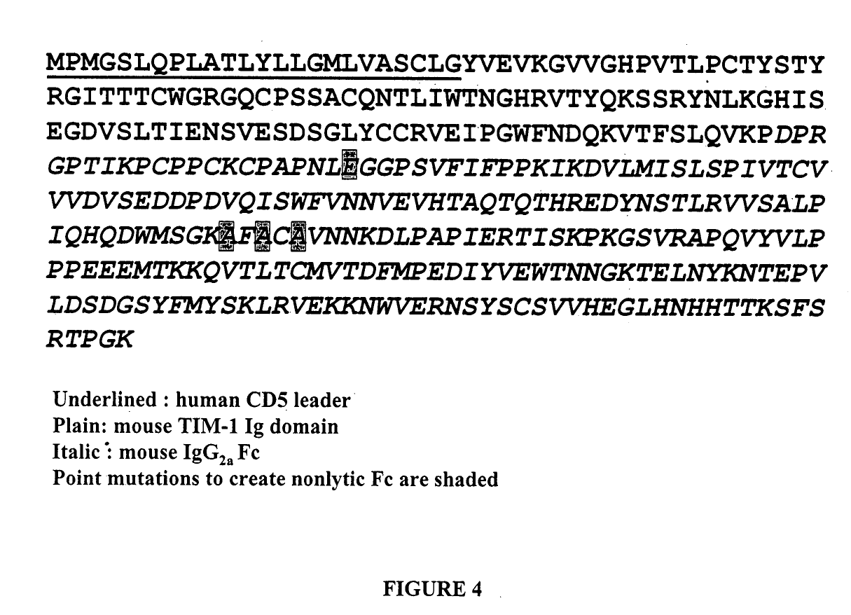

Both the wild-type and point-mutated IgG2a Fc fragments were amplified by PCR, respectively, and cloned into pTPL-1 to create pTPL-1/mFc2a and pTPL-1/mFc2a/n1 (n1, nonlytic). Subsequently, the human CD5 signal sequence gene segment was synthesized by annealing and fill-in reactions using the two following oligonucleotides (Locus: NM_{—}014207, forward oligonucleotide: 5′-TGGCACCGGTGCCACCATGCCCATGGGGTCTCTGCAACCGCTGGCCACCT T GTACCTGCTGGGG-3′, SEQ ID NO:43; and reverse oligonucleotide: 5′-TAGGAGATCTCCTAGGCAGGAAGCGACCAGCATCCCCAGCAGGTACAAG GTGGCCAGCGG-3′, SEQ ID NO:44). The forward oligonucleotide contains a suitable restriction site and a Kozac consensus sequence prior to the initiating ATG (underlined) of the CD5 signal sequence and the 5′ end of this sequence. The reverse oligonucleotide is composed of sequences derived form the 3′ end of the CD5 signal sequence and suitable restriction sites. The synthesized gene fragment was digested and cloned into the pTPL-1/Fc vectors. This created the plasmids pTPL-1/CD5/mFc2a and pTPL-1/CD5/mFc2a/n1. Finally, the respective extracellular domains of mouse TIM-1 were PCR-amplified and cloned into pTPL-1/CD5/mFc2a and pTPL-1/CD5/mFc2a/n1 vectors, between the human CD5 signal sequence and the Ig Fc regions. This cloning step yielded the final expression plasmids pTPL-1/TIM-1 Fc and pTPL-1/TIM-1 Fc/n1. The accuracy of the plasmid constructs was confirmed by DNA sequencing. The following mouse TIM-1/Fc expression vectors were constructed: (1) Immunoglobulin (Ig) domain of TIM-1 alone fused to non-lytic and lytic mouse IgG2a Fc. The respective nucleotide sequence of the Ig domain is given in FIGS. 1 and 2. (2) Full length extracellular domain of mouse TIM-1 (either BALB/c or C57B1/6 allele) fused to non-lytic and lytic mouse IgG2a Fc. The sequences of the extracellular domains (Ig domain+mucin domain) are given in FIGS. 1 and 2. The protein sequence is given in FIG. 2. The protein sequence of an exemplary TIM-1/Fc fusion protein is given in FIG. 4.

In a fashion analogous to the above described mouse TIM-1/Fc expression vectors, vectors expressing human TIM-1/Fc were also generated. To do so, either human IgG1 Fc or human IgG4 Fc (hinge, CH2 and CH3 domains of the respective immunoglobulin) were amplifed by PCR and cloned into pTPL-1. A CD5 leader sequence was then inserted as described above, and finally different TIM-1 alleles as described in US patent application 20030124114 were cloned into the expression vector. Again, vectors containing either the Ig domain of TIM-1 alone or the Ig and mucin domains of TIM-1 were used to generate the TIM-1/Fc expression vectors. Similar constructs were made for TIM-3 and TIM-4 as well as mouse TIM-2.

### EXAMPLE III

### Transient Expression of TIM/Fc Fusion Proteins in 293 Cells

To test the functionality of the expression vectors generated, transient transfections in 293 cells were performed. Briefly, 80-90% confluent 293 cells in serum-free growth medium (293-SFM II; InvitroGen, Carlsbad, Calif.) were transfected using the Lipofectamine 2000 system according to the manufacturer's instructions (InvitroGen, Carlsbad, Calif.). Routinely, 1 μg of plasmid DNA per 10⁵ cells was used. One day after transfection, the growth medium was replaced with fresh medium and the cells cultured for up to 7 days. Cell culture supernatants were clarified by centrifugation and TIM-1/Fc or TIM-3/Fc fusion protein purified by Protein G Sepharose affinity chromatography. After low pH elution from the Protein G beads, the purified protein were dialyzed against PBS and stored at −80° C. The identity, purity and integrity of the proteins produced were analyzed by sodium dodecyl sulfate polyacrylamide gel electrophoresis (SDS PAGE) and silver or Coomassie staining, Western blotting and ELISA.

### EXAMPLE IV

### Generation of CHO Cell Lines Stably Expressing TIM-1/Fc, TIM-3/Fc and TIM-4/Fc

CHO cell lines stably expressing the various TIM-1/Fc fusion proteins were generated as follows: Adherent (CHO-K1) or suspension-growth CHO-S cells (InvitroGen, Carlsbad, Calif.) were transfected with the appropriate expression plasmid (pTPL-1; TIM-1/Fc series) using either a commercially available kit (Lipofectamine 2000, InvitroGen, Carlsbad, Calif.) and according to the manufacturer's instructions or by electroporation. The transfected cells were allowed to recover for one day in growth medium (CHO-SFM II; InvitroGen, Carlsbad, Calif.; or DMEM, 10% fetal calf serum) and were then transferred into selection medium containing the antibiotic G418 (0.5 mg/ml to 1 mg/ml). Individual clones were generated by single-cell limiting dilution cloning (suspension lines) or by “clone picking” (adhering cell lines) and further propagated. ELISAs were used to assay culture media supernatants for the presence of secreted TIM-1/Fc proteins. High producing clones were further sub-cloned and expanded for protein production. Essentially identical protocols were used to generate CHO cell lines stably expressing TIM-3/Fc and TIM-4/Fc fusion proteins.

### EXAMPLE V

### Production and Purification of Mouse TIM/Fc Fusion Protein

Stable CHO cell lines expressing TIM-1/Fc fusion protein were expanded in serum free growth medium (CHO-SFM II; InvitroGen, Carlsbad, Calif.) or DMEM, 5% fetal calf serum. Culture media were collected, clarified by centrifugation and/or filtration, concentrated by ultra filtration (Pall Ultrasette™, Ann Arbor, Mich.) and immobilized via Protein A or G. The protein-bound resin was washed and TIM-1/Fc fusion protein eluted by low pH. Fractions were collected and adjusted to neutral pH. As necessary, the eluted TIM-1/Fc proteins were further purified by ion exchange chromatography and size exclusion chromatography. Purified protein was dialyzed against a suitable physiological buffer, for example, PBS, and stored in aliquots at −80° C. Essentially identical protocols were used to produce and purify TIM-3/Fc and TIM-4/Fc fusion proteins.

### EXAMPLE VI

### Anti-TIM-1 as an Adjuvant for Hepatitis B Vaccination

BALB/c mice were vaccinated with a single dose (10 micrograms, “mcg”) of Engerix-B™ vaccine (Glaxo Smith Kline) with or without 50 mcg/ml anti-TIM-1 antibody. Antibodies were admixed with the vaccine (vaccine contains 0.5 mg/ml aluminum hydroxide as an adjuvant) prior to injection. Control mice were treated with aluminum hydroxide in PBS, PBS alone, or vehicle containing isotype matched antibody controls. On days 7, 14, and 21 after immunization, mice from each group were taken for analysis. Briefly, spleens and serum were harvested, processed into a single cell suspension in RPMI media supplemented with β-mercaptoethanol, 10% fetal bovine serum (FBS) and antibiotics (penicillin, streptomycin, fungizone). Processed spleen cells (3×10⁵ cells) were incubated in the presence of purified hepatitis B surface antigen (5 mcg/ml, Research Diagnostics, Inc., Flanders, N.J.). After incubation for 96 hours at 37° C., 5% CO₂, total viable cells were analyzed by the WST-cell proliferation kit (Roche Diagnostics, Indianapolis, Ind.). In addition, supernatants from these experimental wells were harvested after 96 hours and analyzed for the presence of IFN-γ and IL-4 using a commercial cytokine ELISA kit according to the manufacturer's instructions (R&D Systems; Minneapolis Minn.). Serum samples were diluted to 1:200 and analyzed in an ELISA that detects antibodies specific for hepatitis B surface antigen.

In other experiments, spleen cells isolated from vaccinated animals were incubated with 0.3, 1.0, or 3.0 mcg/ml of hepatitis B surface antigen in the manner described above. Proliferation of cells in response to antigen was measured using a Delfia Proliferation Assay kit (Perkin Elmer, Boston, Mass.). Briefly, BALB/c mice (6 mice per group) were vaccinated with Engerix B™ adjuvanted with alum and 100 mcg of TIM-1 antibody. Proliferation of hepatitis B surface antigen-specific spleen cells was measured by incubating lymphocyte preparations for 4 days in the presence or absence of antigen in a total volume of 0.2 ml complete media (RPMI 10% Fetal Bovine Serum, penicillin-streptomycin, β-mercaptoethanol). Twenty-four hours prior to the end of each proliferation time point, cells in 96-well flat bottom tissue culture plates were labeled with 0.02 ml of 5-bromo-2′-deoxyuridine (BrdU) Labeling Solution. After 24 hours, the plates were centrifuged and media removed. Nucleic acid contents of the wells were fixed to the plastic and anti-BrdU antibodies, labeled with europium, were added to bind the incorporated BrdU. After washing the wells and addition of a fluorescence inducer, europium fluorescence was analyzed using a Wallac Victor 2 multilable analyzer and expressed as relative fluorescence units (RFU). Assay controls included wells without cells, cells without BrdU, and cells without antigenic stimulation.

Experimental results show that administration of a commercial Hepatitis B vaccine (Engerix-B™, GlaxoSmithKline) is only poorly immunogenic in mice. This vaccine does not elicit a cell-mediated immune response in mice, and antibodies against Hepatitis B antigen are only detected three weeks after immunization. Administration of anti-TIM-1 antibody as an adjuvant at the time of vaccination with the Hepatitis B vaccine led to the generation of an antigen-specific cell mediated immune response against Hepatitis B antigen within seven days after vaccination. Cell mediated immunity has been assayed by monitoring immune cell proliferation after re-exposure to antigen and by measuring the production of T helper cytokines. Administration of anti-TIM-1 antibody as an adjuvant at the time of vaccination also led to the generation of antibodies against Hepatitis B antigen within seven days after vaccination.

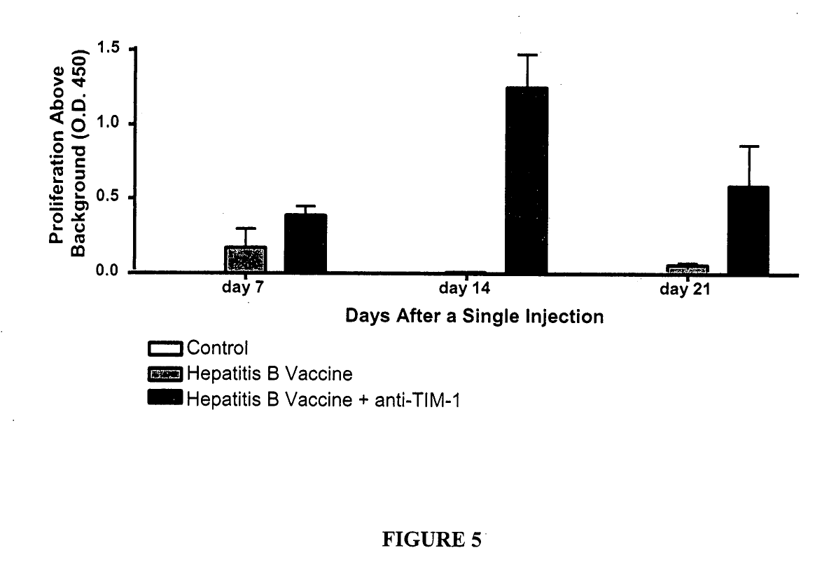

FIG. 5 shows proliferation to antigen upon re-stimulation. BALB/c mice were vaccinated with Engerix-B™ (10 mcg) alone or with a single dose of anti-TIM antibody (50 mcg). At the indicated times, the spleens were analyzed for proliferation to Hepatitis B surface antigen (96 h assay). Whereas vaccine alone stimulated little splenocyte and T cell proliferation in response to antigen, anti-TIM-1 antibody greatly enhanced the cellular proliferative response to antigen, indicating increased cellular immunity. These results show that anti-TIM-1 antibodies improved the response to hepatitis B vaccine.

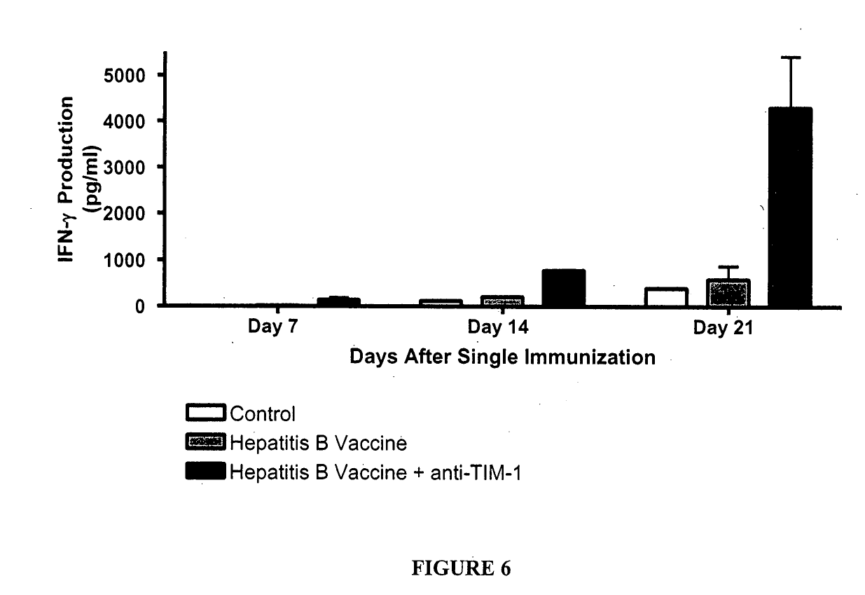

FIG. 6 shows the production of cytokines after re-stimulation with antigen. BALB/c mice were immunized with 10 mcg of Hepatitis B vaccine, or with 10 mcg vaccine with anti-TIM antibodies. At days 7, 14, and 21, spleen cells were stimulated in vitro with Hepatitis B antigen. After 96 hours, the supernatants were analyzed for IFN-γ and IL-4, respectively. Whereas vaccine alone stimulated little IFN-γ production (a Th1 cytokine) in response to antigen, anti-TIM-1 antibody greatly enhanced the production of this cytokine, indicating an increased Th1 response. In contrast, expression of IL-4, a Th2 cytokine, was at background levels for all time points. These results show anti-TIM-1 antibody adjuvant effects on Interferon-γ production.

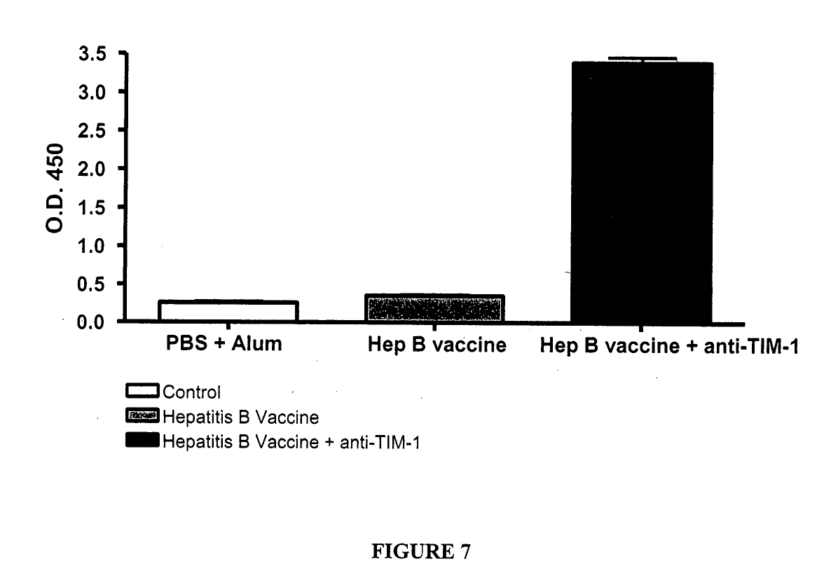

FIG. 7 shows the production of hepatitis B specific antibodies. Serum samples from mice vaccinated with Hepatitis B vaccine with or without anti-TIM antibodies (single dose; 50 mcg) were tested for the presence of antibodies specific for Hepatitis B surface antigen on day 7 after immunization. Whereas vaccine alone stimulated little antibody response against Hepatitis B antigen early after immunization, anti-TIM-1 antibody stimulated a strong antibody response. These results show that treatment with anti-TIM-1 antibody in combination with hepatitis B vaccine induces antibodies to hepatitis B antigen.

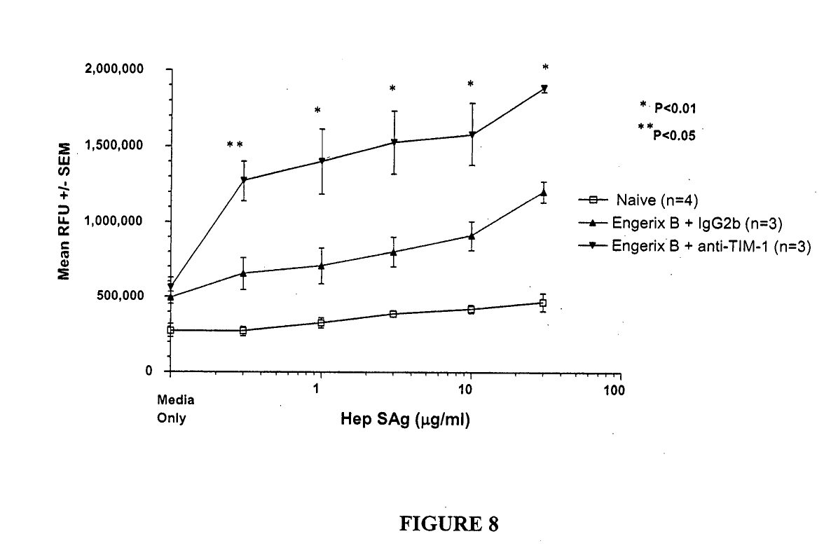

FIG. 8 shows the proliferation of hepatitis B surface antigen-specific splenocytes in a dose dependent relationship with antigen stimulation. Splenocytes from mice vaccinated once with 10 mcg of Engerix B™, with or without 100 mcg TIM-1 mAbs, were isolated and cultured in the presence or absence of increasing hepatitis B surface antigen concentrations. After 4 days of incubation, the wells were analyzed for proliferation using the Delfia Cell Proliferation Assay. Mice that received vaccine with TIM-1 mAbs produced a statistically significant response (p<0.05) against specific antigen versus vaccination with the Engerix B™ vaccine alone or with the isotype control antibody. These results show that anti-TIM-1 enhances proliferation of splenocytes against hepatitis B surface antigen.

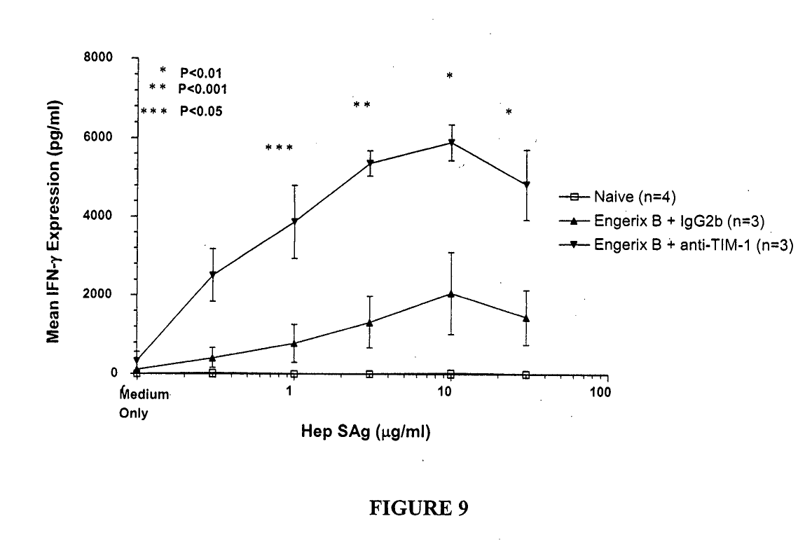

FIG. 9 show the production of IFN-γupon stimulation with specific antigen. Interferon-γexpression was measured in whole splenocytes against hepatitis B surface antigen (HepBsAg). Supernatants from the proliferation assay wells described above were removed for cytokine analysis by ELISA. Mice that received vaccine with TIM-1 mAbs produced a significantly higher amount of IFN-γ (p<0.05) in response to antigen stimulation than did the mice that received vaccine alone or vaccine with the isotype control antibody. No IL-4 was detectable. These results show that anti-TIM-1 enhances IFN-γ expression in response to hepatitis B surface antigen.

### EXAMPLE VII

### Anti-TIM-1 as an Adjuvant for HIV Antigens

Six to eight week old C57BL/6 mice (4 per group) were vaccinated subcutaneously with a single dose of HIV p24 antigen (25 or 50 mcg) in PBS and intraperitoneally with either 50 or 100 mcg TIM-1 mAb, isotype control antibody, or 50 or 100 mcg CpG 1826 (synthesized by Invitrogen Corporation; Carlsbad Calif.) oligodeoxy-nucleotides on days 1 and 15. The CpG 1826 oligo is

| | |
|---|---|
| TCCATGACGTTCCTGACGTT | (SEQ ID NO:45) |
| ZOOFZEFOEZZOOZEFOEZT | |

The top line is the sequence of the nucleotides in the standard 1-letter abbreviation nomenclature. All of the bases, except for the final T, are modified by phosphorothioation. The second line is the sequence using 1-letter abbreviations for phosphorothioated bases. The code is F=A-phosphorothioate, O=c-phosphorothioate, E=g-phosphorothioate, Z=T-phosphorothioate. Mice were then sacrificed on day 21 and the spleen cells were harvested for measuring proliferation to antigen. Briefly, spleen cells were measured by incubating lymphocyte preparations for 4 days in the presence or absence of HIV p24 antigen in a total volume of 0.2 ml complete media (RPMI 10% Fetal Bovine Serum, penicillin-streptomycin, β-mercaptoethanol). Cell proliferation was determined using the Delfia Cell Proliferation Assay (PerkinElmer,). Twenty-four hours prior to the end of the incubation period, cells in 96-well round bottom tissue culture plates were labeled with 0.02 ml of BrdU Labeling Solution. After 24 hours, the plates were centrifuged and media removed. Nucleic acid contents of the wells were fixed to the plastic and anti-BrdU antibodies, labeled with europium, were added to bind the incorporated BrdU. Incorporation of BrdU was expressed as relative fluorescence units (RFU) of europium using a fluorimetric analyzer. Assay controls included wells without cells, cells without BrdU, and vehicle alone (phosphate buffered saline, PBS).

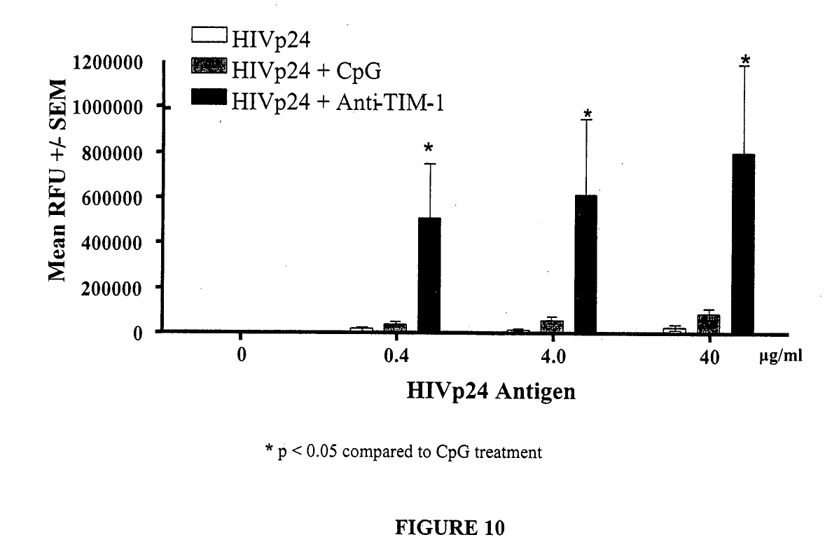

FIG. 10 shows that mice immunized with HIV p24 antigen plus TIM-1 mAb yielded a significantly higher proliferative response (p<0.05 compared to CpG) to antigen compared to either the isotype control antibody or the CpG oligonucleotides. Mice were vaccinated subcutaneously with a single dose of HIV p24 antigen (50 mcg) in PBS and intraperitoneally with either 100 mcg TIM-1 mAb, isotype control antibody, or 100 mcg CpG (1826) oligodeoxy-nucleotides on days 1 and 15. Mice were then sacrificed on day 24 and the spleen cells were harvested for proliferation to antigen. These results show that anti-TIM-1 enhances proliferative response to HIV p24 antigen.

### EXAMPLE VII

### Anti-TIM-1 as an Adjuvant for Influenza Vaccination

BALB/c mice were vaccinated with a single dose (30 mcg) of Fluvirin™ vaccine (Evans Vaccines, Ltd) with or without 50 mcg/ml anti-TIM-1 antibody. Antibodies were admixed with the vaccine just prior to injection. Control mice were treated with PBS alone, or PBS containing isotype matched antibody controls. On day 10 after immunization, mice from each group were taken for analysis. Briefly, spleens and serum were harvested, processed into a single cell suspension in RPMI media supplemented with β-mercaptoethanol, 10% FBS and antibiotics (penicillin, streptomycin, fungizone). Processed spleen cells (3×10⁵ cells) were incubated in the presence of inactivated whole influenza (1 mcg/ml, Beijing strain, H1N1; Research Diagnostics, Inc., Flanders, N.J.). After incubation for 96 hours at 37° C., 5% CO₂, viable cells were analyzed by the WST-cell proliferation kit (Roche Diagnostics, Indianapolis, Ind.). Supernatants from these experimental wells were harvested after 96 hours and analyzed for the presence of IFN-γ and IL-4 using a commercial cytokine ELISA kit according to the manufacturer's instructions (R&D Systems). Serum samples were diluted to 1:200 and analyzed in an ELISA that detects antibodies specific for influenza virus.

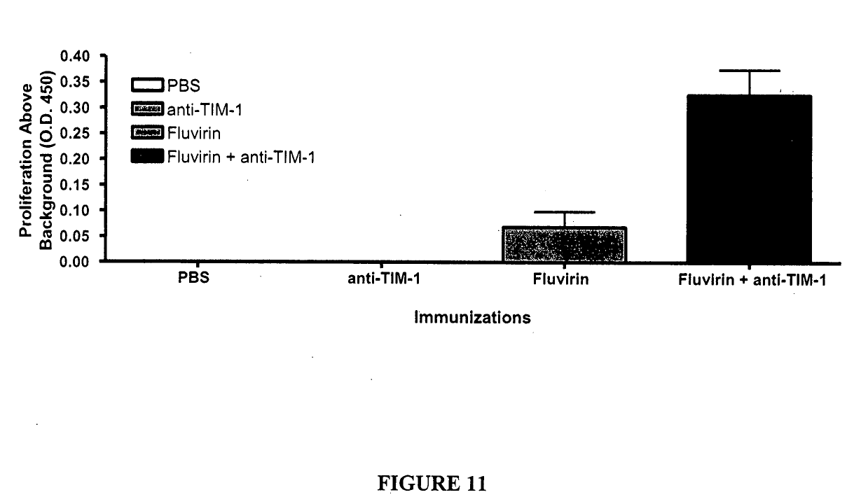

FIG. 11 shows the proliferative response of splenocytes to influenza antigen. BALB/c mice were immunized with the influenza vaccine Fluvirin™ or Fluvirin™+anti-TIM-1 antibodies (single dose; 50 mcg). Ten days later, the response to stimulation by virus (H IN I) was measured in a 96 h proliferation assay. PBS, and the anti-TIM-1 antibody alone were treatment controls. Whereas vaccine alone stimulated little splenocyte and T cell proliferation in response to antigen, anti-TIM-1 antibody greatly enhanced the cellular proliferative response to antigen, indicating increased cellular immunity. These results show anti-TIM-1 antibody adjuvant effects for influenza vaccination.

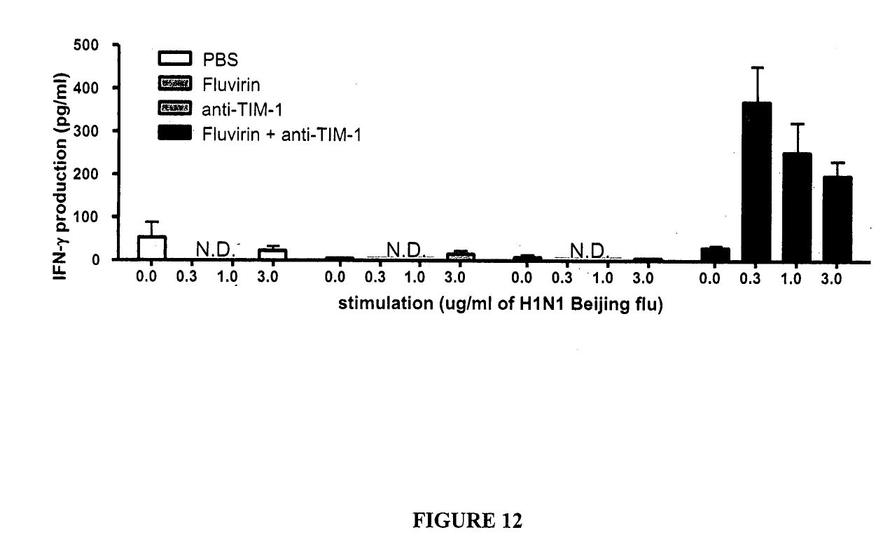

FIG. 12 shows the cytokine production from influenza-immunized mice. BALB/c mice were immunized with 30 mcg of the influenza vaccine Fluvirin™ or Fluvirin™+anti-TIM antibodies (single dose; 50 mcg). After 10 days, splenocytes were prepared and the production of Th1 (IFN-γ) and Th2 (IL-4) cytokines upon re-stimulation with virus (H1N1) was determined after 96 h in culture (PBS, Fluvirin™, anti-TIM-1, and Fluvirin™+anti-TIM-1 shown left to right in FIG. 12). Whereas vaccine alone stimulated little IFN-γ production (a Th1 cytokine) in response to antigen, anti-TIM-1 antibody greatly enhanced the production of this cytokine, indicating an increased Th1 response. IL-4 production was at or below background. Thus, in contrast to IFN-γ, expression of IL-4, a Th2 cytokine, was at background levels. These results show that anti-TIM-1 adjuvant elicits influenza-specific Th1 cytokine responses.

### EXAMPLE IX

### Anti-TIM-1 as Adjuvants to Generate Heterosubtypic Immune Responses Against Different Influenza Strains

BALB/c mice (3 per group) were vaccinated with a single dose (10 mcg) of Beijing influenza virus (A/Beijing/262/95, H1N1) with or without 100 mcg/ml anti-TIM-1 antibody. Antibodies were admixed with the antigen just prior to injection. Control mice were treated with PBS alone, or antigen containing isotype matched (rat IgG2b) antibody controls. On day 21 after immunization, mice from each group were taken for analysis. Briefly, spleens and serum were harvested and processed into a single cell suspension in RPMI media supplemented with β-mercaptoethanol, 10% FBS and antibiotics (penicillin, streptomycin, fungizone). Processed spleen cells (3×10⁵ cells) were incubated in the presence of inactivated whole influenza (1 mcg/ml, Beijing strain, H1N1 or A/Kiev-like 301/94-Johannesburg/33/94, H3N2; Research Diagnostics, Inc., Flanders, N.J.). After incubation for 96 hours at 37° C., 5% CO₂, viable cells were analyzed by the Delfia proliferation kit (PerkinElmer). Twenty-four hours prior to the end of the incubation period, cells in 96-well round bottom tissue culture plates were labeled with 20 μl of BrdU Labeling Solution. After 24 hours, the plates were centrifuged and media removed. Nucleic acid contents of the wells were fixed to the plastic and anti-BrdU antibodies, labeled with Europium, were added to bind the incorporated BrdU. Incorporation of BrdU was expressed as relative fluorescence units (RFU) of Europium using a fluorimetric analyzer. Assay controls included wells without cells, cells without BrdU, and cells without antigenic stimulation. Supernatants from these experimental wells were harvested after 96 hours and analyzed for the presence of IFN-γ and IL-4 using a commercial cytokine ELISA kit according to the manufacturer's instructions (R&D Systems).

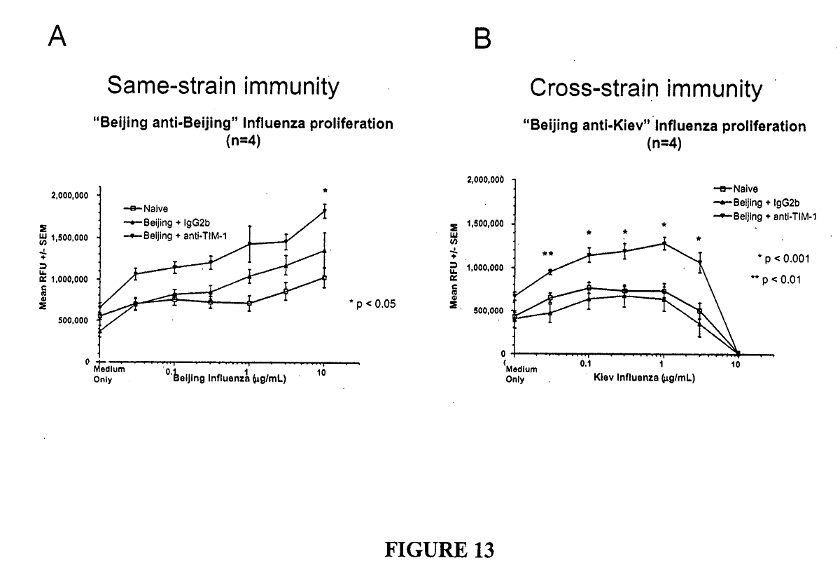

FIG. 13 shows the proliferative response of Beijing-immunized mice against stimulation by Beijing virus (A) or Kiev virus (B). BALB/c mice were immunized with 10 mcg inactivated Beijing influenza virus in the presence or absence of 100 mcg TIM-1 mAb or isotype control (rat IgG2b). After 21 days, the spleens were harvested for in vitro analyses. Proliferation is enhanced using TIM-1 mAbs and response to Kiev stimulation demonstrates cross-strain immunity (p<0.01). These results show that anti-TIM-1 enhances proliferation of splenocytes against influenza A and stimulates cross-strain immunity.

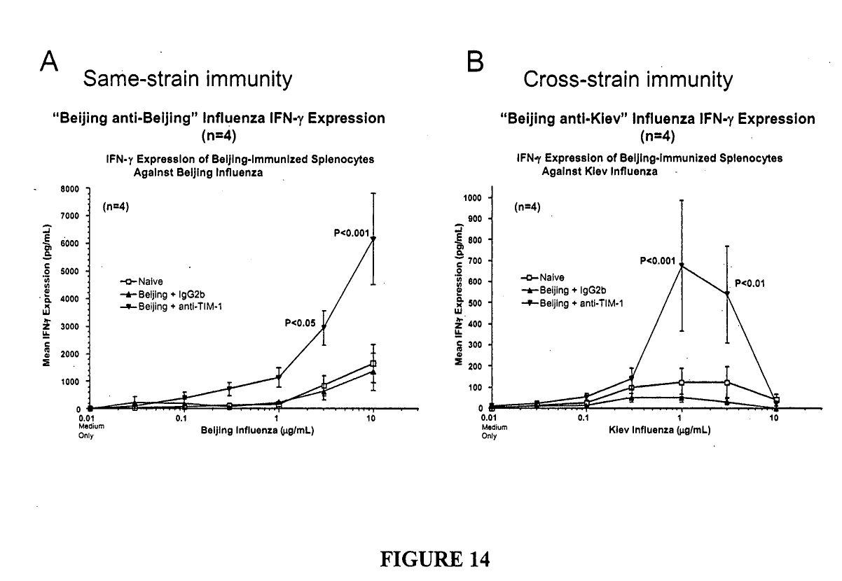

FIG. 14 shows the cytokine response of Beijing-immunized mice against stimulation by Beijing virus (A) or Kiev virus (B). BALB/c mice were immunized with 10 mcg inactivated Beijing influenza virus in the presence or absence of 100 mcg TIM-1 mAb or isotype control (rat IgG2b). After 21 days, the spleens were harvested for in vitro analyses. Supernatants from the proliferation assays were analyzed for the presence of IFN-γ. Panel A shows that addition of TIM-1 mAbs significantly (p<0.01) enhances the production of IFN-γ in response to Beijing virus (H1N1) stimulation. Panel B shows that the addition TIM-1 mAbs also significantly (p<0.01) enhances the production of IFN-γ in response to stimulation with the heterosubtypic Kiev strain (H3N2). These results show that anti-TIM-1 enhances cross-strain immunity.

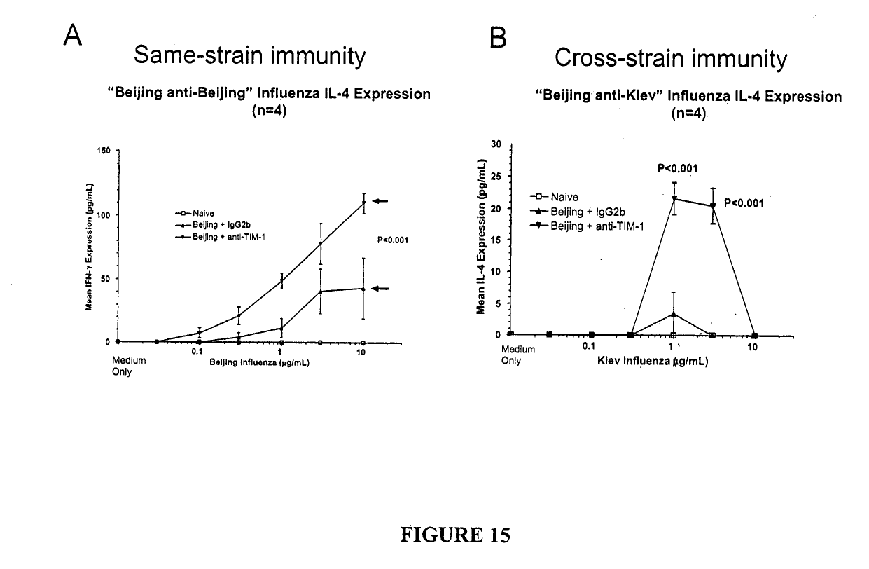

FIG. 15 shows the IL-4 cytokine production of Beijing-immunized mice against stimulation by Beijing virus (A) or Kiev virus (B). BALB/c mice were immunized with 10 mcg inactivated Beijing influenza virus in the presence or absence of 100 mcg TIM-1 mAb or isotype control (rat IgG2b). After 21 days, the spleens were harvested for in vitro analyses. Supernatants from the proliferation assays were analyzed for the presence of IL-4. Panel A shows that addition of TIM-1 mAbs significantly (p<0.01) enhances the production of IL-4 in response to Beijing virus (H1N1) stimulation. Panel B shows that the addition TIM-1 mAbs also significantly (p<0.01) enhances the production of IL-4 in response to stimulation with the heterosubtypic Kiev strain (H3N2). These results show that IL-4 expression was enhanced by anti-TIM-1 in splenocytes stimulated with influenza A.

### EXAMPLE X

### Anti-TIM-1 and Anti-TIM-3 as Adjuvants for Anthrax Vaccination

C57BL/6 mice were vaccinated with a single dose (40 mcg) of recombinant Protective Antigen (rPA, List Biological Laboratories; Campbell Calif.) with or without 50 mcg/ml anti-TIM-3 antibody. Antibodies were admixed with the antigen with 1.2 mg/ml aluminum hydroxide as an adjuvant just prior to injection. Control mice were treated with aluminum hydroxide in PBS or vehicle containing isotype matched antibody controls. On day 10 after immunization, mice from each group were taken for analysis. Briefly, spleens and serum were harvested, processed into a single cell suspension in RPMI media supplemented with P-mercaptoethanol, 10% FBS and antibiotics (penicillin, streptomycin, fungizone). Processed spleen cells (3×10⁵ cells) were incubated in the presence of rPA (1 mcg/ml, Research Diagnostics, Inc., Flanders, N.J.). After incubation for 96 hours at 37° C., 5% CO₂, viable cells were analyzed by the WST-cell proliferation kit (Roche Diagnostics, Indianapolis, Ind.). Additionally, supernatants from these experimental wells were harvested after 96 hours and analyzed for the presence of IFN-γ and IL-4 using a commercial cytokine ELISA kit according to the manufacturer's instructions (R&D Systems). Serum samples were diluted to 1:200 and analyzed in an ELISA that detects antibodies specific for rPA antigen.

Alternatively, C57BL/6 mice were vaccinated with a single dose (0.2 ml) of BioThrax™ (AVA; Bioport, Lansing, Mich.) with or without 50 mcg/ml anti-TIM-1 antibody. Antibodies were admixed with the antigen with 1.2 mg/ml aluminum hydroxide as an adjuvant just prior to injection. Control mice were treated with BioThrax™ vaccine alone or BioThrax™ vaccine containing isotype matched antibody controls. On day 7 after immunization, mice from each group were taken for analysis and blood serum samples collected. Serum samples were diluted to 1:200 and analyzed in an ELISA that detects antibodies specific for rPA antigen. In addition, spleens were harvested on day 15, processed into a single cell suspension in RPMI media supplemented with P-mercaptoethanol, 10% FBS and antibiotics (penicillin, streptomycin, fungizone). Processed spleen cells (3×10⁵ cells) were incubated in the presence of rPA (1 mcg/ml, Research Diagnostics, Inc., Flanders, N.J.). After incubation for 96 hours at 37° C., 5% CO₂, viable cells were analyzed by the WST-cell proliferation kit (Roche Diagnostics, Indianapolis, Ind.). Additionally, supernatants from these experimental wells were harvested after 96 hours and analyzed for the presence of IFN-γ and IL-4 using a commercial cytokine ELISA kit according to the manufacturer's instructions (R&D Systems).

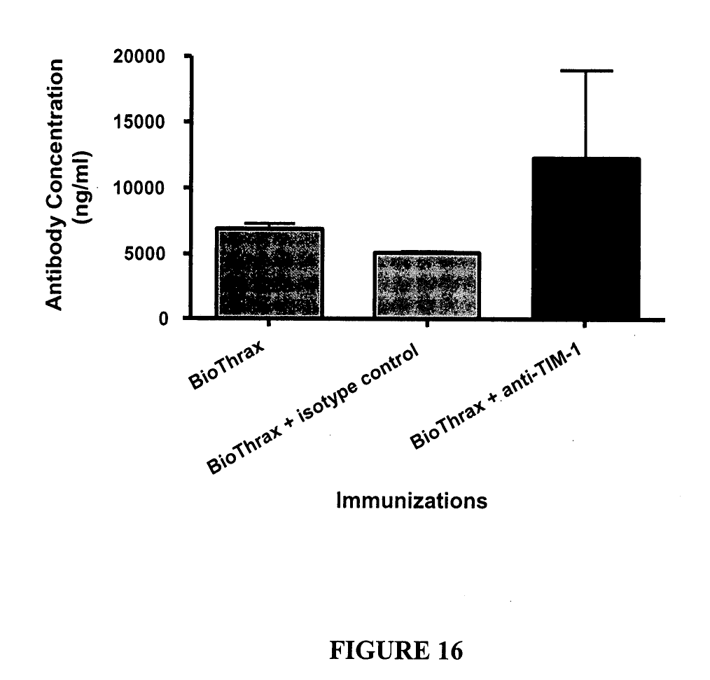

FIG. 16 shows the anti-rPA antibody response after vaccination. C57BL/6 mice were immunized with the 0.2 ml of AVA (Anthrax Vaccine Absorbed) BioThrax™ or BioThrax™+anti-TIM-1 antibodies. Seven days later, total serum antibodies specific for rPA were measured in an ELISA. BioThrax™ alone and BioThrax™+isotype matched antibody were treatment controls. Whereas vaccine alone stimulated little antibody response against anthrax antigen, anti-TIM-1 antibody stimulated a significantly elevated antibody response. These results show that BioThrax™+anti-TIM-1 increases antibody production.

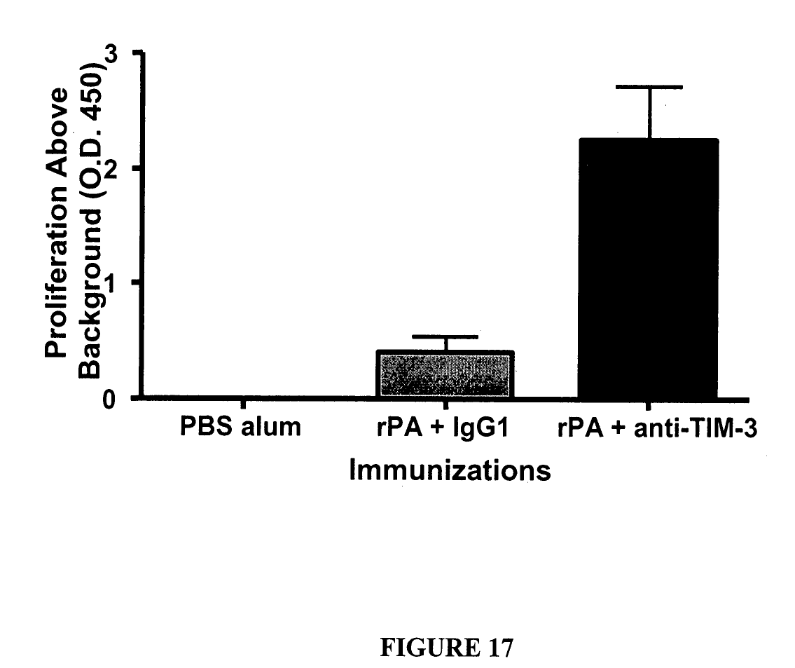

FIG. 17 shows anti-TIM adjuvant effects for anthrax vaccination. C57BL/6 mice were immunized with recombinant Protective Antigen (rPA; 40 mcg) or rPA+anti-TIM-3 antibodies (single dose; 50 mcg). Ten days later, the response of splenocytes to re-stimulation by rPA was measured in a 96 h proliferation assay. PBS and rPA+isotype matched control antibody were treatment controls. These results show anti-TIM-3 adjuvant effects for anthrax vaccination.

### EXAMPLE XI

### Anti-TIM-1 as an Adjuvant for Listeria Vaccination

C57BL/6 mice were vaccinated with a single dose of heat killed Listeria monocytogenes (HKLM) with or without 50 mcg/ml anti-TIM-1 antibody. Antibodies were admixed with the antigen and aluminum hydroxide (as adjuvant) prior to injection. Control mice were treated with aluminum hydroxide in PBS, PBS alone, or vehicle containing isotype matched antibody controls. On day 10 after immunization, mice from each group were taken for analysis. Briefly, spleens and serum were harvested, processed into a single cell suspension in RPMI media supplemented with β-mercaptoethanol, 10% FBS and antibiotics (penicillin, streptomycin, fungizone). Processed spleen cells (3×10⁵ cells) were incubated in the presence of 1 mcg/ml HKLM. After incubation for 96 hours at 37° C., 5% CO₂, viable cells were analyzed by the WST-cell proliferation kit (Roche Diagnostics, Indianapolis, Ind.). Supernatants from these experimental wells were harvested after 96 hours and analyzed for the presence of IFN-γ and IL-4 using a commercial cytokine ELISA kit according to the manufacturer's instructions (R&D Systems). Serum samples were diluted to 1:200 and analyzed in an ELISA that detects antibodies specific for HKLM.

### EXAMPLE XII

### TIM-1/Fc, TIM-4/Fc and Anti-TIM-1 as Adjuvants for Cancer Vaccines and as Therapeutic Agents for the Treatment of Tumors

C57BL/6 or BALB/c mice were subcutaneously injected with 10⁶ gamma-irradiated or mitomycin-treated B16.F10 (melanoma), EL4 (thymoma), or p815 (mastocytoma) cells. At the time of vaccination with inactivated tumor cells, the animals were also treated with 0.1 mg rat anti-mouse TIM-1 or TIM-1/Fc, either subcutaneously or intraperitoneally. Control mice were treated with an equal amount of rat or mouse IgG2a. This vaccination protocol was repeated after 14 days. On day 20, the mice were challenged with 10⁵ to 10⁶ live tumor cells (titrated for each tumor type to yield 100% tumor incidence without treatment: B16.F10: 5×10⁵ cells; P815 and EL4: 10⁶ cells) and tumor incidence and size monitored on a bi-daily basis.

The mice and cell lines employed in the experiments were C57BL/6, DBA/2 or BALB/c female mice, aged 8-10 weeks at the time of delivery. EL4 thymoma, B16F10 melanoma and P815 mastocytoma tumor cells were purchased from American Type Culture Collection (ATCC, Manassas, Va.), and cultivated in DMEM or RPMI 1640 medium (Gibco Invitrogen Corp., Carlsbad, Calif.), supplemented with 10% (v/v) heat-inactivated Fetal Bovine Serum (Gemini Bio-Products, Woodland, Calif.) and 1000 mcg/ml penicillin G sodium, 1000 mcg/ml streptomycin sulfate, and 2.5 mcg/ml amphotericin B (Antibiotic-Antimycotic, Gibco Invitrogen Corp.) as recommended by ATCC. When indicated, tumor cells were irradiated with 20,000 Rads of γ-radiation emitted by a Model C-188 Cobalt-60 source (MDS-Nordion, Ottawa, ON, Canada).

For animal treatment, mice were first sheared of fur on their right flank skin, then injected with either phosphate-buffered saline (PBS, Sigma, St. Louis, Mo.) alone, 100 mcg Clone 1 or Clone 2 anti-TIM-1 antibody, or 10⁶ γ-irradiated EL4, B16F10, or P815 cells plus either 100 mcg Clone 1 or Clone 2 antibody in PBS vehicle. These injections occurred 10, 17, and 32 days prior to injection of animals with the respective number of live tumor cells (see above), freshly prepared from cultures in logarithmic-growth phase. Tumor challenge injections were given into the sheared left flank skin. All challenge and pre-challenge injections were delivered by subcutaneous route in volumes of 100 μl, accomplished using 26-gauge, ⅝-inch subcutaneous bevel hypodermic needles (BD Medical Systems, Franklin Lakes, N.J.).

For tumor measurement and statistical analyses, tumors growing under the left flank skin of tumor-challenged mice were measured using digital calipers (Mitutoyo America Corp., Aurora, Ill.) 10, 13, 17, 23, and 26 days after subcutaneous delivery of tumor challenge cells. Tumor measurements in millimeters were collected on three roughly perpendicular axes, representing tumor length (L), width (W), and height from the surrounding body contour (H). Tumor volumes were calculated by applying the formula: Volume=[(4/3)·π·(L/2) (W/2)·(H/2)]. Standard Error of the Mean (SEM) and Student's t test probability (p) values were determined using Microsoft Excel software.

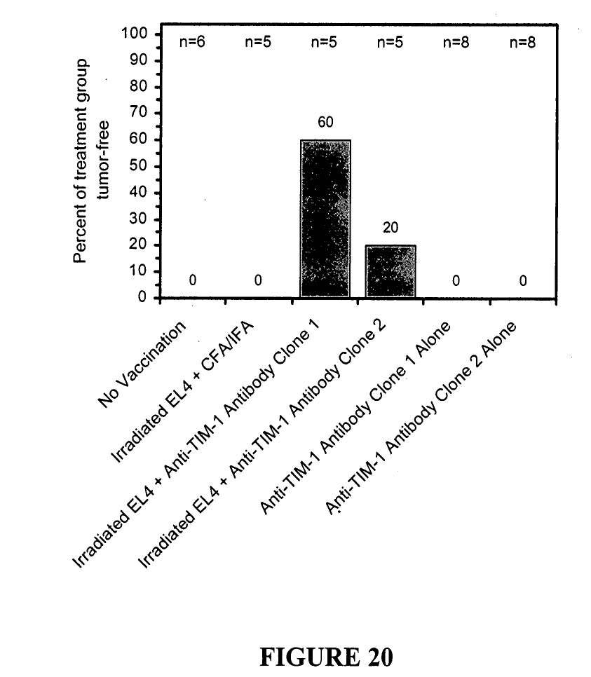

As shown in FIG. 20, delivering anti-TIM-1 antibodies with vaccination elicits complete tumor rejection. Mice were injected 10, 17, and 32 days prior to tumor challenge with the indicated materials. γ-irradiated (20,000 Rad) EL4 tumor cells were delivered at 10⁶ cells per injection. Anti-TIM-1 antibodies were delivered at 100 mcg per injection. All injections were accomplished by subcutaneous delivery of 100 μl volumes to the sheared right flank skin of C57BL/6 female mice. At day 0, mice were challenged with subcutaneous injection of 10⁶ live EL4 tumor cells to the sheared left flank skin, which was delivered in a volume of 100 μl PBS. Data shown are for day 26 post-challenge. These results show that delivering anti-TIM-1 antibodies with vaccination elicits complete tumor rejection.

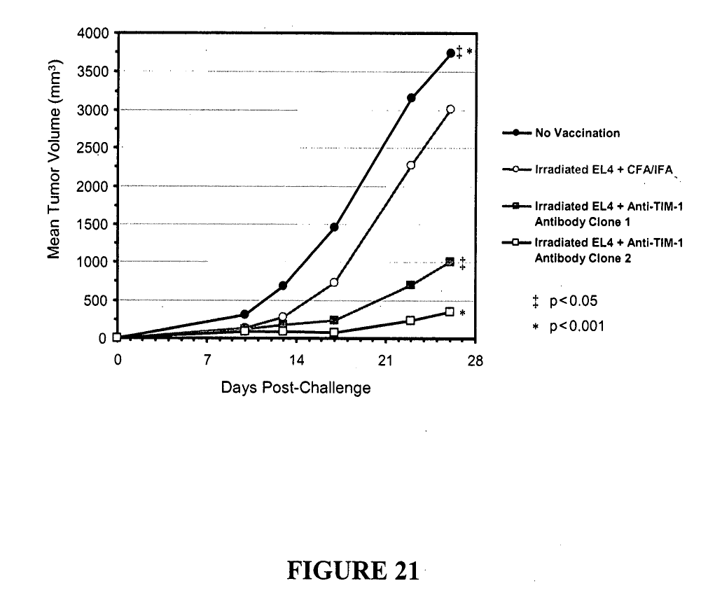

As shown in FIG. 21, vaccines supplemented with anti-TIM-1 antibodies greatly inhibit tumor growth upon challenge with live tumor cells. Mice were injected 10, 17, and 32 days prior to tumor challenge with the indicated materials. γ-irradiated (20,000 Rad) EL4 tumor cells were delivered at 10⁶ cells per injection. Anti-TIM-1 antibodies were delivered at 100 mcg per injection. All injections were accomplished by subcutaneous delivery of 100 μl volumes to the sheared right flank skin of C57BL/6 female mice. At day 0, mice were challenged with subcutaneous injection of 10⁶ live EL4 tumor cells to the sheared left flank skin, which was delivered in a volume of 100 μl PBS. Tumor volumes were measured over the following 26 days, and statistical significance was determined by applying unpaired, two-tailed Student's t test calculations. These results show that vaccines supplemented with anti-TIM-1 antibodies greatly inhibit tumor growth upon challenge with live tumor cells.

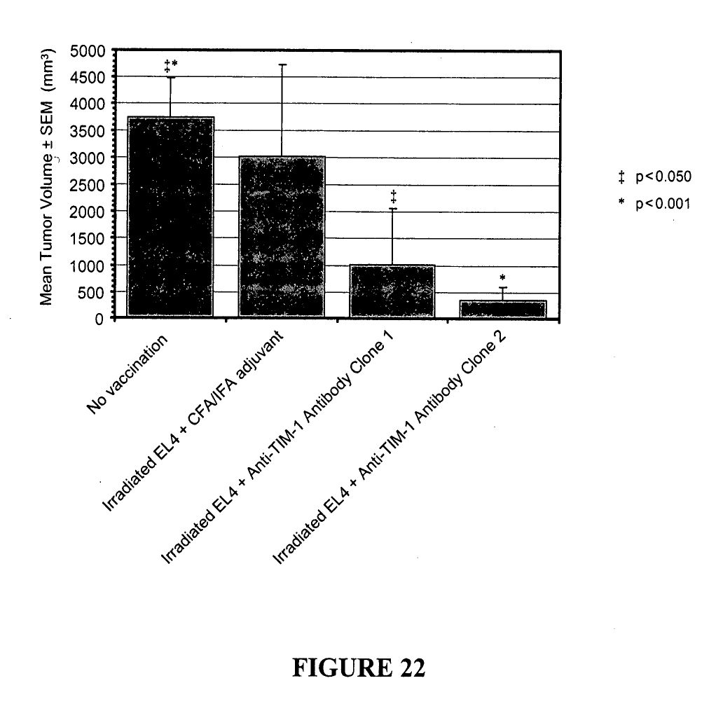

As shown in FIG. 22, vaccines supplemented with anti-TIM-1 antibodies greatly inhibit tumor growth upon challenge with live tumor cells. Mice were injected 10, 17, and 32 days prior to tumor challenge with the indicated materials. γ-irradiated (20,000 Rad) EL4 tumor cells were delivered at 10⁶ cells per injection. Anti-TIM-1 antibodies were delivered at 100 mcg per injection. All injections were accomplished by subcutaneous delivery of 100 μl volumes to the sheared right flank skin of C57BL/6 female mice. At day 0, mice were challenged with subcutaneous injection of 10⁶ live EL4 tumor cells to the sheared left flank skin, which was delivered in a volume of 100 μl PBS. Tumor volumes were measured after 26 days, and statistical significance was determined by applying unpaired, two-tailed Student's t test calculations. Data shown are for day 26 post-challenge. These results show that vaccines supplemented with anti-TIM-1 antibodies greatly inhibit tumor growth upon challenge with live tumor cells.

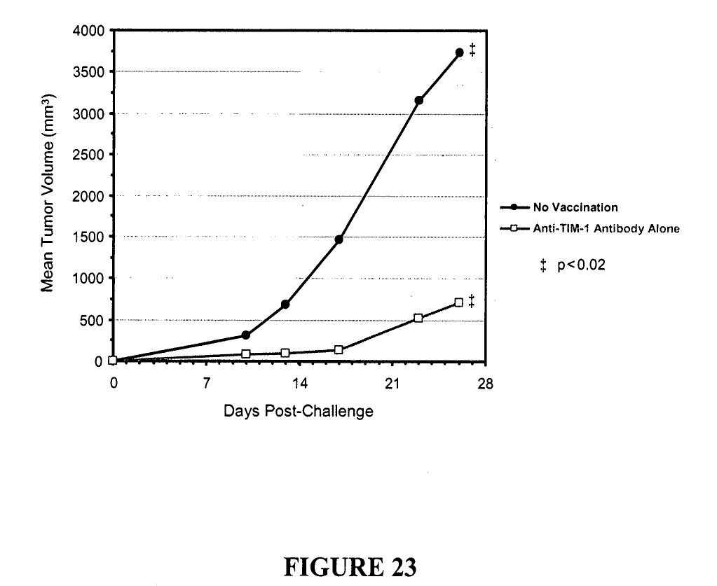

As shown in FIG. 23, pre-treatment of animals with anti-TIM-1 antibody prior to live tumor cell challenge significantly restrains tumor growth. Mice were injected 10, 17, and 32 days prior to tumor challenge with 100 mcg anti-TIM-1 antibody per injection. Injections were accomplished by subcutaneous delivery of 100 μl volumes to the sheared right flank skin of C57BL/6 female mice. At day 0, mice were challenged with subcutaneous injection of 10⁶ live EL4 tumor cells to the sheared left flank skin, which was delivered in a volume of 100 μl PBS. Tumor volumes were measured over the following 26 days, and statistical significance was determined by applying unpaired, two-tailed Student's t test calculations. These results show that pre-treatment of animals with anti-TIM-1 antibody prior to live tumor cell challenge significantly restrains tumor growth.

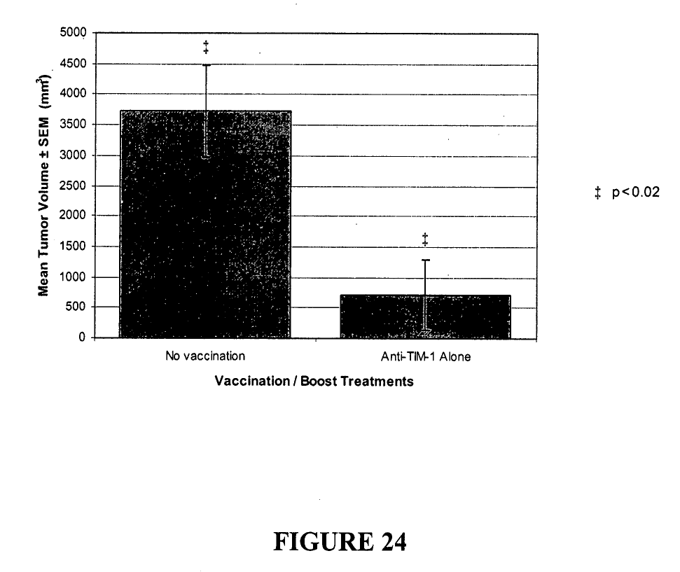

As shown in FIG. 24, pre-treatment of animals with anti-TIM-1 antibody prior to live tumor cell challenge significantly limits tumor growth. Mice were injected 10, 17, and 32 days prior to tumor challenge with 100 mcg anti-TIM-1 antibodies. γ-irradiated (20,000 Rad) EL4 tumor cells were delivered at 10⁶ cells per injection. Injections were accomplished by subcutaneous delivery of 100 μl volumes to the sheared right flank skin of C57BL/6 female mice. At day 0, mice were challenged with subcutaneous injection of 10⁶ live EL4 tumor cells to the sheared left flank skin, which was delivered in a volume of 100 μl PBS. Tumor volumes were measured after 26 days, and statistical significance was determined by applying unpaired, two-tailed Student's t test calculations. Data shown are for day 26 post-challenge. These results show that pre-treatment of animals with anti-TIM-1 antibody prior to live tumor cell challenge significantly limits tumor growth.

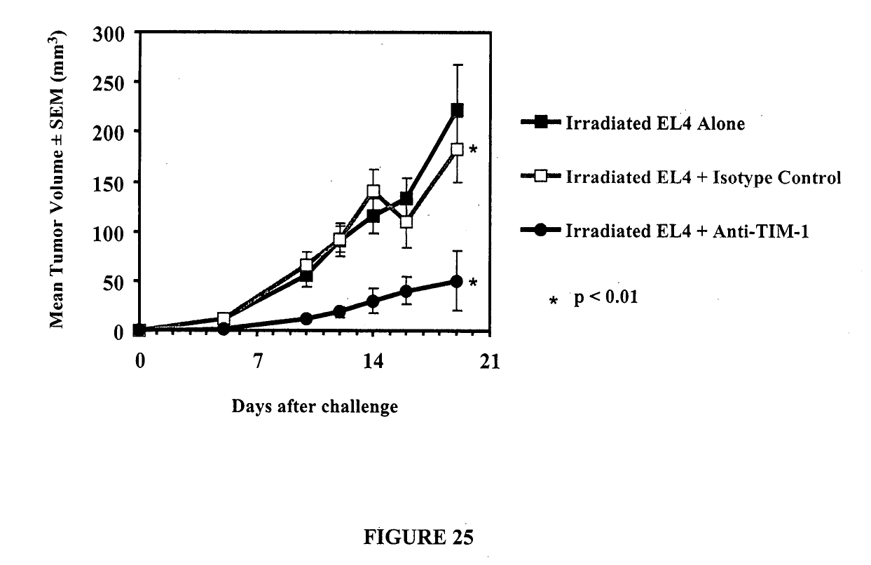

As shown in FIG. 25, anti-TIM-1 enhances tumor vaccine effectiveness. C57BL/6 mice received primary vaccination with 10⁶ gamma-irradiated (20,000 Rad) EL4 tumor cells, delivered by subcutaneous injection. At the same time, either 100 μl phosphate buffered saline (PBS) vehicle control, or 100 mcg anti-TIM-1 antibody or 100 mcg rIgG2b isotype control antibody in 100 μl PBS vehicle was delivered intraperitoneally. Three weeks after primary vaccination, mice received a first boost with identical preparations. This was followed two weeks later by a second, identical boost. Eleven days after this second boost, mice were challenged with a subcutaneous injection of 10⁶ live EL4 tumor cells, delivered contralaterally to the site of vaccination and boost dosing. In all cases, mice receiving live tumor cells developed measurable tumor masses by 10 days post-challenge. Tumor diameters were measured using digital calipers at several points during the 19 days following live tumor cell challenge. Diameters of three roughly perpendicular axes of each tumor, length (L), width (W), and height (H), were recorded at each time point. Tumor volumes were calculated using the formula volume (V)=(4/3)·π·(L/2)·(W/2)·(H/2). Treatment group mean tumor volumes were calculated using Microsoft Excel. P values were determined by Student's t test, calculated using Microsoft Excel. Anti-TIM-1 monoclonal antibody was purchased from R&D Systems Inc. (Minneapolis Minn.)(mAb AF1817). These results show that anti-TIM-1 enhances tumor vaccine effectiveness.

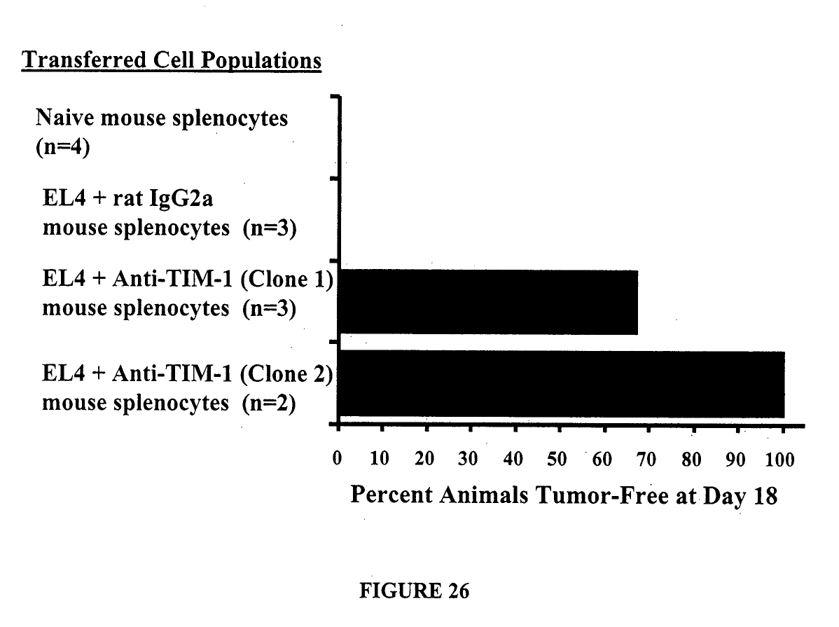

As shown in FIG. 26, vaccination with anti-TIM-1 adjuvants drives generation of protective immunity. Naive C57BL/6 mice were vaccinated with an admixture of 10⁶ gamma-irradiated (20,000 Rad) EL4 tumor cells, either alone in 100 μl phosphate buffered saline (PBS), or with 100 mcg anti-TIM-1 antibody or 100 mcg rIgG2a isotype control antibody in 100 μl PBS, delivered by subcutaneous injection. This was followed fifteen days later by boosting using an identical method. A second boost by the same method followed seven days after the first. Ten days after this second boost, mice were challenged with a subcutaneous injection of 10⁶ live EL4 tumor cells, delivered contralaterally to the site of vaccination and boost dosing. Splenocytes were recovered from mice rejecting the EL4 tumor challenge 31 days after challenge with live tumor cells. Similarly, splenocytes were also recovered from rIgG2a control group mice, and age-matched naive C57BL/6 mice. After red blood cell depletion in vitro, 10⁷ splenocytes from either the anti-TIM-1, rIgG2a, or naive mice were adoptively transferred into naive C57BL/6 recipient animals by tail vein injection. One day after transfer, recipient mice were challenged with subcutaneous injection of 10 live EL4 tumor cells. Eighteen days after adoptive transfer, mice were evaluated for the presence of palpable tumor masses under the skin at the site of prior subcutaneous live tumor challenge. Animals presenting no detectable tumor mass were deemed to be tumor free and are indicated as a percentage of the total animals receiving the identical adoptive transfer treatment. These results show that adoptive transfer induces tumor rejection.

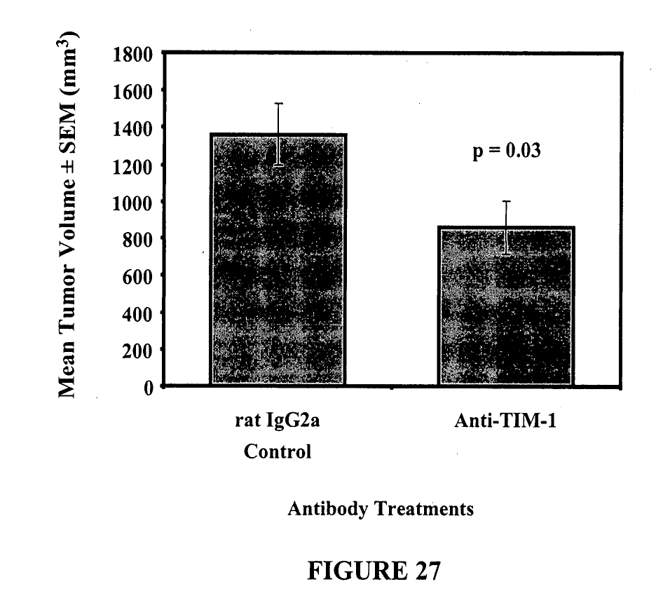

As shown in FIG. 27, anti-TIM-1 therapy slows tumor growth. Naive C57BL/6 mice were challenged by subcutaneous injection of 10⁶ live EL4 tumor cells, then treated six days later with one intraperitoneal injection of 100 mcg anti-TIM-1 antibody, or 100 mcg rIgG2a control antibody. Individual animal tumors were measured fifteen days after delivery of the anti-TIM-1 or control antibody treatments. Tumor diameters were recorded for three roughly perpendicular axes of each tumor, length (L), width (W), and height (H). Tumor volumes were calculated using the formula volume (V)=(4/3)·π·(L/2)·(W/2)·(H/2). Group mean tumor volumes and the standard error for each calculated mean (SEM) were calculated using Microsoft Excel software. P values were determined by Student's t test, calculated using Microsoft Excel. These results show that anti-TIM-1 therapy slows tumor growth.

Both, anti-TIM-1 and TIM-4/Fc have been demonstrated to enhance Th1 immunity (see Example XIV). Therefore, TIM-4/Fc acts both as a tumor vaccine adjuvant and as a therapeutic agent for the treatment of tumors, as shown in the experimental studies shown in Example XII.

### EXAMPLE XII

### TIM-3/Fc and Anti-TIM-3 as Adjuvants for Cancer Vaccines and as Therapeutic Agents for the Treatment of Tumors

This example describes adjuvant activity of TIM-3/Fc and anti-TIM-3 for cancer vaccines and therapeutic treatment of tumors.

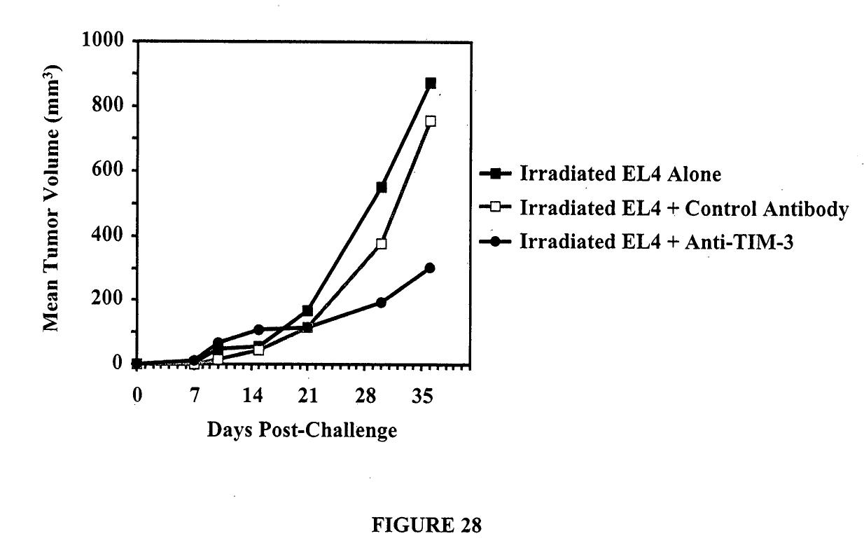

As shown in FIG. 28, TIM-3-specific antibody reduces tumor growth when used as a vaccine adjuvant. Naive C57BL/6 mice received primary vaccination with an admixture of 10⁶ gamma-irradiated (20,000 Rad) EL4 tumor cells, either alone in 100 μl phosphate buffered saline (PBS) vehicle, or with 100 mcg anti-TIM-3 antibody, or 100 mcg rIgG2a isotype control antibody in PBS. Two weeks after primary vaccination, mice received a boost injection identical to primary vaccination. Ten days after this boost, mice were challenged by a subcutaneous injection of 10⁶ live EL4 tumor cells, delivered contralaterally to the site of vaccination and boost dosing. In all cases, mice receiving live tumor cell developed measurable tumor masses by day 10 post-challenge. During the 36 days following tumor challenge, tumor diameters were measured using digital calipers. Tumor diameters were recorded for three roughly perpendicular axes of each tumor, length (L), width (W), and height (H), at several time points. Tumor volumes were calculated using the formula volume (V)=(4/3)·π·(L/2)·(W/2)·(H/2). Treatment group mean tumor volumes were calculated using Microsoft Excel. These results show that tumor vaccination in the presence of anti-TIM-3 restrains tumor growth.

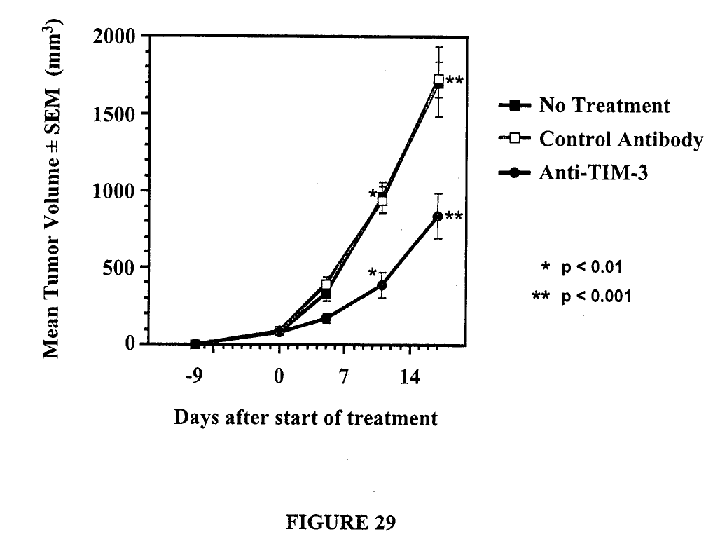

As shown in FIG. 29, anti-TIM-3 therapy limits tumor growth. Naive C57BL/6 mice were challenged with subcutaneous injection of 10⁶ live EL4 tumor cells, then treated nine days later with one intraperitoneal injection of 100 mcg anti-TIM-3 antibody, or 100 mcg rIgG2a isotype control antibody. Individual animal tumors were measured at the time of therapy using digital calipers, and at several time points after treatment with anti-TIM-3 or control antibody. Tumor diameters were recorded for three roughly perpendicular axes of each tumor, length (L), width (W), and height (H). Tumor volumes were calculated using the formula volume (V)=(4/3)·π·(L/2)·(W/2)·(H/2). Treatment group means and the standard error for each calculated mean (SEM) were calculated using Microsoft Excel. P values were determined by two-way ANOVA statistical analysis, calculated using GraphPad Prism software (GraphPad Software; San Diego Calif.). These results show that anti-TIM-3 therapy limits tumor growth.

Both anti-TIM-3 and TIM-3/Fc have been demonstrated to enhance Th1 immunity and to exacerbate disease in Th1 disease models (Monney et al., Nature 415:536-541 (2002); Sabatos et al., Nature Immunol. 4:1102-1110 (2003)). Therefore, TIM-3/Fc acts both as a tumor vaccine adjuvant and as a therapeutic agent for the treatment of tumors, as demonstrated in the experimental studies shown in FIGS. 28 and 29.

### EXAMPLE XIV

### Both Anti-TIM-1 and TIM-4/Fc Stimulate Immune Responses of a Th1 Driven Immune Reaction in Mice

Six to eight week old female SJL/J mice (Jackson Laboratories) were immunized with 100 mcg of PLP139-151 peptide emulsified in complete Freund's adjuvant (CFA) in the right and left flanks to stimulate a Th1 immune response against the peptide. Following the injection of PLP 139-151 in CFA, 100 ng of pertussis toxin was injected i.v. (tail vein). A second dose of 100 ng of pertussis toxin was administered 48 hours later. IgG2a isotype control antibody (100 mcg/mouse), TIM-1 monoclonal antibody (100 mcg) or TIM-4/Fc were administered intraperitoneally (i.p.) subsequent to immunization with PLP. The animals were monitored for the development of immunological responses to the antigen. The results indicate that both TIM-1 antibodies and TIM-4/Fc stimulate immune responses against the PLP peptide, as monitored by measuring T cell proliferation in response to re-exposure to the PLP peptide, and by IL-4 and IFN-gamma cytokine ELISAs.

These results show that TIM targeting molecules, exemplified as anti-TIM-1 antibodies, can be used to inhibit tumor growth.

### EXAMPLE XV

### Mouse and Human Tumor Cell Lines Expressing TIM-1 and TIM-3 as Well as TIM Ligands

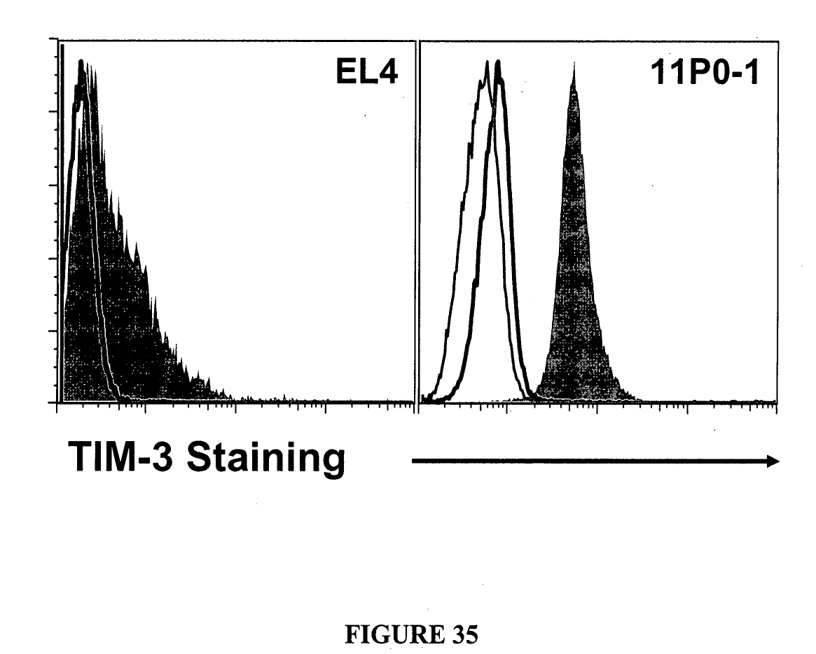

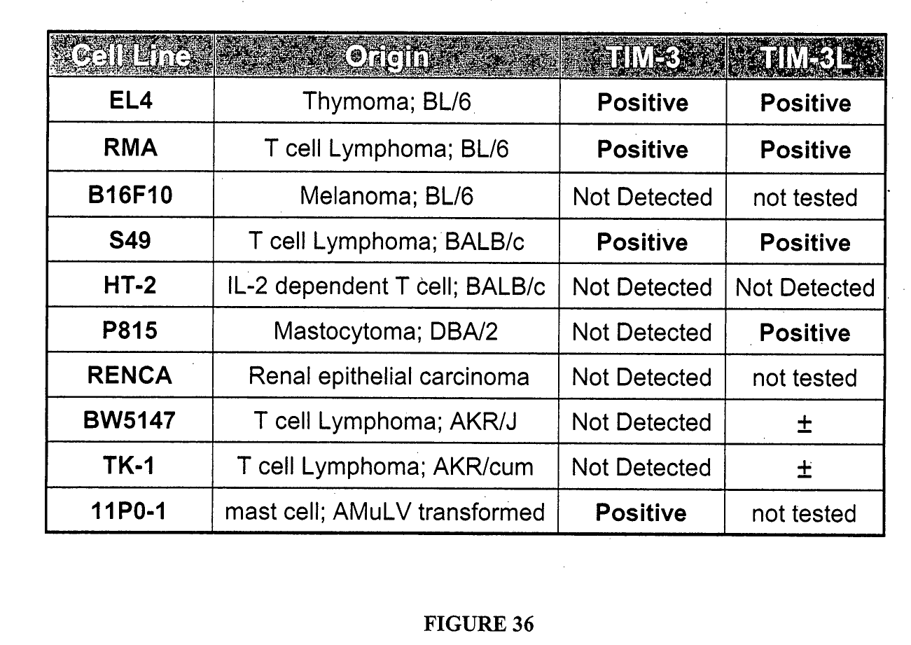

Mouse and human tumor cell lines were analyzed for TIM-1 and TIM-3 expression by fluorescence activated cell sorting (FACS) analysis. Cultured tumor cell lines were incubated in the presence of either control, TIM-1 or TIM-3 monoclonal antibodies, and the binding of the TIM-specific antibodies was detected by either direct conjugation of the TIM antibodies using a fluorescent tag or by use of fluorescently labeled secondary antibodies. TIM-1 expression was detected on the human renal adenocarcinoma cell line 769-P (FIG. 33) as well as on the human hepatocellular carcinoma HepG2. TIM-1 expression was also detected on the mouse renal adenocarcinoma RAG. TIM-3 expression was detected on several different tumors, including thymomas and lymphomas, as shown in FIG. 35 and summarized in FIG. 36. Using TIM-3/Fc, the expression of TIM-3 ligand on tumor cell lines was also analyzed. As summarized in FIG. 36, various tumors expressing TIM-3 ligand (TIM-3L) were identified, including thymomas, lymphomas and mastocytomas.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

1. A composition comprising an antigen and a TIM targeting molecule in a pharmaceutically acceptable carrier.

2. The composition of claim 1, wherein said TIM targeting molecule is a TIM antibody.

3. The composition of claim 2, wherein said TIM antibody is specific for a TIM selected from TIM-1, TIM-2, TIM-3 and TIM-4.

4. The composition of claim 1, wherein said TIM targeting molecule is a TIM-Fc fusion polypeptide.

5. The composition of claim 4, wherein the Fc portion of said TIM-Fc fusion polypeptide is target-cell depleting.

6. The composition of claim 4, wherein the Fc portion of said TIM-Fc fusion polypeptide is non target-cell depleting.

7. The composition of claim 4, wherein the TIM portion of said TIM-Fc fusion polypeptide is selected from TIM-1, TIM-2, TIM-3 and TIM-4.

8. The composition of claim 1, wherein said antigen is selected from a viral, bacterial, parasitic, and tumor associated antigen.

9. A composition comprising a TIM targeting molecule conjugated to a therapeutic or diagnostic moiety.

10. The composition of claim 9, wherein the therapeutic moiety is selected from a chemotherapeutic agent, cytotoxic agent and toxin.

11. The composition of claim 10, wherein the cytotoxic agent is a radionuclide or chemical compound.

12. The composition of claim 11, wherein the chemical compound is selected from calicheamicin, esperamicin, duocarmycin, doxorubicin, melphalan, methotrexate, chlorambucil, cytarabine, vindesine, cis-platinum, etoposide, bleomycin, mitomycin C and 5-fluorouracil.

13. The composition of claim 11, wherein the radionuclide is Iodine-131 or Yttrium-90.

14. The composition of claim 10, wherein the toxin is a plant or bacterial toxin.

15. The composition of claim 14, wherein the plant toxin is selected from ricin, abrin, pokeweed antiviral protein, saporin and gelonin.

16. The composition of claim 14, wherein the bacterial toxin is selected from Pseudomonas exotoxin, and diphtheria toxin.

17. The composition of claim 9, wherein said TIM targeting molecule is a TIM antibody.

18. The composition of claim 17, wherein said TIM antibody is specific for a TIM selected from TIM-1, TIM-2, TIM-3 and TIM-4.

19. The composition of claim 9, wherein said TIM targeting molecule is a TIM-Fc fusion polypeptide.

20. The composition of claim 19, wherein the Fc portion of said TIM-Fc fusion polypeptide is target-cell depleting.

21. The composition of claim 19, wherein the Fc portion of said TIM-Fc fusion polypeptide is non target-cell depleting.

22. The composition of claim 19, wherein the TIM portion of said TIM-Fc fusion polypeptide is selected from TIM-1, TIM-2, TIM-3 and TIM-4.

23. A method of stimulating an immune response in an individual, comprising administering a composition comprising an antigen and a TIM targeting molecule in a pharmaceutically acceptable carrier.

24. The method of claim 23, wherein said TIM targeting molecule is a TIM antibody.

25. The method of claim 24, wherein said TIM antibody is specific for a TIM selected from TIM-1, TIM-2, TIM-3 and TIM-4.

26. The method of claim 23, wherein said TIM targeting molecule is a TIM-Fc fusion polypeptide.

27. The method of claim 26, wherein the TIM portion of said TIM-Fc fusion polypeptide is selected from TIM-1, TIM-2, TIM-3 and TIM-4.

28. The method of claim 23, wherein said antigen is selected from a viral, bacterial, parasitic, and tumor associated antigen.

29. The method of claim 28, wherein said antigen is a peptide.

30. A method of prophylactic treatment of a disease, comprising administering to an individual a composition comprising an antigen and a TIM targeting molecule in a pharmaceutically acceptable carrier.

31. The method of claim 30, wherein said TIM targeting molecule is a TIM antibody.

32. The method of claim 31, wherein said TIM antibody is specific for a TIM selected from TIM-1, TIM-2, TIM-3 and TIM-4.

33. The method of claim 30, wherein said TIM targeting molecule is a TIM-Fc fusion polypeptide.

34. The method of claim 33, wherein the TIM portion of said TIM-Fc fusion polypeptide is selected from TIM-1, TIM-2, TIM-3 and TIM-4.

35. The method of claim 30, wherein the disease is an infectious disease.

36. The method of claim 35, wherein said antigen is selected from a viral, bacterial, and parasitic antigen.

37. The method of claim 30, wherein the disease is cancer.

38. The method of claim 37, wherein said antigen is a tumor associated antigen.

39. A method of ameliorating a sign or symptom associated with a disease, comprising administering to an individual a composition comprising an antigen and a TIM targeting molecule in a pharmaceutically acceptable carrier.

40. The method of claim 39, wherein said TIM targeting molecule is a TIM antibody.

41. The method of claim 39, wherein said TIM antibody is specific for a TIM selected from TIM-1, TIM-2, TIM-3 and TIM-4.

42. The method of claim 39, wherein said TIM targeting molecule is a TIM-Fc fusion polypeptide.

43. The method of claim 42, wherein the TIM portion of said TIM-Fc fusion polypeptide is selected from TIM-1, TIM-2, TIM-3 and TIM-4.

44. The method of claim 39, wherein the disease is an infectious disease.

45. The method of claim 44, wherein said antigen is selected from a viral, bacterial, and parasitic antigen.

46. The method of claim 39, wherein the disease is cancer.

47. The method of claim 46, wherein said antigen is a tumor associated antigen.

48. A method of targeting a tumor, comprising administering a TIM targeting molecule to a subject, wherein said tumor expresses a TIM or TIM ligand.

49. The method of claim 48, wherein said TIM targeting molecule is administered with an antigen.

50. The method of claim 49, wherein said antigen is a tumor associated antigen.

51. The method of claim 48, wherein said TIM targeting molecule is a TIM antibody.

52. The method of claim 51, wherein said TIM antibody is specific for a TIM selected from TIM-1, TIM-2, TIM-3 and TIM-4.

53. The method of claim 48, wherein said TIM targeting molecule is a TIM-Fc fusion polypeptide.

54. The method of claim 53, wherein the Fc portion of said TIM-Fc fusion polypeptide is target-cell depleting.

55. The method of claim 53, wherein the Fc portion of said TIM-Fc fusion polypeptide is non target-cell depleting.

56. The method of claim 53, wherein the TIM portion of said TIM-Fc fusion polypeptide is selected from TIM-1, TIM-2, TIM-3 and TIM-4.

57. The method of claim 48, wherein the tumor is selected from a carcinoma, sarcoma and lymphoma.

58. The method of claim 48, wherein said TIM targeting molecule is conjugated to a therapeutic moiety.

59. The method of claim 58, wherein the therapeutic moiety is selected from a chemotherapeutic agent, cytotoxic agent and toxin.

60. The method of claim 59, wherein the cytotoxic agent is a radionuclide or chemical compound.

61. The method of claim 60, wherein the chemical compound is selected from calicheamicin, esperamicin, duocarmycin, doxorubicin, melphalan, methotrexate, chlorambucil, cytarabine, vindesine, cis-platinum, etoposide, bleomycin, mitomycin C and 5-fluorouracil.

62. The method of claim 60, wherein the radionuclide is Iodine-131 or Yttrium-90.

63. The method of claim 59, wherein the toxin is a plant or bacterial toxin.

64. The method of claim 63, wherein the plant toxin is selected from ricin, abrin, pokeweed antiviral protein, saporin and gelonin.

65. The method of claim 63, wherein the bacterial toxin is selected from Pseudomonas exotoxin, and diphtheria toxin.

66. The method of claim 58, wherein said TIM targeting molecule is a TIM antibody.

67. The method of claim 66, wherein said TIM antibody is specific for a TIM selected from TIM-1, TIM-2, TIM-3 and TIM-4.

68. The method of claim 58, wherein said TIM targeting molecule is a TIM-Fc fusion polypeptide.

69. The method of claim 68, wherein the Fc portion of said TIM-Fc fusion polypeptide is target-cell depleting.

70. The method of claim 68, wherein the Fc portion of said TIM-Fc fusion polypeptide is non target-cell depleting.

71. The method of claim 68, wherein the TIM portion of said TIM-Fc fusion polypeptide is selected from TIM-1, TIM-2, TIM-3 and TIM-4.

72. A method of inhibiting tumor growth, comprising administering a TIM targeting molecule to a subject, wherein said tumor expresses a TIM or TIM ligand.

73. The method of claim 72, wherein said TIM targeting molecule is administered with an antigen.

74. The method of claim 72, wherein said TIM targeting molecule is a TIM antibody.

75. The method of claim 74, wherein said TIM antibody is specific for a TIM selected from TIM-1, TIM-2, TIM-3 and TIM-4.

76. The method of claim 72, wherein said TIM targeting molecule is a TIM-Fc fusion polypeptide.

77. The method of claim 76, wherein the Fc portion of said TIM-Fc fusion polypeptide is target-cell depleting.

78. The method of claim 76, wherein the Fc portion of said TIM-Fc fusion polypeptide is non target-cell depleting.

79. The method of claim 76, wherein the TIM portion of said TIM-Fc fusion polypeptide is selected from TIM-1, TIM-2, TIM-3 and TIM-4.

80. The method of claim 72, wherein the tumor is selected from a carcinoma, sarcoma and lymphoma.

81. The method of claim 72, wherein said TIM targeting molecule is conjugated to a therapeutic moiety.

82. The method of claim 81, wherein the therapeutic moiety is selected from a chemotherapeutic agent, cytotoxic agent and toxin.

83. The method of claim 82, wherein the cytotoxic agent is a radionuclide or chemical compound.

84. The method of claim 83, wherein the chemical compound is selected from calicheamicin, esperamicin, duocarmycin, doxorubicin, melphalan, methotrexate, chlorambucil, cytarabine, vindesine, cis-platinum, etoposide, bleomycin, mitomycin C and 5-fluorouracil.

85. The method of claim 83, wherein the radionuclide is Iodine-131 or Yttrium-90.

86. The method of claim 82, wherein the toxin is a plant or bacterial toxin.

87. The method of claim 86, wherein the plant toxin is selected from ricin, abrin, pokeweed antiviral protein, saporin and gelonin.

88. The method of claim 86, wherein the bacterial toxin is selected from Pseudomonas exotoxin, and diphtheria toxin.

89. The method of claim 81, wherein said TIM targeting molecule is a TIM antibody.

90. The method of claim 89, wherein said TIM antibody is specific for a TIM selected from TIM-1, TIM-2, TIM-3 and TIM-4.

91. The method of claim 81, wherein said TIM targeting molecule is a TIM-Fc fusion polypeptide.

92. The method of claim 91, wherein the Fc portion of said TIM-Fc fusion polypeptide is target-cell depleting.

93. The method of claim 91, wherein the Fc portion of said TIM-Fc fusion polypeptide is non target-cell depleting.

94. The method of claim 91, wherein the TIM portion of said TIM-Fc fusion polypeptide is selected from TIM-1, TIM-2, TIM-3 and TIM-4.

95. A method of detecting a tumor, comprising administering a TIM targeting molecule conjugated to a diagnostic moiety to a subject, wherein said tumor expresses a TIM or TIM ligand.

96. The method of claim 95, wherein said TIM targeting molecule is a TIM antibody.

97. The method of claim 96, wherein said TIM antibody is specific for a TIM selected from TIM-1, TIM-2, TIM-3 and TIM-4.

98. The method of claim 95, wherein said TIM targeting molecule is a TIM-Fc fusion polypeptide.

99. The method of claim 98, wherein the TIM portion of said TIM-Fc fusion polypeptide is selected from TIM-1, TIM-2, TIM-3 and TIM-4.

100. A method of ameliorating a sign or symptom associated with an autoimmune disease, comprising administering a TIM targeting molecule to a subject.

101. The method of claim 100, wherein said autoimmune disease is selected from rheumatoid arthritis, multiple sclerosis, autoimmune diabetes mellitus, systemic lupus erythematosus, and autoimmune lymphoproliferative syndrome (ALPS).

102. The method of claim 100, wherein said TIM targeting molecule is administered with an antigen.

103. The method of claim 100, wherein said TIM targeting molecule is a TIM antibody.

104. The method of claim 103, wherein said TIM antibody is specific for a TIM selected from TIM-1, TIM-2, TIM-3 and TIM-4.

105. The method of claim 100, wherein said TIM targeting molecule is a TIM-Fc fusion polypeptide.

106. The method of claim 105, wherein the Fc portion of said TIM-Fc fusion polypeptide is target-cell depleting.

107. The method of claim 105, wherein the Fc portion of said TIM-Fc fusion polypeptide is non target-cell depleting.

108. The method of claim 105, wherein the TIM portion of said TIM-Fc fusion polypeptide is selected from TIM-1, TIM-2, TIM-3 and TIM-4.

109. The method of claim 100, wherein said TIM targeting molecule is conjugated to a therapeutic moiety.

110. The method of claim 109, wherein the therapeutic moiety is selected from a chemotherapeutic agent, cytotoxic agent and toxin.

111. The method of claim 110, wherein the cytotoxic agent is a radionuclide or chemical compound.

112. The method of claim 111, wherein the chemical compound is selected from calicheamicin, esperamicin, duocarmycin, doxorubicin, melphalan, methotrexate, chlorambucil, cytarabine, vindesine, cis-platinum, etoposide, bleomycin, mitomycin C and 5-fluorouracil.

113. The method of claim 111, wherein the radionuclide is Iodine-131 or Yttrium-90.

114. The method of claim 110, wherein the toxin is a plant or bacterial toxin.

115. The method of claim 114, wherein the plant toxin is selected from ricin, abrin, pokeweed antiviral protein, saporin and gelonin.

116. The method of claim 14, wherein the bacterial toxin is selected from Pseudomonas exotoxin, and diphtheria toxin.

117. The method of claim 109, wherein said TIM targeting molecule is a TIM antibody.

118. The method of claim 117, wherein said TIM antibody is specific for a TIM selected from TIM-1, TIM-2, TIM-3 and TIM-4.

119. The method of claim 109, wherein said TIM targeting molecule is a TIM-Fc fusion polypeptide.

120. The method of claim 119, wherein the Fc portion of said TIM-Fc fusion polypeptide is target-cell depleting.

121. The method of claim 119, wherein the Fc portion of said TIM-Fc fusion polypeptide is non target-cell depleting.

122. The method of claim 119, wherein the TIM portion of said TIM-Fc fusion polypeptide is selected from TIM-1, TIM-2, TIM-3 and TIM-4.
